SEQUENCE LISTING (223 sequences)

SEQ ID NO: 1 - DNA, 20 bp, synthetic construct
  source 1..20: mol_type = other DNA
gttgtccgctgtcacgactc

SEQ ID NO: 2 - DNA, 4356 bp, synthetic construct
  source 1..4356: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgtgcaggtgagtgatccaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacggatcaagaacctgctgacgttttagagctagaaatagcaagttaaaataaggctagtccgttatcaacttgaaaaagtgggaccgagtcggtcctttttttgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 3 - DNA, 10 bp, synthetic construct
  source 1..10: mol_type = other DNA
gcggctggag

SEQ ID NO: 4 - DNA, 42 bp, synthetic construct
  source 1..42: mol_type = other DNA
ctacacgacgctcttccgatctcgaggcagagggtccaaagc

SEQ ID NO: 5 - DNA, 43 bp, synthetic construct
  source 1..43: mol_type = other DNA
agacgtgtgctcttccgatctctttcaacccgaacggagacac

SEQ ID NO: 6 - DNA, 44 bp, synthetic construct
  source 1..44: mol_type = other DNA
ctacacgacgctcttccgatctgggctcactgctctccagagtg

SEQ ID NO: 7 - DNA, 46 bp, synthetic construct
  source 1..46: mol_type = other DNA
agacgtgtgctcttccgatctgatgattctcctacttcctcctcgg

SEQ ID NO: 8 - DNA, 51 bp, synthetic construct
  source 1..51: mol_type = other DNA
ctacacgacgctcttccgatctagtctcacatttttgtcccagaacttatt

SEQ ID NO: 9 - DNA, 47 bp, synthetic construct
  source 1..47: mol_type = other DNA
agacgtgtgctcttccgatctttttgtatttttagtagggacggggt

SEQ ID NO: 10 - DNA, 46 bp, synthetic construct
  source 1..46: mol_type = other DNA
ctacacgacgctcttccgatctgaaaagagaaaagccaacgggttc

SEQ ID NO: 11 - DNA, 45 bp, synthetic construct
  source 1..45: mol_type = other DNA
agacgtgtgctcttccgatctctgaaggagcacttggaagatggt

SEQ ID NO: 12 - DNA, 46 bp, synthetic construct
  source 1..46: mol_type = other DNA
ctacacgacgctcttccgatctgaaagcagaggctcagaggaaacc

SEQ ID NO: 13 - DNA, 47 bp, synthetic construct
  source 1..47: mol_type = other DNA
agacgtgtgctcttccgatctgcagagcaaactcaaaccagaaaatg

SEQ ID NO: 14 - DNA, 45 bp, synthetic construct
  source 1..45: mol_type = other DNA
ctacacgacgctcttccgatctcgccattttcaaactgctctagc

SEQ ID NO: 15 - DNA, 41 bp, synthetic construct
  source 1..41: mol_type = other DNA
agacgtgtgctcttccgatcttgttgacgggaagggggtgt

SEQ ID NO: 16 - DNA, 45 bp, synthetic construct
  source 1..45: mol_type = other DNA
ctacacgacgctcttccgatctgagattatatcccgcacatggct

SEQ ID NO: 17 - DNA, 45 bp, synthetic construct
  source 1..45: mol_type = other DNA
agacgtgtgctcttccgatctggtggagtctacagaggcagtcag

SEQ ID NO: 18 - DNA, 44 bp, synthetic construct
  source 1..44: mol_type = other DNA
ctacacgacgctcttccgatcttccacgggagatggcttatgtc

SEQ ID NO: 19 - DNA, 48 bp, synthetic construct
  source 1..48: mol_type = other DNA
agacgtgtgctcttccgatctctgggttcttaaacactaggctactcc

SEQ ID NO: 20 - DNA, 44 bp, synthetic construct
  source 1..44: mol_type = other DNA
ctacacgacgctcttccgatctacttgaacctcacgctgtgggc

SEQ ID NO: 21 - DNA, 39 bp, synthetic construct
  source 1..39: mol_type = other DNA
agacgtgtgctcttccgatcttgctccccaagggcgtcc

SEQ ID NO: 22 - DNA, 20 bp, synthetic construct
  source 1..20: mol_type = other DNA
ggcggcttctccctcagtca

SEQ ID NO: 23 - DNA, 22 bp, synthetic construct
  source 1..22: mol_type = other DNA
ctttcaacccgaacggagacac

SEQ ID NO: 24 - DNA, 43 bp, synthetic construct
  source 1..43: mol_type = other DNA
tcgacggtatcgataagcttgatggcggcttctccctcagtca

SEQ ID NO: 25 - DNA, 45 bp, synthetic construct
  source 1..45: mol_type = other DNA
tcgacggtatcgataagcttgatcccaccctctaccctccctgat

SEQ ID NO: 26 - DNA, 20 bp, synthetic construct
  source 1..20: mol_type = other DNA
caggacaggcagccacaagg

SEQ ID NO: 27 - DNA, 25 bp, synthetic construct
  source 1..25: mol_type = other DNA
gatgattctcctacttcctcctcgg

SEQ ID NO: 28 - DNA, 29 bp, synthetic construct
  source 1..29: mol_type = other DNA
aatgatacggcgaccaccgagatctacac

SEQ ID NO: 29 - DNA, 54 bp, synthetic construct
  source 1..54: mol_type = other DNA
gtgactggagttcagacgtgtgctcttccgatctgttgtccgctgtcacgactc

SEQ ID NO: 30 - DNA, 54 bp, synthetic construct
  source 1..54: mol_type = other DNA
gtgactggagttcagacgtgtgctcttccgatctgagtcgtgacagcggacaac

SEQ ID NO: 31 - DNA, 44 bp, synthetic construct
  source 1..44: mol_type = other DNA
ctacacgacgctcttccgatctgattatgggagaactggagcct

SEQ ID NO: 32 - DNA, 49 bp, synthetic construct
  source 1..49: mol_type = other DNA
agacgtgtgctcttccgatcttatatgtagactaaccgattgaatatgg

SEQ ID NO: 33 - DNA, 46 bp, synthetic construct
  source 1..46: mol_type = other DNA
ctacacgacgctcttccgatctggctccgtaactttctatccgtcc

SEQ ID NO: 34 - DNA, 45 bp, synthetic construct
  source 1..45: mol_type = other DNA
agacgtgtgctcttccgatctgagacaagcagacagacgggtgag

SEQ ID NO: 35 - DNA, 22 bp, synthetic construct
  source 1..22: mol_type = other DNA
accaccatccgctctgccctat

SEQ ID NO: 36 - DNA, 19 bp, synthetic construct
  source 1..19: mol_type = other DNA
cgtggtgccgctccctttg

SEQ ID NO: 37 - DNA, 44 bp, synthetic construct; identical to SEQ ID NO: 31
  source 1..44: mol_type = other DNA

SEQ ID NO: 38 - DNA, 49 bp, synthetic construct; identical to SEQ ID NO: 32
  source 1..49: mol_type = other DNA

SEQ ID NO: 39 - DNA, 24 bp, synthetic construct
  source 1..24: mol_type = other DNA
tgacattgctatggagagcctcta

SEQ ID NO: 40 - DNA, 45 bp, synthetic construct
  source 1..45: mol_type = other DNA
ctacacgacgctcttccgatctgctctaaagtggagttgattgcg

SEQ ID NO: 41 - DNA, 39 bp, synthetic construct
  source 1..39: mol_type = other DNA
agacgtgtgctcttccgatctccccctggcgaagtttga

SEQ ID NO: 42 - DNA, 60 bp, synthetic construct
  source 1..60: mol_type = other DNA
gtggaaaggacgaaacaccgccctaatgattttgacttcagttttagagctagaaatagc

SEQ ID NO: 43 - DNA, 81 bp, synthetic construct
  source 1..81: mol_type = other DNA
cgataagcttgatatcgaattcaaaaaataatgattttgacttcaccgtaactgggaggaccgactcggtcccactttttc

SEQ ID NO: 44 - DNA, 98 bp, synthetic construct
  source 1..98: mol_type = other DNA
gataagcttgatatcgaattcaaaaaataatgattttgactgttgtccgctgtcacgactctcatggtaactgggaggaccgactcggtcccactttt

SEQ ID NO: 45 - DNA, 61 bp, synthetic construct
  source 1..61: mol_type = other DNA
tgtggaaaggacgaaacaccgaccattaaagaaaatatcatgttttagagctagaaatagc

SEQ ID NO: 46 - DNA, 76 bp, synthetic construct
  source 1..76: mol_type = other DNA
cgataagcttgatatcgaattcaaaaaaattaaagaaaatatcatctttggtgtttggaccgactcggtcccactt

SEQ ID NO: 47 - DNA, 89 bp, synthetic construct
  source 1..89: mol_type = other DNA
cgataagcttgatatcgaattcaaaaaaattaaagaaaatatgttgtccgctgtcacgactccatcggtggaccgactcggtcccactt

SEQ ID NO: 48 - DNA, 61 bp, synthetic construct
  source 1..61: mol_type = other DNA
tgtggaaaggacgaaacaccgatccttccagtcagggccatgttttagagctagaaatagc

SEQ ID NO: 49 - DNA, 80 bp, synthetic construct
  source 1..80: mol_type = other DNA
cgataagcttgatatcgaattcaaaaaaacagtcagggccataggatatacggttcaggtggaccgactcggtcccactt

SEQ ID NO: 50 - DNA, 86 bp, synthetic construct
  source 1..86: mol_type = other DNA
cgataagcttgatatcgaattcaaaaaaacagtcagggcgttgtccgctgtcacgactccataggaggaccgactcggtcccactt

SEQ ID NO: 51 - DNA, 45 bp, synthetic construct
  source 1..45: mol_type = other DNA
ctacacgacgctcttccgatctagccttcaacctgtatgccactg

SEQ ID NO: 52 - DNA, 44 bp, synthetic construct
  source 1..44: mol_type = other DNA
agacgtgtgctcttccgatctgcactctcgctcagattcttccc

SEQ ID NO: 53 - DNA, 48 bp, synthetic construct
  source 1..48: mol_type = other DNA
ctacacgacgctcttccgatctagtgtttacttgtcctatggctctct

SEQ ID NO: 54 - DNA, 46 bp, synthetic construct
  source 1..46: mol_type = other DNA
agacgtgtgctcttccgatctaattgactttcaggttgtcatttcc

SEQ ID NO: 55 - DNA, 46 bp, synthetic construct
  source 1..46: mol_type = other DNA
ctacacgacgctcttccgatctaggagtcgcctaaccctagagaag

SEQ ID NO: 56 - DNA, 40 bp, synthetic construct
  source 1..40: mol_type = other DNA
agacgtgtgctcttccgatctctccccaggtcccctcaaa

SEQ ID NO: 57 - DNA, 47 bp, synthetic construct
  source 1..47: mol_type = other DNA
ctacacgacgctcttccgatctatcctttgcttgtcagtgatactac

SEQ ID NO: 58 - DNA, 47 bp, synthetic construct
  source 1..47: mol_type = other DNA
agacgtgtgctcttccgatcttgtctcttcaacatacttgttttctc

SEQ ID NO: 59 - DNA, 47 bp, synthetic construct
  source 1..47: mol_type = other DNA
ctacacgacgctcttccgatcttatagcatagcaggatagcattcta

SEQ ID NO: 60 - DNA, 51 bp, synthetic construct
  source 1..51: mol_type = other DNA
agacgtgtgctcttccgatcttgatataatgtcaatttagagtataggact

SEQ ID NO: 61 - DNA, 46 bp, synthetic construct
  source 1..46: mol_type = other DNA
ctacacgacgctcttccgatctgttttcagagaatatgattgttgg

SEQ ID NO: 62 - DNA, 45 bp, synthetic construct
  source 1..45: mol_type = other DNA
agacgtgtgctcttccgatctcttcactttaccaccacatttcta

SEQ ID NO: 63 - DNA, 45 bp, synthetic construct
  source 1..45: mol_type = other DNA
ctacacgacgctcttccgatctttgctctctcttcctgtctccct

SEQ ID NO: 64 - DNA, 43 bp, synthetic construct
  source 1..43: mol_type = other DNA
agacgtgtgctcttccgatctccatcaggcatcaagaacaagg

SEQ ID NO: 65 - DNA, 41 bp, synthetic construct
  source 1..41: mol_type = other DNA
ctacacgacgctcttccgatctcggcacccaaaaggaggat

SEQ ID NO: 66 - DNA, 42 bp, synthetic construct
  source 1..42: mol_type = other DNA
agacgtgtgctcttccgatctcagggttcaggggcttcagag

SEQ ID NO: 67 - DNA, 43 bp, synthetic construct
  source 1..43: mol_type = other DNA
ctacacgacgctcttccgatctgcccacagccaagacagaaca

SEQ ID NO: 68 - DNA, 47 bp, synthetic construct
  source 1..47: mol_type = other DNA
agacgtgtgctcttccgatctcaccttgtaatgtgctctcaggaaac

SEQ ID NO: 69 - DNA, 41 bp, synthetic construct
  source 1..41: mol_type = other DNA
ctacacgacgctcttccgatctatgctcgccagcctgccac

SEQ ID NO: 70 - DNA, 45 bp, synthetic construct
  source 1..45: mol_type = other DNA
agacgtgtgctcttccgatctcctgtctcctctcatccctgttcc

SEQ ID NO: 71 - DNA, 43 bp, synthetic construct
  source 1..43: mol_type = other DNA
ctacacgacgctcttccgatctgaggtgtgagatgctcgccag

SEQ ID NO: 72 - DNA, 41 bp, synthetic construct
  source 1..41: mol_type = other DNA
agacgtgtgctcttccgatctgcaagccaggtgaccagcat

SEQ ID NO: 73 - DNA, 44 bp, synthetic construct
  source 1..44: mol_type = other DNA
ctacacgacgctcttccgatctgctacaactgacctctgtgccc

SEQ ID NO: 74 - DNA, 44 bp, synthetic construct
  source 1..44: mol_type = other DNA
agacgtgtgctcttccgatctctggttggaatgctgaagttgtc

SEQ ID NO: 75 - DNA, 45 bp, synthetic construct
  source 1..45: mol_type = other DNA
ctacacgacgctcttccgatctcccaaccctggagtttctgattc

SEQ ID NO: 76 - DNA, 46 bp, synthetic construct
  source 1..46: mol_type = other DNA
agacgtgtgctcttccgatctccacactgagaaggaggtgatgaga

SEQ ID NO: 77 - DNA, 43 bp, synthetic construct
  source 1..43: mol_type = other DNA
ctacacgacgctcttccgatctccaactgccttgatttgtgcc

SEQ ID NO: 78 - DNA, 51 bp, synthetic construct
  source 1..51: mol_type = other DNA
agacgtgtgctcttccgatcttgcttgattctcctttatagaattgttaca

SEQ ID NO: 79 - DNA, 46 bp, synthetic construct
  source 1..46: mol_type = other DNA
ctacacgacgctcttccgatctcacatttatttaggtgatgctgct

SEQ ID NO: 80 - DNA, 46 bp, synthetic construct
  source 1..46: mol_type = other DNA
agacgtgtgctcttccgatctagaaaaatgtgggatgattgtaaag

SEQ ID NO: 81 - DNA, 46 bp, synthetic construct
  source 1..46: mol_type = other DNA
ctacacgacgctcttccgatctttagaagataggagggctggaacg

SEQ ID NO: 82 - DNA, 41 bp, synthetic construct
  source 1..41: mol_type = other DNA
agacgtgtgctcttccgatctggcaagggctttttccacca

SEQ ID NO: 83 - DNA, 44 bp, synthetic construct
  source 1..44: mol_type = other DNA
ctacacgacgctcttccgatctgcatggcaaaatggcaatacat

SEQ ID NO: 84 - DNA, 41 bp, synthetic construct
  source 1..41: mol_type = other DNA
agacgtgtgctcttccgatcttgatgggtgtgctgggatgt

SEQ ID NO: 85 - DNA, 14 bp, synthetic construct
  source 1..14: mol_type = other DNA
ctgtctcttataca

SEQ ID NO: 86 - DNA, 80 bp, synthetic construct
  source 1..80: mol_type = other DNA
aatgatacggcgaccaccgagatctacactagatcgcnnnnnnnnnntcgtcggcagcgtcagatgtgtataagagacag

SEQ ID NO: 87 - DNA, 80 bp, synthetic construct
  source 1..80: mol_type = other DNA
aatgatacggcgaccaccgagatctacacctctctatnnnnnnnnnntcgtcggcagcgtcagatgtgtataagagacag

SEQ ID NO: 88 - DNA, 80 bp, synthetic construct
  source 1..80: mol_type = other DNA
aatgatacggcgaccaccgagatctacactatcctctnnnnnnnnnntcgtcggcagcgtcagatgtgtataagagacag

SEQ ID NO: 89 - DNA, 80 bp, synthetic construct
  source 1..80: mol_type = other DNA
aatgatacggcgaccaccgagatctacacagagtagannnnnnnnnntcgtcggcagcgtcagatgtgtataagagacag

SEQ ID NO: 90 - DNA, 80 bp, synthetic construct
  source 1..80: mol_type = other DNA
aatgatacggcgaccaccgagatctacacgtaaggagnnnnnnnnnntcgtcggcagcgtcagatgtgtataagagacag

SEQ ID NO: 91 - DNA, 80 bp, synthetic construct
  source 1..80: mol_type = other DNA
aatgatacggcgaccaccgagatctacacactgcatannnnnnnnnntcgtcggcagcgtcagatgtgtataagagacag

SEQ ID NO: 92 - DNA, 80 bp, synthetic construct
  source 1..80: mol_type = other DNA
aatgatacggcgaccaccgagatctacacaaggagtannnnnnnnnntcgtcggcagcgtcagatgtgtataagagacag

SEQ ID NO: 93 - DNA, 80 bp, synthetic construct
  source 1..80: mol_type = other DNA
aatgatacggcgaccaccgagatctacacctaagcctnnnnnnnnnntcgtcggcagcgtcagatgtgtataagagacag

SEQ ID NO: 94 - DNA, 29 bp, synthetic construct
  source 1..29: mol_type = other DNA
aatgatacggcgaccaccgagatctacac

SEQ ID NO: 95 - DNA, 53 bp, synthetic construct
  source 1..53: mol_type = other DNA
caagcagaagacggcatacgagatagcggaatgtgactggagttcagacgtgt

SEQ ID NO: 96 - DNA, 53 bp, synthetic construct
  source 1..53: mol_type = other DNA
caagcagaagacggcatacgagatgatcatgcgtgactggagttcagacgtgt

SEQ ID NO: 97 - DNA, 53 bp, synthetic construct
  source 1..53: mol_type = other DNA
caagcagaagacggcatacgagataagacggagtgactggagttcagacgtgt

SEQ ID NO: 98 - DNA, 53 bp, synthetic construct
  source 1..53: mol_type = other DNA
caagcagaagacggcatacgagatcgagtcctgtgactggagttcagacgtgt

SEQ ID NO: 99 - DNA, 53 bp, synthetic construct
  source 1..53: mol_type = other DNA
caagcagaagacggcatacgagattcctcagggtgactggagttcagacgtgt

SEQ ID NO: 100 - DNA, 53 bp, synthetic construct
  source 1..53: mol_type = other DNA
caagcagaagacggcatacgagatgtacggatgtgactggagttcagacgtgt

SEQ ID NO: 101 - DNA, 53 bp, synthetic construct
  source 1..53: mol_type = other DNA
caagcagaagacggcatacgagatcatctctcgtgactggagttcagacgtgt

SEQ ID NO: 102 - DNA, 53 bp, synthetic construct
  source 1..53: mol_type = other DNA
caagcagaagacggcatacgagatgtcggagcgtgactggagttcagacgtgt

SEQ ID NO: 103 - DNA, 53 bp, synthetic construct
  source 1..53: mol_type = other DNA
caagcagaagacggcatacgagatacggagaagtgactggagttcagacgtgt

SEQ ID NO: 104 - DNA, 53 bp, synthetic construct
  source 1..53: mol_type = other DNA
caagcagaagacggcatacgagataggagatggtgactggagttcagacgtgt

SEQ ID NO: 105 - DNA, 53 bp, synthetic construct
  source 1..53: mol_type = other DNA
caagcagaagacggcatacgagatagtactcggtgactggagttcagacgtgt

SEQ ID NO: 106 - DNA, 53 bp, synthetic construct
  source 1..53: mol_type = other DNA
caagcagaagacggcatacgagatggactctagtgactggagttcagacgtgt

SEQ ID NO: 107 - DNA, 76 bp, synthetic construct
  source 1..76: mol_type = other DNA
gttttagagctagaaatagcaagttaaaataaggctagtccgttatcaacttgaaaaagtgggaccgagtcggtcc

SEQ ID NO: 108 - DNA, 20 bp, synthetic construct
  source 1..20: mol_type = other DNA
ggcactgcggctggaggtgg

SEQ ID NO: 109 - DNA, 23 bp, synthetic construct
  source 1..23: mol_type = other DNA
acccccagagtcgtcctccagcc

SEQ ID NO: 110 - DNA, 20 bp, synthetic construct
  source 1..20: mol_type = other DNA
ggcactgcggctggaggtgg

SEQ ID NO: 111 - DNA, 32 bp, synthetic construct
  source 1..32: mol_type = other DNA
tccgctttaacccccagagtcgtcctccagcc

SEQ ID NO: 112 - DNA, 20 bp, synthetic construct
  source 1..20: mol_type = other DNA
ggcactgcggctggaggtgg

SEQ ID NO: 113 - DNA, 36 bp, synthetic construct
  source 1..36: mol_type = other DNA
agtctccgctttaacccccagagtcgtcctccagcc

SEQ ID NO: 114 - DNA, 20 bp, synthetic construct
  source 1..20: mol_type = other DNA
ggcactgcggctggaggtgg

SEQ ID NO: 115 - DNA, 27 bp, synthetic construct
  source 1..27: mol_type = other DNA
acccccagagtcgtgacacctccagcc

SEQ ID NO: 116 - DNA, 20 bp, synthetic construct
  source 1..20: mol_type = other DNA
ggcactgcggctggaggtgg

SEQ ID NO: 117 - DNA, 36 bp, synthetic construct
  source 1..36: mol_type = other DNA
tccgctttaacccccagagtcgtgacacctccagcc

SEQ ID NO: 118 - DNA, 20 bp, synthetic construct
  source 1..20: mol_type = other DNA
ggcactgcggctggaggtgg

SEQ ID NO: 119 - DNA, 40 bp, synthetic construct
  source 1..40: mol_type = other DNA
agtctccgctttaacccccagagtcgtgacacctccagcc

SEQ ID NO: 120 - DNA, 20 bp, synthetic construct
  source 1..20: mol_type = other DNA
ggcactgcggctggaggtgg

SEQ ID NO: 121 - DNA, 36 bp, synthetic construct
  source 1..36: mol_type = other DNA
acccccagagtcgtgacagcggacaaccctccagcc

SEQ ID NO: 122 - DNA, 20 bp, synthetic construct
  source 1..20: mol_type = other DNA
ggcactgcggctggaggtgg

SEQ ID NO: 123 - DNA, 45 bp, synthetic construct
  source 1..45: mol_type = other DNA
tccgctttaacccccagagtcgtgacagcggacaaccctccagcc

SEQ ID NO: 124 - DNA, 20 bp, synthetic construct
  source 1..20: mol_type = other DNA
ggcactgcggctggaggtgg

SEQ ID NO: 125 - DNA, 49 bp, synthetic construct
  source 1..49: mol_type = other DNA
agtctccgctttaacccccagagtcgtgacagcggacaaccctccagcc

SEQ ID NO: 126 - DNA, 20 bp, synthetic construct
  source 1..20: mol_type = other DNA
ggcactgcggctggaggtgg

SEQ ID NO: 127 - DNA, 36 bp, synthetic construct; identical to SEQ ID NO: 121
  source 1..36: mol_type = other DNA

SEQ ID NO: 128 - DNA, 20 bp, synthetic construct
  source 1..20: mol_type = other DNA
gaccccctccaccccgcctc

SEQ ID NO: 129 - DNA, 38 bp, synthetic construct
  source 1..38: mol_type = other DNA
gcccggaggagtcgtgacagcggacaacgcggggtgga

SEQ ID NO: 130 - DNA, 20 bp, synthetic construct
  source 1..20: mol_type = other DNA
ggcactgcggctggaggtgg

SEQ ID NO: 131 - DNA, 36 bp, synthetic construct; identical to SEQ ID NO: 121
  source 1..36: mol_type = other DNA

SEQ ID NO: 132 - DNA, 20 bp, synthetic construct
  source 1..20: mol_type = other DNA
ggcactgcggctggaggtgg

SEQ ID NO: 133 - DNA, 81 bp, synthetic construct
  source 1..81: mol_type = other DNA
acccccagagtcgtgacagcggacaaccctccagccaatctatacgcggttctatctagttacgcgttaaaccaactagaa

SEQ ID NO: 134 - DNA, 21 bp, synthetic construct
  source 1..21: mol_type = other DNA
gagcagcagcggcggcaacag

SEQ ID NO: 135 - DNA, 38 bp, synthetic construct
  source 1..38: mol_type = other DNA
tgccgctggagtcgtgacagcggacaacttgccgccgc

SEQ ID NO: 136 - DNA, 21 bp, synthetic construct
  source 1..21: mol_type = other DNA
gagcagcagcggcggcaacag

SEQ ID NO: 137 - DNA, 83 bp, synthetic construct
  source 1..83: mol_type = other DNA
tgccgctggagtcgtgacagcggacaacttgccgccgcttatataccgcggttctatctagttacgcgttaaaccaactagaa

SEQ ID NO: 138 - DNA, 21 bp, synthetic construct
  source 1..21: mol_type = other DNA
gaccattaaagaaaatatcat

SEQ ID NO: 139 - DNA, 27 bp, synthetic construct
  source 1..27: mol_type = other DNA
aaacaccaaagatgatattttctttaa

SEQ ID NO: 140 - DNA, 21 bp, synthetic construct
  source 1..21: mol_type = other DNA
gaccattaaagaaaatatcat

SEQ ID NO: 141 - DNA, 72 bp, synthetic construct
  source 1..72: mol_type = other DNA
aaacaccaaagatgatattttctttaaacactctgcgcggttctatctagttacgcgttaaaccaactagaa

SEQ ID NO: 142 - DNA, 21 bp, synthetic construct
  source 1..21: mol_type = other DNA
gatccttccagtcagggccat

SEQ ID NO: 143 - DNA, 31 bp, synthetic construct
  source 1..31: mol_type = other DNA
acctgaaccgtatatcctatggccctgactg

SEQ ID NO: 144 - DNA, 21 bp, synthetic construct
  source 1..21: mol_type = other DNA
gatccttccagtcagggccat

SEQ ID NO: 145 - DNA, 76 bp, synthetic construct
  source 1..76: mol_type = other DNA
acctgaaccgtatatcctatggccctgactgtccctatacgcggttctatctagttacgcgttaaaccaactagaa

SEQ ID NO: 146 - DNA, 21 bp, synthetic construct
  source 1..21: mol_type = other DNA
gccctaatgattttgacttca

SEQ ID NO: 147 - DNA, 29 bp, synthetic construct
  source 1..29: mol_type = other DNA
tcccagttacggtgaagtcaaaatcatta

SEQ ID NO: 148 - DNA, 21 bp, synthetic construct
  source 1..21: mol_type = other DNA
gccctaatgattttgacttca

SEQ ID NO: 149 - DNA, 74 bp, synthetic construct
  source 1..74: mol_type = other DNA
tcccagttacggtgaagtcaaaatcattaatcagttacgcggttctatctagttacgcgttaaaccaactagaa

SEQ ID NO: 150 - DNA, 21 bp, synthetic construct
  source 1..21: mol_type = other DNA
gaccattaaagaaaatatcat

SEQ ID NO: 151 - DNA, 40 bp, synthetic construct
  source 1..40: mol_type = other DNA
accgatggagtcgtgacagcggacaacatattttctttaa

SEQ ID NO: 152 - DNA, 21 bp, synthetic construct
  source 1..21: mol_type = other DNA
gaccattaaagaaaatatcat

SEQ ID NO: 153 - DNA, 85 bp, synthetic construct
  source 1..85: mol_type = other DNA
accgatggagtcgtgacagcggacaacatattttctttaacccaaacccgcggttctatctagttacgcgttaaaccaactagaa

SEQ ID NO: 154 - DNA, 21 bp, synthetic construct
  source 1..21: mol_type = other DNA
gatccttccagtcagggccat

SEQ ID NO: 155 - DNA, 37 bp, synthetic construct
  source 1..37: mol_type = other DNA
tcctatggagtcgtgacagcggacaacgccctgactg

SEQ ID NO: 156 - DNA, 21 bp, synthetic construct
  source 1..21: mol_type = other DNA
gatccttccagtcagggccat

SEQ ID NO: 157 - DNA, 82 bp, synthetic construct
  source 1..82: mol_type = other DNA
tcctatggagtcgtgacagcggacaacgccctgactgcctacaagcgcggttctatctagttacgcgttaaaccaactagaa

SEQ ID NO: 158 - DNA, 21 bp, synthetic construct
  source 1..21: mol_type = other DNA
gccctaatgattttgacttca

SEQ ID NO: 159 - DNA, 49 bp, synthetic construct
  source 1..49: mol_type = other DNA
tcccagttaccatgagagtcgtgacagcggacaacagtcaaaatcatta

SEQ ID NO: 160 - DNA, 21 bp, synthetic construct
  source 1..21: mol_type = other DNA
gccctaatgattttgacttca

SEQ ID NO: 161 - DNA, 94 bp, synthetic construct
  source 1..94: mol_type = other DNA
tcccagttaccatgagagtcgtgacagcggacaacagtcaaaatcattattatatcccgcggttctatctagttacgcgttaaaccaactagaa

SEQ ID NO: 162 - DNA, 20 bp, synthetic construct
  source 1..20: mol_type = other DNA
ggcactgcggctggaggtgg

SEQ ID NO: 163 - DNA, 18 bp, synthetic construct
  source 1..18: mol_type = other DNA
taaccccaacctccagcc

SEQ ID NO: 164 - DNA, 20 bp, synthetic construct
  source 1..20: mol_type = other DNA
ggcactgcggctggaggtgg

SEQ ID NO: 165 - DNA, 27 bp, synthetic construct
  source 1..27: mol_type = other DNA
tctccgctttaaccccaacctccagcc

SEQ ID NO: 166 - DNA, 20 bp, synthetic construct
  source 1..20: mol_type = other DNA
ggcactgcggctggaggtgg

SEQ ID NO: 167 - DNA, 31 bp, synthetic construct
  source 1..31: mol_type = other DNA
agagtctccgctttaaccccaacctccagcc

SEQ ID NO: 168 - DNA, 20 bp, synthetic construct
  source 1..20: mol_type = other DNA
ggcactgcggctggaggtgg

SEQ ID NO: 169 - DNA, 16 bp, synthetic construct
  source 1..16: mol_type = other DNA
aaccccccctccagcc

SEQ ID NO: 170 - DNA, 20 bp, synthetic construct
  source 1..20: mol_type = other DNA
ggcactgcggctggaggtgg

SEQ ID NO: 171 - DNA, 26 bp, synthetic construct
  source 1..26: mol_type = other DNA
tctccgctttaaccccccctccagcc

SEQ ID NO: 172 - DNA, 20 bp, synthetic construct
  source 1..20: mol_type = other DNA
ggcactgcggctggaggtgg

SEQ ID NO: 173 - DNA, 30 bp, synthetic construct
  source 1..30: mol_type = other DNA
agagtctccgctttaaccccccctccagcc

SEQ ID NO: 174 - DNA, 20 bp, synthetic construct
  source 1..20: mol_type = other DNA
ggcactgcggctggaggtgg

SEQ ID NO: 175 - DNA, 18 bp, synthetic construct
  source 1..18: mol_type = other DNA
aacccccaacctccagcc

SEQ ID NO: 176 - DNA, 20 bp, Synthetic construct
  source 1..20: mol_type = other DNA
ggcactgcggctggaggtgg

SEQ ID NO: 177 - DNA, 28 bp, synthetic construct
  source 1..28: mol_type = other DNA
tctccgctttaacccccaacctccagcc

SEQ ID NO: 178 - DNA, 20 bp, synthetic construct
  source 1..20: mol_type = other DNA
ggcactgcggctggaggtgg

SEQ ID NO: 179 - DNA, 32 bp, synthetic construct
  source 1..32: mol_type = other DNA
agagtctccgctttaacccccaacctccagcc

SEQ ID NO: 180 - DNA, 20 bp, synthetic construct
  source 1..20: mol_type = other DNA
gagtccgagcagaagaagaa

SEQ ID NO: 181 - DNA, 42 bp, synthetic construct
  source 1..42: mol_type = other DNA
tcctccacctgagtcgtgacagcggacaaccctccggccgca

SEQ ID NO: 182 - DNA, 21 bp, synthetic construct
  source 1..21: mol_type = other DNA
gaccattaaagaaaatatcat

SEQ ID NO: 183 - DNA, 21 bp, synthetic construct
  source 1..21: mol_type = other DNA
gatccttccagtcagggccat

SEQ ID NO: 184 - DNA, 21 bp, synthetic construct
  source 1..21: mol_type = other DNA
gccctaatgattttgacttca

SEQ ID NO: 185 - RNA, 148 nt, synthetic construct
  source 1..148: mol_type = other RNA
tgcactgcggccggaggagggttttagagctagaaatagcaagttaaaataaggctagtccgttatcaacttgaaaaagtgggaccgagtcggtccttactttccatcctccacctgagtcgtgacagcggacaaccctccggccgca

SEQ ID NO: 186 - RNA, 145 nt, synthetic construct
  source 1..145: mol_type = other RNA
ggcactgcggctggaggtgggttttagagctagaaatagcaagttaaaataaggctagtccgttatcaacttgaaaaagtgggaccgagtcggtccagtctccgctttaacccccagagtcgtgacagcggacaaccctccagcc

SEQ ID NO: 187 - RNA, 146 nt, synthetic construct
  source 1..146: mol_type = other RNA
agcagcagcggcggcaacaggttttagagctagaaatagcaagttaaaataaggctagtccgttatcaacttgaaaaagtgggaccgagtcggtccggtggccgctgttgccgctggagtcgtgacagcggacaacttgccgccgc

SEQ ID NO: 188 - RNA, 134 nt, synthetic construct
  source 1..134: mol_type = other RNA
gaccccctccaccccgcctcgttttagagctagaaatagcaagttaaaataaggctagtccgttatcaacttgaaaaagtgggaccgagtcggtccgcccggaggagtcgtgacagcggacaacgcggggtgga

SEQ ID NO: 189 - RNA, 133 nt, synthetic construct
  source 1..133: mol_type = other RNA
atccttccagtcagggccatgttttagagctagaaatagcaagttaaaataaggctagtccgttatcaacttgaaaaagtgggaccgagtcggtcctcctatggagtcgtgacagcggacaacgccctgactg

SEQ ID NO: 190 - RNA, 128 nt, synthetic construct
  source 1..128: mol_type = other RNA
gatccttccagtcagggccatgttttagagctagaaatagcaagttaaaataaggctagtccgttatcaacttgaaaaagtgggaccgagtcggtccacctgaaccgtatatcctatggccctgactg

SEQ ID NO: 191 - RNA, 145 nt, synthetic construct
  source 1..145: mol_type = other RNA
ccctaatgattttgacttcagttttagagctagaaatagcaagttaaaataaggctagtccgttatcaacttgaaaaagtgggaccgagtcggtcctcccagttaccatgagagtcgtgacagcggacaacagtcaaaatcatta

SEQ ID NO: 192 - RNA, 125 nt, synthetic construct
  source 1..125: mol_type = other RNA
ccctaatgattttgacttcagttttagagctagaaatagcaagttaaaataaggctagtccgttatcaacttgaaaaagtgggaccgagtcggtcctcccagttacggtgaagtcaaaatcatta

SEQ ID NO: 193 - RNA, 136 nt, synthetic construct
  source 1..136: mol_type = other RNA
accattaaagaaaatatcatgttttagagctagaaatagcaagttaaaataaggctagtccgttatcaacttgaaaaagtgggaccgagtcggtccaccgatggagtcgtgacagcggacaacatattttctttaa

SEQ ID NO: 194 - RNA, 123 nt, synthetic construct
  source 1..123: mol_type = other RNA
accattaaagaaaatatcatgttttagagctagaaatagcaagttaaaataaggctagtccgttatcaacttgaaaaagtgggaccgagtcggtccaaacaccaaagatgatattttctttaa

SEQ ID NO: 195 - RNA, 100 nt, synthetic construct
  source 1..100: mol_type = other RNA
  modified_base 1..3: mod_base = OTHER; note = The residues at these positions are 2-O-methylated ribonucleotides
  modified_base 97..99: mod_base = OTHER; note = The residues at these positions are 2-O-methylated ribonucleotides
  misc_feature 1..4: note = There exists a Phosphorothioate linkage between these residues
  misc_feature 97..100: note = There exists a Phosphorothioate linkage between these residues
atccttccagtcagggccatgttttagagctagaaatagcaagttaaaataaggctagtccgttatcaacttgaaaaagtggcaccgagtcggtgctttt

SEQ ID NO: 196 - RNA, 100 nt, synthetic construct
  source 1..100: mol_type = other RNA
  modified_base 1..3: mod_base = OTHER; note = The residues at these positions are 2-O-methylated ribonucleotides
  modified_base 97..98: mod_base = OTHER; note = The residues at these positions are 2-O-methylated ribonucleotides
  misc_feature 1..4: note = There exists a Phosphorothioate linkage between these residues
  misc_feature 97..100: note = There exists a Phosphorothioate linkage between these residues
ccctaatgattttgacttcagttttagagctagaaatagcaagttaaaataaggctagtccgttatcaacttgaaaaagtggcaccgagtcggtgctttt

SEQ ID NO: 197 - RNA, 100 nt, synthetic construct
  source 1..100: mol_type = other RNA
  modified_base 1..3: mod_base = OTHER; note = The residues at these positions are 2-O-methylated ribonucleotides
  modified_base 97..99: mod_base = OTHER; note = The residues at these positions are 2-O-methylated ribonucleotides
  misc_feature 1..4: note = There exists a Phosphorothioate linkage between these residues
  misc_feature 97..100: note = There exists a Phosphorothioate linkage between these residues
accattaaagaaaatatcatgttttagagctagaaatagcaagttaaaataaggctagtccgttatcaacttgaaaaagtggcaccgagtcggtgctttt

SEQ ID NO: 198 - DNA, 58 bp, synthetic construct
  source 1..58: mol_type = other DNA
gtggaaaggacgaaacaccggcactgcggctggaggtgggttttagagctagaaatag

SEQ ID NO: 199 - DNA, 75 bp, synthetic construct
  source 1..75: mol_type = other DNA
cggtatcgataagcttgatatcgaattcaaaaaaggctggaggacgactctgggggtggaccgactcggtcccac

SEQ ID NO: 200 - DNA, 83 bp, synthetic construct
  source 1..83: mol_type = other DNA
ggtatcgataagcttgatatcgaattcaaaaaaggctggaggacgactctgggggttaaagcggaggaccgactcggtcccac

SEQ ID NO: 201 - DNA, 87 bp, synthetic construct
  source 1..87: mol_type = other DNA
ggtatcgataagcttgatatcgaattcaaaaaaggctggaggacgactctgggggttaaagcggagactggaccgactcggtcccac

SEQ ID NO: 202 - DNA, 79 bp, synthetic construct
  source 1..79: mol_type = other DNA
cggtatcgataagcttgatatcgaattcaaaaaaggctggaggtgtcacgactctgggggtggaccgactcggtcccac

SEQ ID NO: 203 - DNA, 87 bp, synthetic construct
  source 1..87: mol_type = other DNA
ggtatcgataagcttgatatcgaattcaaaaaaggctggaggtgtcacgactctgggggttaaagcggaggaccgactcggtcccac

SEQ ID NO: 204 - DNA, 91 bp, synthetic construct
  source 1..91: mol_type = other DNA
ggtatcgataagcttgatatcgaattcaaaaaaggctggaggtgtcacgactctgggggttaaagcggagactggaccgactcggtcccac

SEQ ID NO: 205 - DNA, 83 bp, synthetic construct
  source 1..83: mol_type = other DNA
tcgataagcttgatatcgaattcaaaaaaggctggagggttgtccgctgtcacgactctgggggtggaccgactcggtcccac

SEQ ID NO: 206 - DNA, 93 bp, synthetic construct
  source 1..93: mol_type = other DNA
atcgataagcttgatatcgaattcaaaaaaggctggagggttgtccgctgtcacgactctgggggttaaagcggaggaccgactcggtcccac

SEQ ID NO: 207 - DNA, 97 bp, synthetic construct
  source 1..97: mol_type = other DNA
atcgataagcttgatatcgaattcaaaaaaggctggagggttgtccgctgtcacgactctgggggttaaagcggagactggaccgactcggtcccac

SEQ ID NO: 208 - DNA, 57 bp, synthetic construct
  source 1..57: mol_type = other DNA
tggaaaggacgaaacaccgaccccctccaccccgcctcgttttagagctagaaatag

SEQ ID NO: 209 - DNA, 86 bp, synthetic construct
  source 1..86: mol_type = other DNA
tcgataagcttgatatcgaattcaaaaaatccaccccgcgttgtccgctgtcacgactcctccgggcggaccgactcggtcccact

SEQ ID NO: 210 - DNA, 60 bp, synthetic construct
  source 1..60: mol_type = other DNA
gtggaaaggacgaaacaccgagcagcagcggcggcaacaggttttagagctagaaatagc

SEQ ID NO: 211 - DNA, 86 bp, synthetic construct
  source 1..86: mol_type = other DNA
tcgataagcttgatatcgaattcaaaaaagcggcggcaagttgtccgctgtcacgactccagcggcaggaccgactcggtcccact

SEQ ID NO: 212 - DNA, 67 bp, synthetic construct
  source 1..67: mol_type = other DNA
tcgataagcttgatatcgaattcaaaaaatccaccccgccgccgggcgcggaccgactcggtcccac

SEQ ID NO: 213 - DNA, 74 bp, synthetic construct
  source 1..74: mol_type = other DNA
tcgataagcttgatatcgaattcaaaaaatccaccccgccgccgggcgcgggctccggaccgactcggtcccac

SEQ ID NO: 214 - DNA, 79 bp, synthetic construct
  source 1..79: mol_type = other DNA
tcgataagcttgatatcgaattcaaaaaatccaccccgccgccgggcgcgggctccggcccggaccgactcggtcccac

SEQ ID NO: 215 - DNA, 67 bp, synthetic construct
  source 1..67: mol_type = other DNA
tcgataagcttgatatcgaattcaaaaaatccaccccgccccgggcgcggaccgactcggtcccact

SEQ ID NO: 216 - DNA, 75 bp, synthetic construct
  source 1..75: mol_type = other DNA
atcgataagcttgatatcgaattcaaaaaatccaccccgccccgggcgcgggctccggaccgactcggtcccact

SEQ ID NO: 217 - DNA, 80 bp, synthetic construct
  source 1..80: mol_type = other DNA
atcgataagcttgatatcgaattcaaaaaatccaccccgccccgggcgcgggctccggcccggaccgactcggtcccact

SEQ ID NO: 218 - DNA, 67 bp, synthetic construct
  source 1..67: mol_type = other DNA
tcgataagcttgatatcgaattcaaaaaatccaccccgccgtccgggcggaccgactcggtcccact

SEQ ID NO: 219 - DNA, 76 bp, synthetic construct
  source 1..76: mol_type = other DNA
atcgataagcttgatatcgaattcaaaaaatccaccccgccgtccgggcgcgggctccggaccgactcggtcccac

SEQ ID NO: 220 - DNA, 82 bp, synthetic construct
  source 1..82: mol_type = other DNA
atcgataagcttgatatcgaattcaaaaaatccaccccgccgtccgggcgcgggctccggccggaccgactcggtcccactt

SEQ ID NO: 221 - DNA, 59 bp, synthetic construct
  source 1..59: mol_type = other DNA
gtggaaaggacgaaacaccgtgcactgcggccggaggagggttttagagctagaaatag

SEQ ID NO: 222 - DNA, 89 bp, synthetic construct
  source 1..89: mol_type = other DNA
tcgataagcttgatatcgaattcaaaaaatgcggccggagggttgtccgctgtcacgactcaggtggaggaggaccgactcggtcccac

SEQ ID NO: 223 - DNA, 388 bp, synthetic construct
  source 1..388: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcactgcggctggaggtgggttttagagctagaaatagcaagttaaaataaggctagtccgttatcaacttgaaaaagtgggaccgagtcggtccacccccagagtcgtgacagcggacaaccctccagccttttttt

(19) United States
(12) Patent Application Publication
(10) Pub. No.: US 2026/0250743 A1
(43) Pub. Date: Aug. 27, 2026

(54) COMPOSITIONS AND METHODS OF PRIME EDITOR GUIDE RNA/SEQUENCING TAG COMPLEXES

(71) Applicant: UNIVERSITY OF MASSACHUSETTS, Westborough, MA (US)

(72) Inventors: Scot Wolfe, Winchester, MA (US); Wen Xue, Natick, MA (US); Shun-Qing Liang, Shrewsbury, MA (US); Pengpeng Liu, Worcester, MA (US)

(21) Appl. No.: 18/854,470

(22) PCT Filed: Apr. 6, 2023
(86) PCT No.: PCT/US2023/017700

§ 371 Date: Oct. 4, 2024

Related U.S. Application Data
(60) Provisional application No. 63/328,076, filed on Apr. 6, 2022.

(51) Int. Cl. C12Q 1/6816; C12N 9/12; C12N 9/22; C12N 15/10; C12N 15/11; C12Q 1/6883

(52) U.S. Cl. CPC C12Q 1/6816; C12N 9/1241; C12N 9/1276; C12N 9/226; C12N 15/1065; C12N 15/11; C12Q 1/6883; C12Y 207/07049; C12N 2310/20; C12Q 2600/156

(57) ABSTRACT

The present invention is related to the field of gene and cell therapy. In particular, to the clinical application of CRISPR-mediated gene and cell therapy. Herein, compositions and methods providing the ability to identify and quantitate editing at on-target and off-target prime editor recognition sites are disclosed. These compositions include prime editors working in tandem with pegRNAs associated with an amplification tag element that is inserted at a genomic prime editor recognition site. The method contemplates a polymerase chain reaction amplification of these genomic target sites that are then subjected to sequencing to determine whether the genomic prime editor recognition site is on-target or off-target.

### STATEMENT REGARDING FEDERALLY SPONSORED RESEARCH OR DEVELOPMENT

This invention was made with government support under grant nos. HL137167, HL147367, HL158506 and TR002668 awarded by the National Institutes of Health. The Government has certain rights in the invention.

### FIELD OF THE INVENTION

The present invention is related to the field of gene and cell therapy. In particular, to the clinical application of CRISPR-mediated gene and cell therapy. Herein, compositions and methods providing the ability to identify and quantitate editing activity at on-target and off-target prime editor recognition sites are disclosed. These compositions include prime editors working in tandem with pegRNAs associated with an amplification tag element that is inserted at a genomic prime editor recognition site. The method contemplates a polymerase chain reaction amplification of these genomic target sites that are then subjected to sequencing to determine the location of prime editor recognition sites-both on-target or off-target sites-within the genome and the relative activity of editing at these sites.

### BACKGROUND

Correction of genetic mutations in vivo has broad potential therapeutic application for a range of human genetic diseases. Prime editors (PE) composed of a Cas9 nickase and engineered reverse transcriptase have been reported to make nucleotide changes, sequence insertions and deletions. Anzalone, A. V. et al. Nature 576, 149-157 (2019)); see also FIG. 16. PE technology does not induce double-stranded DNA breaks and does not require a donor DNA template in conjunction with homology directed repair to introduce precise sequence changes into the genome. The ability to precisely correct pathogenic mutations makes prime editors a useful tool to perform somatic genome editing. Unlike conventional base editing systems, prime editors do not suffer from the challenges of bystander base conversion, which may provide advantages in some sequence contexts.

Recent improvements in the prime editor and pegRNA frameworks have been reported to increase the efficiency of prime editing in cells. Liu, P. et al. Nat Commun 12, 2121 (2021); Chen, P. J. et al. Cell 184, 5635-5652.e29 (2021); and Nelson, J. W. et al. Nat Biotechnol 1-9 (2021). In vivo delivery approaches are being investigated to correct disease mutations in mammalian systems with the long-term goal of translating this technology into therapeutic application. Liu, P. et al. Nat Commun 12, 2121 (2021); Jang H, et. al. Nat Biomed Eng. 2021 Aug. 26; and Zheng, C. et al. Mol Ther (2022).

Despite these improvements there is still a need in the art to identify sites of PE-induced off-target editing genome-wide to provide a framework for the development of rational approaches to reduce and/or prevent unwanted damage to the genome. Such a methodology for evaluating the safety of prime editing systems plays a role in the therapeutic advancement of this genome editing technology.

### SUMMARY OF THE INVENTION

The present invention is related to the field of gene and cell therapy. In particular, to the clinical application of CRISPR-mediated gene and cell therapy. Herein, compositions and methods providing the ability to identify and quantitate editing at on-target and off-target prime editor recognition sites are disclosed. These compositions include prime editors working in tandem with pegRNAs associated with an amplification tag element that is inserted at each genomic prime editor recognition site. The method contemplates a polymerase chain reaction amplification of these genomic target sites that are then subjected to sequencing to determine the genomic location of on-target or off-target prime editor recognition sites and to quantitate the editing activity at each of these recognition sites.

In one embodiment, the present invention contemplates a method, comprising: a) providing: i) an oligonucleotide sequence or a genomic sequence comprising a target sequence; ii) a composition comprising a Cas9 nickase (nCas9) protein and a reverse transcriptase protein; iii) a prime editor guide ribonucleic acid (pegRNA) that is complexed to the Cas9 nickase comprising a spacer sequence that is at least partially complementary to said target sequence and an amplification tag element; iv) a Tn5 transposase enzyme; and v) a universal adaptor; b) hybridizing the spacer sequence to said target sequence; c) nicking said target sequence with said nCas9 protein to create a free 3′ DNA end; d) incorporating said amplification tag element into said free 3′ DNA end with said reverse transcriptase; e) incorporating the universal adaptor into said oligonucleotide sequence or said genomic sequence with the Tn5 transposase enzyme; f) amplifying said oligonucleotide sequence or said genomic sequence between said amplification tag element and said universal adaptor to create a plurality of amplicons; and g) sequencing said plurality of amplicons to identify: i) an on-target prime editor recognition sequence when the spacer sequence is fully-cognate with the target sequence; or ii) an off-target prime editor recognition sequence when the spacer sequence is near-cognate with the target sequence. In one embodiment, the sequencing further determines prime editor editing efficiency at the on-target or off-target prime editor recognition sites. In one embodiment, the recognition sequence comprises a protospacer adjacent motif (PAM) sequence that is cognate or near-cognate with the nCas9 protein. In one embodiment, the nCas9 nickase protein and reverse transcriptase protein are a fusion protein. In one embodiment, the pegRNA further comprises a reverse transcriptase template. In one embodiment, the reverse transcriptase template comprises the amplification tag element. In one embodiment, the amplification tag element is SEQ ID NO: 1. In one embodiment, the amplifying further comprises a first amplification primer. In one embodiment, the first amplification primer is a reverse primer. In one embodiment, the reverse primer is complementary to the amplification tag element. In one embodiment, the sequencing further comprises a second amplification primer. In one embodiment, the second amplification primer is a forward primer. In one embodiment, the forward primer is complementary to the universal adaptor. In one embodiment, the universal adaptor comprises at least one unique molecular identifier (UMI) sequence. In one embodiment, the universal adaptor comprises a barcode sequence. In one embodiment, the second amplification primer is an i5primer. In one embodiment, the target sequence is a gene edited target sequence. In one embodiment, the target sequence is associated with a pathological medical condition. In one embodiment, the pathological medical condition is a genetic disease or disorder.

In one embodiment, the present invention contemplates a composition comprising a Cas9 nickase (nCas9) protein and a prime editor guide ribonucleic acid (pegRNA) encoding an amplification tag element. In one embodiment, the pegRNA further comprises a spacer sequence that is at least partially complementary to a prime editor recognition sequence. In one embodiment, the composition further comprises a reverse transcriptase protein. In one embodiment, the nCas9 protein and the reverse transcriptase protein are a fusion protein. In one embodiment, the pegRNA further comprises a reverse transcriptase template sequence. In one embodiment, the reverse transcriptase template sequence comprises the amplification tag element. In one embodiment, the amplification tag element is SEQ ID NO:1.

In one embodiment, the present invention contemplates a kit, comprising: a) a first container comprising a Cas9 nickase (nCas9) protein and a reverse transcriptase; and b) a second container comprising a prime editor guide ribonucleic acid (pegRNA) encoding a spacer sequence that is at least partially complementary to a prime editor recognition sequence and an amplification tag element. In one embodiment, the kit further comprises a third container comprising a control pegRNA. In one embodiment, the kit further comprises a fourth container comprising a primer set configured to hybridize with a control genomic region. In one embodiment, the kit further comprises a fifth container comprising a primer configured to hybridize with the amplification tag element. In one embodiment, the kit further comprises instructional materials for the use of the reagents in the performance of PE-tag and the generation of sequencing libraries. In one embodiment, the kit further comprises instructional materials that provide design parameters to construct a custom pegRNA. In one embodiment, the kit further comprises instructional materials to provide design parameters to construct locus-specific primers. In one embodiment, the kit further comprises instructional materials for the use of PE-tag sequence analysis software.

In one embodiment, the present invention contemplates a method, comprising: a) providing: i) a biological sample comprising an oligonucleotide sequence or a genomic sequence encoding a mutated target sequence; ii) a composition comprising a Cas9 nickase (nCas9) protein and a reverse transcriptase protein; iii) a pegRNA that is complexed to the Cas9 nickase comprising a spacer sequence that is at least partially complementary to said mutated target sequence and an amplification tag element; iv) a Tn5 transposase enzyme; and v) a universal adaptor; b) hybridizing the pegRNA spacer sequence to said mutated target sequence; c) nicking said oligonucleotide sequence or genomic sequence with said nCas9 protein to create a free 3′ DNA end; d) incorporating said amplification tag element into said free 3′ DNA end with said reverse transcriptase; e) appending the universal adaptor to said oligonucleotide sequence or said genomic sequence with the Tn5 transposase enzyme; f) amplifying said oligonucleotide sequence or said genomic sequence between said amplification tag element and said universal adaptor to create a plurality of amplicons; and g) sequencing said plurality of amplicons to identify the mutated target sequence as: i) an on-target gene editing site when the spacer sequence is fully cognate with the mutated target sequence; or ii) an off-target gene editing site when the spacer sequence is near-cognate with the mutated target sequence. In one embodiment, the biological sample is a biopsy. In one embodiment, the biological sample is derived from a patient exhibiting at least one symptom of a pathological medical condition. In one embodiment, the pathological medical condition is a genetic disease or disorder. In one embodiment, the method further comprises in vitro editing of the on-target gene editing site or the off-target gene editing site with a prime editor protein complex comprising a pegRNA with a reverse transcriptase template encoding a wild type target site to provide evidence of editing at a validated on-target site or a validated off-target site based on an amplification tag incorporation analysis. In one embodiment, the method further comprises administering said prime editor complex to the patient, wherein said prime editor complex contacts said validated on-target site or said validated off-target site and said at least one symptom of a pathological medical condition is reduced. In one embodiment, the sequencing further determines gene editing efficiency at the on-target site or off-target site. In one embodiment, the oligonucleotide sequence or genomic sequence comprises a protospacer adjacent motif (PAM) sequence that is cognate with the nCas9 protein. In one embodiment, the nCas9 nickase protein and reverse transcriptase protein are a fusion protein. In one embodiment, the amplification tag element is SEQ ID NO: 1. In one embodiment, the amplifying further comprises a first amplification primer. In one embodiment, the first amplification primer is a reverse primer. In one embodiment, the reverse primer is complementary to the amplification tag element. In one embodiment, the sequencing further comprises a second amplification primer. In one embodiment, the second amplification primer is a forward primer. In one embodiment, the forward primer is complementary to the universal adaptor. In one embodiment, the universal adaptor comprises at least one unique molecular identifier (UMI) sequence. In one embodiment, the universal adaptor comprises a barcode sequence. In one embodiment, the second amplification primer is an i5primer. In one embodiment, the mutated target sequence is associated with a pathological medical condition. In one embodiment, the pathological medical condition is a genetic disease or disorder.

In one embodiment, the present invention contemplates a method, comprising: a) providing: i) an oligonucleotide or genomic sequence comprising a prime editor recognition sequence; ii) a prime editor comprising a Cas9 nickase (nCas9) and a reverse transcriptase; iii) a prime editor guide ribonucleic acid (pegRNA) that is complexed to the Cas9 nickase comprising: an amplification tag element (e.g., a tag); a spacer sequence that is cognate with the prime editor recognition sequence; and a primer binding site; and iv) primers for amplification and sequencing the oligonucleotide or genomic sequence comprising the amplification tag element; b) contacting said prime editor to said oligonucleotide or genomic sequence based on complementarity between the spacer sequence within the pegRNA and a protospacer accessory motif sequence cognate or near-cognate with the prime editor; c) incorporating said amplification tag element into said prime editor recognition sequence via the nicking and reverse transcriptase activity of the prime editor to create an edited prime editor recognition sequence; d) appending a universal adaptor near said edited recognition sequence via Tn5 tagmentation; e) amplifying said edited genomic sequence between said amplification tag element and said universal adaptor; and g) sequencing all of the edited genomic sequences to determine the identity of the edited sequences within the genome and the efficiency of prime editing at the prime editor recognition sequence and off-target sites within the genome based on the number distinct UMI sequences present at each locus. In one embodiment, the prime editor comprises a fusion protein. In one embodiment, the prime editor comprises a plurality of separate proteins. In one embodiment, the amplification tag element is SEQ ID NO: 1. In one embodiment, the first amplification primer is a reverse primer (e.g., Tag primer R). In one embodiment, the reverse primer is complementary to the amplification tag element. In one embodiment, the method further provides a second amplification primer that is complementary to the universal adaptor. In one embodiment, the universal adaptor comprises at least one unique molecular identifier (UMI) sequence. In one embodiment, the universal adaptor comprises a library pooling barcode sequence. In one embodiment, the second amplification primer is an i5 primer. In one embodiment, the second amplification primer is a forward primer (e.g., primer F). In one embodiment, the amplifying further comprises generation of a plurality of amplicons. In one embodiment, the sequencing further comprises the plurality of amplicons. In one embodiment, the sequence targeted by the prime editor is associated with a pathological medical condition.

In one embodiment, the present invention contemplates methods for discovering prime editing sites within a genome, comprising: a) providing: i) an oligonucleotide sequence or a genomic sequence; ii) a composition comprising a Cas9 nickase (nCas9) protein and a reverse transcriptase protein; iii) a prime editor guide ribonucleic acid (pegRNA) that is complexed to the Cas9 nickase comprising an amplification tag element; iv) a Tn5 transposase enzyme; and v) a universal adaptor; b) nicking said oligonucleotide sequence or a genomic sequence with said nCas9 protein at functional sequences; c) incorporating said amplification tag element at said functional sequences with said pegRNA resulting in a gene edited sequence; d) tagmenting the universal adaptor into said oligonucleotide sequence or a genomic sequence with the Tn5 transposase enzyme; f) amplifying said gene edited sequence between said amplification tag element and said universal adaptor to create a plurality of amplicons; and g) sequencing said plurality of amplicons to determine functional off-target sites. In one embodiment, the sequencing further determines the identity of the gene edited sequences. In one embodiment, the sequencing further determines gene editing efficiency at the gene edited sequences based on the UMI sequence count at each locus. In one embodiment, the nCas9 nickase protein and reverse transcriptase protein are a fusion protein. In one embodiment, the pegRNA further comprises a reverse transcriptase template. In one embodiment, the reverse transcriptase template comprises the amplification tag element. In one embodiment, the amplification tag element is SEQ ID NO: 1. In one embodiment, the amplifying further comprises an amplification primer. In one embodiment, the amplification primer is a reverse primer. In one embodiment, the reverse primer is complementary to the amplification tag element. In one embodiment, the amplification further comprises a second amplification primer. In one embodiment, the second amplification primer is a forward primer. In one embodiment, the forward primer is complementary to the universal adaptor. In one embodiment, the universal adaptor comprises at least one unique molecular identifier (UMI) sequence. In one embodiment, the universal adaptor comprises a barcode sequence. In one embodiment, the amplification primer is an i5 primer. In one embodiment, the amplification incorporates elements for sequencing the amplicons. In one embodiment, one edited sequence is associated with a pathological medical condition.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer” sequence. The spacers are short segments of DNA from a virus and may serve as a ‘memory’ of past exposures to facilitate an adaptive defense against future invasions. Doudna et al. Genome editing. The new frontier of genome engineering with CRISPR-Cas9″ Science 346(6213):1258096 (2014).

As used herein, the term “Cas” or “CRISPR-associated (cas)” refers to genes often associated with CRISPR repeat-spacer arrays.

As used herein, the term “Cas9” refers to a nuclease from type II CRISPR systems, an enzyme specialized for generating double-strand breaks in DNA, with two active cutting sites (the HNH and RuvC domains), one for each strand of the double helix. tracrRNA and spacer RNA (crRNA) may be combined into a “single-guide RNA” (sgRNA) molecule that, mixed with Cas9, could find and cleave DNA targets through Watson-Crick pairing between the spacer (guide) sequence within the sgRNA and the target DNA sequence, Jinek et al. A programmable dual-RNA-guided DNA endonuclease in adaptive bacterial immunity” Science 337(6096):816-821 (2012).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337(6096):816-821 (2012) and Cong et al. Multiplex genome engineering using CRISPR/Cas systems” Science 339(6121):819-823 (2013).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “prime editor” (PE) refers to a fusion protein or separate proteins comprising a Cas9 nickase (nCas9) and a reverse transcriptase (RT) that function together to append a sequence to the genome or alter the local genomic sequence (i.e., for example, to change, insert, delete and/or insert a DNA sequence). Prime editors utilize a prime editing guide RNA (pegRNA), which is an sgRNA that contains a 3′ extension that provides a template for copying a defined sequence into the genome. After binding to its target site and forming an R-loop based on the encoded spacer sequence within the pegRNA, the prime editor nicks the non-template strand of the target site (the strand not annealed to the spacer region of the pegRNA). This nicked DNA strand anneals to a complementary region within the 3′ region of the pegRNA to serve as a primer that is extended by the reverse transcriptase based on the template within the pegRNA. In vitro, this sequence is added onto the genome as an extension. If this reaction is performed in vitro appropriate buffers and deoxynucleoside triphosphates (dNTPs) are necessary for the reverse transcriptase polymerization. In cells, this extension can be incorporated in genome by DNA repair enzymes based on sequence homology within the 3′ end of the extension to the local genomic sequence. A prime editor ribonucleoprotein (RNP) complex with a pegRNA can be utilized for editing in vitro or delivered to cells via electroporation or alternate method for editing. Anzalone, A. V. et al. Search-and-replace genome editing without double-strand breaks or donor DNA. Nature 576, 149-157 (2019). The prime editor may also constitute a Cas9 nuclease instead of a Cas9 nickase to facilitate the deletion and insertion of sequences when two different pegRNAs are employed. Jiang T, Zhang XO, Weng Z, Xue W. Deletion and replacement of long genomic sequences using prime editing. Nat Biotechnol. 2022 February; 40(2):227-234. doi: 10.1038/s41587-021-01026-y. Epub 2021 Oct. 14. PMID: 34650270; PMCID: PMC8847310. The prime editor may also constitute a fusion protein or separate proteins comprising a Cas9 nickase (nCas9), a reverse transcriptase (RT) and a third enzyme (such as serine recombinase Bxb1, TP901 or phiBT1) that function together to append a sequence to the genome or alter the local genomic sequence (change, insert, delete or insert and delete DNA sequence). Andrew V. Anzalone, Xin D. Gao, Christopher J. Podracky, Andrew T. Nelson, Luke W. Koblan, Aditya Raguram, Jonathan M. Levy, Jaron A. M. Mercer & David R. Liu, Nat Biotechnol. 2021. PMID: 34887556. DOI: 10.1038/s41587-021-01133-w. In principle, a wide variety of reverse transcriptases and Cas9 orthologs and PE-based variants can function in the context of prime editors.

As used herein, the term “pegRNA” refers to a RNA sequence composed of an sgRNA compatible with the Cas9 protein of a prime editor and a 3′ extension that encodes sequences to template the incorporation of a defined sequence into the genome by the reverse transcriptase of the prime editor. The 3′ extension comprises at least two different elements: the primer binding site (PBS) and the reverse transcriptase template (RT template or RTT). The primer binding site sequence is complementary to the 3′ end of the nicked non-template strand. After formation of the R-loop complex by the prime editor and nicking of the non-template strand, this genomic sequence can anneal to the PBS region of the pegRNA. The 3′ end of the genome can then serve as a primer that is extended by the reverse transcriptase to add the sequence (polymerize via DNA synthesis) that is present within the reverse transcriptase template to the 3′ end of the genomic sequence. In vitro this tail or flap is added to only a single strand of the genome. In cells this tail can be recognized by DNA repair enzymes and incorporated into the genome based on the present of a homology arm (HA) encoded at the 5′ end of the RT template region of the pegRNA. Anzalone, A. V. et al. Search-and-replace genome editing without double-strand breaks or donor DNA. Nature 576, 149-157 (2019). The term “pegRNA” may also refers to a separate sgRNA and RTT-PBS template (delivered as RNA or DNA), where the latter is used as an in trans template for off-target analysis.

The term “cognate” as used herein, refers to the complementarity status between a pegRNA spacer sequence and a target sequence. Similarly this term can reflect the complementarity status between the nCas9 protein and the PAM within any potential recognition sequence.

The term “fully-cognate” as used herein, refers to a completely identical (e.g., 100%) complementarity status between a pegRNA spacer sequence and a target sequence. For example, the pegRNA spacer sequence and target sequence are perfectly complementary. Similarly this term can reflect perfect complementarity between the nCas9 protein and the PAM within any potential recognition sequence.

The term “near-cognate” as used herein, refers to a partially identical (e.g., <100%) complementarity status between a pegRNA spacer sequence and a target sequence. For example, the pegRNA spacer sequence and target sequence are partially complementary. Similarly this term can reflect the partial complementarity between the nCas9 protein and the PAM within any potential recognition sequence.

As used herein, the term “prime editor recognition sequence” refers to a DNA sequence that can be hybridized to, and modified by, a prime editor protein/-pegRNA complex. The pegRNA comprises an encoded spacer sequence that is complementary to the DNA sequence and a compatible PAM of a primer editor nCas9 protein. A prime editor recognition sequence (or functional sequence) can be the intended target site within a genome that is perfectly complementary (fully-cognate) to a pegRNA spacer sequence and/or a compatible PAM for the prime editor nCas9 protein (e.g., a functional on-target sequence). In addition, a prime editor recognition sequence (or functional sequence) can be the intended target site within a genome that is partially complementary (near-cognate) to a pegRNA spacer sequence and/or a compatible PAM for a prime editor Cas9 protein (e.g., a functional off-target sequence). Typically prime editing at functional off-target sequences will be less efficient than editing at the target site. See, Anzalone, A. V. et al. Search-and-replace genome editing without double-strand breaks or donor DNA. Nature 576, 149-157 (2019). Kim, D. Y., Moon, S. B., Ko, J.-H., Kim, Y.-S. & Kim, D. Unbiased investigation of specificities of prime editing systems in human cells. Nucleic Acids Res 48, 10576-10589 (2020).

As used herein, the term “reverse transcriptase” refers to an enzyme used to generate complementary DNA (cDNA) from an RNA template in a process termed reverse transcription. Reverse transcriptases (RTs) are encoded by a variety of viruses, such as murine leukemia virus (MMLV) and HIV-1, and selfish genetic elements. Using a primer that is annealed to an RNA template RTs can polymerize DNA from deoxyribonucleoside triphosphates that is complementary to the RNA sequence. Some reverse transcriptases can also employ DNA as a template for primer-dependent copying of a complementary strand. In principle a wide variety of reverse transcriptases or DNA polymerases (when RTT-PBS template delivered as a DNA) can function in the context of prime editors to use the 3′ nicked genome annealed to the PBS element to polymerize DNA complementary to the RTT to do 3′ extension.

As used herein, the term “off-target site” (OT) refers to an unintended sequence within the genome where the prime editor is also functional. The target site is defined by the spacer sequence within the pegRNA and the PAM specificity of the Cas9 nickase that is present in the prime editor. An off-target site is usually a near cognate sequence to the target site that can also be recognized by the prime editor where some level of enzymatic activity is present, but not necessarily as high as the target site. DNA nicking at the off-target site by the Cas9 nickase can result in mutations (e.g., insertions or deletions; indels). In addition, the reverse transcriptase may be functional at the off-target site leading to template directed sequence alterations if the 3′ end of the nicked DNA has sufficient complementarity to the PBS sequence within the pegRNA. Off-target editing within the genome is undesirable, as it may have unintended negative consequences. For example, prime editing at the OT may in the context of editing at the target site or another OT site result in large-scale genomic rearrangements (translocations, deletions and inversions) in the genome, especially when a nicking sgRNA or two pegRNA are used.

As used herein, the term “target site” or “target sequence” refers to a sequence that can be recognized by the prime editor based on the pegRNA present within the complex and the PAM preference of the encoded nCas9 component. Typically, the target site will be fully complementary with the spacer sequence encoded within pegRNA complexed with the prime editor and will have a cognate PAM for the nCas9 component. However, a target sequence can also be a near-cognate sequence to the spacer sequence encoded within the pegRNA complexed with the prime editor and will have a cognate or near-cognate PAM for the nCas9 component. A genome can contain multiple target sequences for the prime editors based on the spacer sequence encoded within the pegRNA complexed with the prime editor and the PAM preference of the nCas9 component

As used herein, the term “on-target site” refers to a sequence that can be recognized by the prime editor based on the pegRNA present within the complex and the PAM preference of encoded nCas9 component. Typically, the target site will be fully complementary with the spacer sequence encoded within pegRNA complexed with the prime editor and will have a cognate PAM for the nCas9 component, as it will be the intended target for editing within the genome. However, it is possible to intentionally target a sequence when the spacer sequence encoded within the pegRNA complexed with the prime editor is near-cognate to the on-target site or a near-cognate PAM for the nCas9 component is present within the on-target site.

As used herein, the terms “validated on-target site” and “validated off-target site” means that gene editing at a particular on-target or off-target site has been confirmed. Usually, this confirmation is provided by deep sequencing to provide evidence that the nucleotide sequence of the target site has been changed.

As used herein, the term “amplification tag element” refers to a DNA sequence that is appended or introduced into a genome by a prime editor that permits the selective amplification of genomic regions with prime editor activity (e.g., a functional sequence or gene edited target sequence). The amplification tag element may be encoded within an RT template region of a pegRNA. For in vitro prime editor reactions this sequence is added to the 3′ end of the nicked genome at any genomic region where the prime editor is functional for nicking the DNA and where this genomic region can anneal to the PBS sequence of the pegRNA for a reverse transcriptase to extend the sequence. This amplification tag element addition can take place at a target site defined by a spacer sequence within the pegRNA and the PAM specificity of the Cas9 nickase that is present in the prime editor. This amplification tag element addition can also take place at the other near-cognate sequences to the target site within the genome (e.g. off-target sites). These near-cognate sequences where the amplification tag element is added represent potential off-target sequences within the genome where gene editing activity by a prime editor may be undesirable. The addition of the amplification tag element within the genome at all active prime editor sites allows these genomic regions to be amplified selectively following Tn5 tagmentation to introduce universal adaptors throughout the genome. Once genomic regions with the amplification tag have been amplified by PCR, they can be sequenced by Illumina deep sequencing to identify these genomic regions. The term “tag” is understood in the field as a sequence element that when added to the genome labels it for selective amplification. For example, see FIG. 1 from Tsai, S. Q. et al. GUIDE-seq enables genome-wide profiling of off-target cleavage by CRISPR-Cas nucleases. Nat Biotechnol 33, 187-197 (2015).

As used herein, the term “Tn5 tagmentation” or “tagmenting with a Tn5 transposase enzyme” refers to the process of breaking the genome into short fragments that have universal adaptor sequences appended to the genome. Tn5 transposase is loaded with duplex oligonucleotides that contain a Tn5 recognition sequence and a universal adaptor sequence. The Tn5 complex then breaks the genome into DNA fragments in the process of inserting a universal adaptor sequence.

As used herein, the term “universal adaptor” contains a sequence for selective amplification of genomic fragments and may also contain other elements such as a unique molecular identifier (UMI) and a barcode for allowing multiple experiments to be pooled in a single deep sequencing run. For example see FIG. 1A. Giannoukos, G. et al. UDiTaS™, a genome editing detection method for indels and genome rearrangements. BMC genomics 19, 212 (2018).

As used herein, the term “unique molecular identifier” or “UMI” sequence refers to a population of randomized sequences that allow unique Tn5 tagmentation events to be counted based on the sequence encoded within this element. The UMI can be used to avoid amplification bias—there can be many sequencing reads for a specific UMI sequence associated with a given genomic locus due to the exponential amplification that occurs during PCR. The UMI sequence allows all sequencing reads initiating at a given locus to that share an identical UMI sequence to be collapsed down to a single sequence read to count unique editing events within the genome.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10(11): 1116-1121 (2013). For example, this would allow one Cas9 isoform (e.g. S. pyogenes Cas9 or SpyCas9) to function as a nuclease programmed by a sgRNA that may be specific to it, and another Cas9 isoform (e.g. N. meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March; 32(3):279-284 (2014).

The term “base pairs” as used herein, refer to specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic segment, the recoding of the DNA sequence (e.g. encoding base transitions or transversion), the specific inclusion of new sequence through the use of an exogenously supplied DNA or RNA template, or the deletion and insertion of new sequences through the use of an exogenously supplied DNA or RNA template. Such a specific genomic target includes, but may be not limited to, a chromosomal region, mitochondrial DNA, a gene, a promoter, an open reading frame or any nucleic acid sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” or “linked to” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that all trace impurities have been removed.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

The terms “complementary” or “complementarity”, as used herein, are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

Low stringency conditions comprise conditions equivalent to binding or hybridization at 42° C. in a solution consisting of 5×SSPE (43.8 g/l NaCl, 6.9 g/l NaH₂PO₄·H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ tor R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term, “deoxyribonucleic acid” or “DNA” as used herein, refer to molecules having “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

### BRIEF DESCRIPTION OF THE FIGURES

FIG. 1 presents exemplary data showing genome-wide detection of prime editing by PE-tag in vitro in the human genome. Mismatches relative to the target site (On) are shown with colored boxes. UMI numbers for each site are shown (average of three independent experiments).

FIG. 1A: Schematic overview of in vitro PE-tag. gDNA of interest is isolated from cells and treated with PE2 protein and a 20-7 tagging pegRNA with a spacer sequence complementary to the desired target site. Prime editing results in a 3′ flap containing a 20 bp tag within the protospacer of the target site and any active off-target sites. Tn5 enzyme tagments genomic DNA and adds UMI, a pooling barcode, and i5 primer sites. PE active genomic regions are amplified with i5 Primer and a tag-specific reverse primer (Tag primer_R). A second round of PCR adds the i7 adaptor sequence. Deep sequencing captures the PAM distal sequence.

FIG. 1B: An IGV image of read coverage for PE-tag insertion of a reverse primer (tag primer_R) that reveals the non-target strand at the HEK4 site. Top trace: Raw reads mapped to the locus. Bottom trace: Strand-specific reads.

FIG. 1C: potential off-target (OT) sites identified by in vitro PE-tag at the HEK4 locus with human genome treated with PE2 RNP. Mismatches in the PBS and HA region of the RTT relative to the target site (On) are shown in red and blue, respectively. UMI numbers for each site are shown. GUIDE-seq and nDigenome-seq read counts for each OT site have been previously described. Tsai, S. Q. et al. GUIDE-seq enables genome-wide profiling of off-target cleavage by CRISPR-Cas nucleases. Nat Biotechnol 33, 187-197 (2015). Kim, D. Y., Moon, S. B., Ko, J. H., Kim, Y. S. & Kim, D. Unbiased investigation of specificities of prime editing systems in human cells. Nucleic Acids Res 48, 10576-10589 (2020).

FIG. 1D: (left) Venn diagram of overlap between potential off-target sites discovered by (top) in vitro PE-tag (UMI>1) and GUIDE-seq or for (bottom) in vitro PE-tag (UMI>1) and nDigGenome-seq at HEK4 site. (right) The overlap between the top 10 potential off-target sites for in vitro PE-tag and the potential off-target sites discovered by (top) GUIDE-seq or (bottom) nDigGenome-seq.

FIG. 1E: Percentages of validated OTs (n=3) from in vitro PE-tag and GUIDE-seq by targeted amplicon deep sequencing after delivering PE2 and pegRNA expression plasmids encoding different HEK4 mutations to HEK293T cells.

FIG. 2 presents exemplary electrophoretic data showing that Cas9 H₈₄₀A and MMLV RT proteins are functional independently in vitro in a PE-tag system.

FIG. 2A: Schematic overview of the in vitro tag attachment in the human genome by purified PE2 or purified Cas9 H840A nickase and MMLV RT. gDNA is isolated from HEK293T cells and treated with indicated protein and a 20-7 pegRNA, resulting in a 20 bp tag attachment in the protospacer of on-target site.

FIG. 2B: A locus specific primer was used in conjunction with a tag-specific primer to amplify each locus by PCR if the tag element had been added into the human genome at the target site (on-target) and off-target site 3 (OT-3). PE-tag was carried out in vitro on purified human genomic DNA with three different protein cocktails: 1) purified PE2 protein; 2) separately purified Cas9 H840A nickase and MMLV RT proteins; and 3) Cas9 H840A nickase (IDT) and MMLV RT (Thermofisher) using the HEK4 targeting pegRNA.

FIG. 2C: Venn diagram of overlap of PE potential off-target sites (UMI≥1) discovered by three different protein cocktails using in vitro PE-tag.

FIG. 2D: In vitro off-target (OT) sites identified. Mismatches in the PBS and HA region of potential off-target sites relative to the target site (On) are shown in red and blue, respectively. UMI counts for each site are shown.

FIG. 3 presents exemplary data showing PE-tag amplification tag element integration at on-target site and off-target sites in human cells. All expression vectors were delivered by transient transfection. Results were obtained from three independent experiments and presented as mean±SD.

FIG. 3A: Schematic of pegRNAs expression cassette used for amplification tag element insertion includes a spacer sequence, a sgRNA scaffold, a RT template including different amplification tag elements (7 bp, 11 bp and 20 bp) and different lengths of homology arms (7 bp, 16 bp and 20 bp) within the RT template (RTT) and a primer binding site (PBS).

FIG. 3B: Sequence of HEK 4 locus (On) and two off-target sites (OT-1 and OT-3) after amplification tag element insertion with different pegRNAs. Mismatches are labeled in red.

FIG. 3C: Comparison of editing efficiency for different amplification tag elements inserted with PE2 at HEK4 target site and two OT sites in HEK293T cells. Editing rates are determined by Illumina sequencing PCR amplicons spanning each locus.

FIG. 4 presents exemplary deep sequencing frequency data showing PE-tag integration of amplification tag elements at the target site and an off-target site in human cells. Prime editing components were delivered as expression plasmids by transient transfection to HEK293T cells.

FIG. 4A: Editing efficiency with PE2 at HEK4 site 4 locus (On) in HEK293T cells.

FIG. 4B: Editing efficiency with PE2 at OT3 in HEK293T cells.

FIG. 5 presents exemplary data showing in vitro PE-tag in human genome by PE2 RNP.

FIG. 5A: Coomassie Blue stained SDS-PAGE gel of Ni-NTA purified C-terminally His-tagged PE2 protein.

FIG. 5B: Schematic overview of in vitro PE-tag attachment of an amplification tag element at active prime editing sites in purified human genomic DNA (gDNA). gDNA is isolated from cells and treated with PE2 protein and a pegRNA 20-7, resulting in a 20 bp amplification tag element insertion in the protospacer of target and potential off-target sites.

FIG. 5C: Junction PCRs with locus-specific primers and tag primer R were performed on gDNA treated with 20-7 PE2 RNP targeting the HEK4 locus to detect the amplification tag element insertion.

FIG. 6 presents exemplary data showing in vitro PE-tag in the human genome.

FIG. 6A: Potential off-target (OT) sites identified by in vitro PE-tag in PE2 RNP-treated human gDNA at CDH4 locus. Mismatches in the PBS and HA region of potential off-target sites relative to the target site (On) are shown in red and blue, respectively. UMI numbers for each site are shown (average of three independent experiments).

FIG. 6B: Potential off-target (OT) sites identified by in vitro PE-tag in PE2 RNP-treated human gDNA at VEGFA locus. Mismatches in the PBS and HA region of potential off-target sites relative to the target site (On) are shown in red and blue, respectively. UMI numbers for each site are shown (average of three independent experiments).

FIG. 6C: Venn diagram of overlap between off-target sites discovered by in vitro PE-tag (UMI>1) and previously described GUIDE-seq data for VEGFA site 2. Tsai, S. Q. et al. GUIDE-seq enables genome-wide profiling of off-target cleavage by CRISPR-Cas nucleases. Nat Biotechnol 33, 187-197 (2015).

FIG. 6D: Chromosome diagram of PE2-tag on-target sites (green) and potential off-target sites (red: UMI>50; blue: 5<UMI<50) determined for PE2 targeting HEK4.

FIG. 7 presents exemplary data showing prime editing at on-target sites and off-target sites in human cells. Frequencies of precise editing were quantified by deep sequencing from PCR amplicons spanning each locus. Results were obtained from three independent experiments and presented as mean±SD.

FIG. 7A: (left) Comparison of precise editing efficiency for nucleotide substitution, targeted 1-bp deletion, and 1-bp insertion with PE2 at HEK4 (ON) target site and (right) indel rates at two off-target sites (OT-1 and OT-3) in HEK293T cells after co-transfecting pegRNA and PE2 expression plasmids. pegRNA sequence composition and type of sequence modification encoded is indicated in the legend, where the terminal numbers indicate the different HA lengths within the RTT. Frequencies of precise editing or indel rates were quantified by deep sequencing from PCR amplicons spanning each locus. Mock on target site editing represents all indels. Results were obtained from three independent experiments and presented as mean±SD. *P<0.05, **P<0.01 and ***P<0.001 by unpaired, two-tailed Student's t-test. To adjust for multiple comparisons, p-values were adjusted using the Benjamini-Hochberg (BH) method.

FIG. 7B: Indel rates for nucleotide substitution, targeted 1-bp deletion, and 1-bp insertion pegRNAs with PE2 at 6 additional potential off-target sites identified by PE-tag for HEK4 locus pegRNA in HEK293T cells. pegRNA sequence composition and type of sequence modification encoded is indicated in the legend, where the terminal numbers indicate the different HA lengths within the RTT. Frequencies of precise editing were quantified by deep sequencing from PCR amplicons spanning each locus. Results were obtained from three independent experiments and presented as mean±SD. *P<0.05, **P<0.01 and ***P<0.001 by unpaired, two-tailed Student's t-test. To adjust for multiple comparisons, p-values were adjusted using the Benjamini-Hochberg (BH) method.

FIG. 8 presents exemplary data showing genome-wide detection of prime editing by PE-tag in cells.

FIG. 8A: Schematic overview of the PE-tag method in cells. PE2 RNP or expression plasmids are delivered to cells with the 20-7 tagging pegRNA, resulting in a 20 bp amplification tag element insertion at the target site and active off-target sites. Tn5 tagmentation of the purified genomic DNA from these cells adds unique molecular identifier (UMI), pooling barcode, and i5 primer sites. Genomic regions with active prime editing are amplified with i5 primer and one of two tag-specific primers (Tag primer_F or Tag primer_R) in separate library builds that hybridize to the integrated amplification tag element. A second round of PCR adds the i7 adaptor sequence. Deep sequencing captures 5′ and 3′ flanking sequences around the tag insertion sites.

FIG. 8B: An IGV image of read coverage for forward (F) primer and reverse (R) primer reveals sequencing reads originating within the non-target and target strands, respectively, at the HEK4 locus. Top trace: Raw reads mapped to the locus. Bottom trace: Strand-specific reads.

FIG. 8C: Potential off-target (OT) sites identified by PE-tag in PE2 RNP or plasmid treated cells at HEK4 locus. Mismatches in the PBS and HA region of the RTT for OT sites relative to the target site (On) are shown in red and blue, respectively. UMI counts for each site are shown (average of three independent experiments).

FIG. 8D: Venn diagram of overlap of PE potential off-target sites discovered by in vitro PE-tag, PE-tag in cells using expression plasmids and PE-tag in cells using RNPs at HEK4 site and VEGFA site.

FIG. 9 presents exemplary data showing PE-tag in cells.

FIG. 9A: Potential off-target (OT) sites identified by PE-tag using PE2 RNP or expression plasmid treated cells at the VEGFA locus. Mismatches in the PBS and HA region for potential off-target sites relative to the target site (On) are shown in red and blue, respectively. UMI counts for each site are shown for each treatment.

FIG. 9B: Potential off-target (OT) sites identified by PE-tag using PE2 RNP or expression plasmid treated cells at the CDH4 locus. Mismatches in the PBS and HA region for potential off-target sites relative to the target site (On) are shown in red and blue, respectively. UMI counts for each site are shown for each treatment

FIG. 9C: potential off-target sites (UMI>1) discovered by in vitro PE-tag in HEK293T cells and U2OS cells for pegRNA and epegRNA at HEK4 site. HEK293T cells and U2OS cells were electroporated with plasmids expressing PE2 and 20-7 tagging pegRNA or epegRNA.

FIG. 9D: Bar chart shows the number of potential off-target sites falling into different groups based on a range of recovered UMI counts by in vitro PE-tag in HEK293T cells and U2OS cells for pegRNA and epegRNA at HEK4 site.

FIG. 9E: Tag insertion efficiency for 20-bp amplification tag element insertion by PE2 at HEK4 site in HEK293T cells and U2OS cells. Frequencies of precise editing were quantified by deep sequencing from PCR amplicons spanning the HEK4 locus.

FIG. 10 presents exemplary data showing in vivo PE-tag at the Pcsk9 locus in mouse primary liver cells.

FIG. 10A: Schematic of components for evaluating PE-tag in the mouse liver. B6 mice were injected by hydrodynamic tail vein injection (HTVI) with a PE2 expression plasmid and a pegRNA to insert a 20 bp amplification tag element at the Pcsk9 locus.

FIG. 10B: Identification of potential off-target (OT) sites by PE-tag at Pcsk9 locus. gDNA from B6 mice was used for in vitro PE-tag and isolated for in vivo PE-tag following HTVI treatment. PE-tag in cells used a Hepa1-6 cell line electroporated with PE2 RNP or plasmids expressing PE2 and a pegRNA/epegRNA for the 20 bp tag insertion at Pcsk9 locus. Mismatches in the PBS and HA region of the RTT at potential OT sites relative to the target site (On) are shown in red and blue, respectively. UMI counts for each site are shown.

FIG. 10C: Venn diagram of overlap of potential off-target sites (UMI>1) discovered by in vitro PE-tag, PE-tag in cells by plasmid delivery and PE-tag in cells by RNP delivery at Pcsk9 site.

FIG. 11 presents exemplary data showing editing efficiency of Pcsk9 off-target sites in Hepa1-6 cells. Indel frequencies were quantified by deep sequencing from PCR amplicons spanning each locus. Results were obtained from three independent experiments and presented as mean±SD. *P<0.05 and **P<0.01 by unpaired, two-tailed Student's t-test. To adjust for multiple comparisons, p-values were adjusted using the Benjamini-Hochberg (BH) method.

FIG. 12 presents exemplary data showing PE-tag identification of off-target editing associated with therapeutically relevant targets.

FIG. 12A: The known pathogenic human genetic variants in ClinVar, classified by type.

FIG. 12B: Three human pathogenic mutations (CFTRΔF508, HEXA1278+TATC, MeCP2T158M) used for PE-tag analysis. Cell lines containing indicated mutant sequences were electroporated with plasmids expressing PE2 and pegRNAs or epegRNAs for correction of indicated mutations, or PE2 mRNA and synthetic pegRNAs. Precise editing for each target site was quantified by amplicon deep sequencing. Results were obtained from three independent experiments and presented as mean±SD.

FIG. 12C: Potential off-target (OT) sites identified by PE-tag at the CFTR locus in a cell line harboring this mutation. Top five potential OT sites identified by in vitro PE-tag are shown. Mismatches in the PBS and HA region of the RTT relative to the target site (On) are shown in red and blue, respectively. UMI counts for each site are shown (average of three independent experiments). Potential OTs edited by SpCas9 RNP for each site were determined by GUIDE-tag. SpCas9 Nuclease activity for each site (indel %) was measured by amplicon deep sequencing.

FIG. 12D: Potential off-target (OT) sites identified by PE-tag at the HEXA locus a cell line harboring this mutation. Top five potential OT sites identified by in vitro PE-tag are shown. Mismatches in the PBS and HA region of the RTT relative to the target site (On) are shown in red and blue, respectively. UMI counts for each site are shown (average of three independent experiments). Potential OTs edited by SpCas9 RNP for each site were determined by GUIDE-tag. SpCas9 Nuclease activity for each site (indel %) was measured by amplicon deep sequencing.

FIG. 12E: Potential off-target (OT) sites identified by PE-tag at the MECP2 locus cell line harboring this mutation. Top five potential OT sites identified by in vitro PE-tag are shown. Mismatches in the PBS and HA region of the RTT relative to the target site (On) are shown in red and blue, respectively. UMI counts for each site are shown (average of three independent experiments). Potential OTs edited by SpCas9 RNP for each site were determined by GUIDE-tag. SpCas9 Nuclease activity for each site (indel %) was measured by amplicon deep sequencing.

FIG. 12F: Editing efficiency for each OTs in cells transfected with PE2 mRNA, plasmids expressing PE2 and pegRNA or epegRNA designed to remove the four base insertion at the HEXA locus. *P<0.05, **P<0.01 and ***P<0.001 by unpaired, two-tailed Student's t-test. To adjust for multiple comparisons, p-values were adjusted using the Benjamini-Hochberg (BH) method.

FIG. 12G: Dot plot of percent UMI counts at each identified potential off-target site for 2 pegRNAs analyzed by in vitro PE-tag, PE-tag in cells by plasmid delivery and PE-tag in cells by RNP or mRNA delivery. Circles: in vitro PE-tag; Triangles: in vivo PE-tag by plasmid; and Squares: in vivo PE-tag by RNP or mRNA. Red: On-target sites. Black: Off-target sites. Blue/Green: most active OT.

FIG. 12H: Ratio of on target to off targets editing.

FIG. 13 presents exemplary data showing in vitro PE-tag in human genome at three pathogenic sites by PE2 RNP.

FIG. 13A: gDNA were isolated from three cell lines with sequences containing the indicated pathogenic mutations. The gDNA was treated with PE2 protein and a pegRNA 20-7 to add the amplification tag element. Junction PCR with a locus-specific primer and tag primer R were performed to detect amplification tag element insertion.

FIG. 13B: The amplification tag element attachment was validated Sanger sequencing.

FIG. 14 presents exemplary data showing sequencing reads generated subsequent to in vitro PE-tag and GUIDE-tag at three pathogenic on-target sites and OT-1 by the indicated primers.

FIG. 14A: IGV images of read coverage for in vitro hybridization of PE-tag inserted amplification tag elements to a reverse primer (tag primer R) and a GUIDE-tag forward primer (F) and reverse primer (R) that sequence out from each inserted tag into the non-target and target strands, respectively, at the CFTR locus. Top trace: Raw reads mapped to the locus. Bottom trace: Strand-specific reads.

FIG. 14B: IGV images of read coverage for in vitro hybridization of PE-tag inserted amplification tag elements to a reverse primer (tag primer R) and a GUIDE-tag forward primer (F) and reverse primer (R) that sequence out from each inserted tag into the non-target and target strands, respectively, at the HEXA locus. Top trace: Raw reads mapped to the locus. Bottom trace: Strand-specific reads.

FIG. 14C: IGV images of read coverage for in vitro hybridization of PE-tag inserted amplification tag elements to a reverse primer (tag primer R) and a GUIDE-tag forward primer (F) and reverse primer (R) that sequence out from each inserted tag into the non-target and target strands, respectively, at the MECP2 locus. Top trace: Raw reads mapped to the locus. Bottom trace: Strand-specific reads.

FIG. 15 presents exemplary data showing prime editing off-target editing rates at off-target sites for pegRNAs designed to correct three pathogenic mutations.

FIG. 15A: Venn diagram of overlap between potential off-target sites (UMI>1) discovered by in vitro PE-tag and potential off-target sites discovered by GUIDE-tag in cell lines containing the pathogenic sequences treated with SpCas9 RNP and DSB tagging oligonucleotide.

FIG. 15B: Comparison of editing rates by prime editor programmed with pegRNA to correct pathogenic sequence at each potential OT site in cells transfected with PE2 mRNA and pegRNA, plasmids expressing PE2 and pegRNA, or plasmids expressing PE2 and epegRNA at the CFTR locus. Frequencies of editing rates were quantified by deep sequencing. Results were obtained from three independent experiments and presented as mean±SD. *P<0.05, **P<0.01 and ***P<0.001 by unpaired, two-tailed Student's t-test. To adjust for multiple comparisons, p-values were adjusted using the Benjamini-Hochberg (BH) method.

FIG. 15C: Comparison of editing rates by prime editor programmed with pegRNA to correct pathogenic sequence at each potential OT site in cells transfected with PE2 mRNA and pegRNA, plasmids expressing PE2 and pegRNA, or plasmids expressing PE2 and epegRNA at the MECP2 locus. Frequencies of editing rates were quantified by deep sequencing. Results were obtained from three independent experiments and presented as mean±SD. *P<0.05, **P<0.01 and ***P<0.001 by unpaired, two-tailed Student's t-test. To adjust for multiple comparisons, p-values were adjusted using the Benjamini-Hochberg (BH) method.

FIG. 16 presents an illustrative schematic of prime editor protein and pegRNA engaged with the target site following nicking of the non-target strand by Cas9 H840A nickase (nCas9). The complementary region of the nicked non-target strand anneals to the primer binding site (PBS) within the pegRNA where it can be extended by the fused MMLV reverse transcriptase (RT) with the incorporated nucleotides defined by the sequence of the reverse transcriptase template (RTT) region of the pegRNA.

FIG. 17 presents exemplary data showing a dot plot of UMI count percentage (UMI %) associated with the target site and discovered potential off-target sites for 5 pegRNAs analyzed by in vitro PE-tag, PE-tag in cells by PE2 plasmid delivery and PE-tag in cells by PE2 RNP or mRNA delivery. Red: On-target sites. Black: Off-target sites.

FIG. 18 presents a schematic overview of PE-tag in vitro and PE-tag in vivo or in cells providing a comparison of the approaches.

FIG. 19 presents exemplary data of PE-Tag 3′-flap introduction.

FIG. 19A: A schematic overview of quantification of 3′ flap generation by qRT-PCR at the HEK4 locus. HEK293T gDNA was treated with PE2 RNP to introduce the 3′ flap and then the editing efficiency was quantified by qRT-PCR with a tag-specific primer and a locus-specific primer. A pair of primers located ~2000 bp upstream of the target site serve as an internal control for gDNA normalization.

FIG. 19B: HEK293T gDNA was treated with different concentrations of PE2 RNP to introduce the 3′ flap and then the editing efficiency was quantified by qRT-PCR.

FIG. 19C: HEK293T gDNA was treated with 50 μmol of PE2 RNP to introduce the 3′ flap for different reaction times and then the editing efficiency was quantified by qRT-PCR. Results were obtained from three independent experiments and presented as mean±SD. ****P<0.0001 by one-way ANOVA with Tukey's multiple comparisons test.

FIG. 19D: HEK293T gDNA was treated with 50 μmol of PE2 RNP to introduce the 3′ flap in a buffer containing different concentrations of dNTPs and then the editing efficiency was quantified by qRT-PCR. Results were obtained from three independent experiments and presented as mean±SD. ****P<0.0001 by one-way ANOVA with Tukey's multiple comparisons test.

FIG. 19E: HEK293T gDNA was treated with 50 μmol of PE2 RNP to introduce the 3′ flap at different reaction temperatures and then the editing efficiency was quantified by qRT-PCR. Results were obtained from three independent experiments and presented as mean±SD. **P<0.01 and ***P<0.001 by one-way ANOVA with Tukey's multiple comparisons test.

FIG. 19F: HEK293T gDNA was treated with 50 μmol of PE2 RNP to introduce the 3′ flap for two different reaction times (2 hrs and 24 hrs) and then the editing efficiency was quantified by qRT-PCR on target site and two OTs for the HEK4 site. Results were obtained from three independent experiments and presented as mean±SD. ****P<0.001 by unpaired, two-tailed Student's t-test.

FIG. 20 presents exemplary data showing gene editing efficiency with PBS mismatches.

FIG. 20A: The prime editing efficiency of 3′ flap generation with a series of pegRNAs which contain either one or two mismatches in the PBS region. HEK293T gDNA was treated with PE2 RNP containing the HEK4 20-7 pegRNA to introduce the 3′ flap for 2 hours, and then the flap incorporation efficiency was quantified by qRT-PCR with a tag-specific primer and a locus-specific primer. A pair of primers located ~2000 bp upstream of the target site serve as an internal control for data analysis.

FIG. 20B: The efficiency of 3′ flap generation at HEK OT3 with a series of HEK4 20-7 pegRNAs which contain either one or two mismatches in the PBS region. HEK293T gDNA was treated with PE2 RNP to introduce the 3′ flap, and then the editing efficiency was quantified by qRT-PCR with a tag-specific primer and a locus-specific primer. A pair of primers located ~2000 bp upstream of the target site serve as an internal control for data analysis. Where shown, bar charts indicate the mean and error bars are s.d. of n=3 independent qRT-PCR experiments.

FIG. 21 presents a subset of potential off-target (OT) sites identified by in vitro PE-tag in PE2 RNP treated HEK293T gDNA at VEGFA locus.

FIG. 21A: Mismatches in the PBS and HA region of potential off-target sites relative to the target site (On) are shown in red and blue, respectively. UMI counts for each site are shown.

FIG. 21B: Venn diagram of overlap between off-target sites discovered by in vitro PE-tag (UMI>1) and previously described GUIDE-seq data for VEGFA site 211.

FIG. 22 presents exemplary data showing indel rates of nucleotide substitutions at off-target sites.

FIG. 22A: Indel rates for nucleotide substitution, targeted 1-bp deletion, and 1-bp insertion pegRNAs with PE2 at 8 potential off-target sites of top 20 OTs identified by GUIDE-seq that overlap with in vitro PE-tag for HEK4 locus pegRNA in HEK293T cells. Frequencies of editing were quantified by deep sequencing from PCR amplicons spanning each locus. Results were obtained from three independent experiments and presented as mean±SD. *P<0.05, **P<0.01 and ***P<0.001 by two-way ANOVA with Tukey's multiple comparisons test.

FIG. 22B: Indel rates for nucleotide substitution, targeted 1-bp deletion, and 1-bp insertion pegRNAs with PE2 at 12 potential off-target sites of top 20 OTs identified by GUIDE-seq but absent in the in vitro PE-tag for HEK4 locus pegRNA in HEK293T cells. pegRNA sequence composition and type of sequence modification encoded is indicated in the legend, where the terminal numbers indicate the different HA lengths within the RTT. Frequencies of editing were quantified by deep sequencing from PCR amplicons spanning each locus. Results were obtained from three independent experiments and presented as mean±SD. *P<0.05, **P<0.01 and **P<0.001 by two-way ANOVA with Tukey's multiple comparisons test.

FIG. 22C: Editing outcomes with PE2 and 1-bp deletion pegRNA at HEK4 MISS-2 and MISS-7 in HEK293T cells. Frequencies of editing were quantified by deep sequencing of PCR amplicons spanning the locus. CRISPResso output shown for sequencing data.

FIG. 23 presents exemplary data showing indel rates of tag insertions at off-target sites.

FIG. 23A/B: Identification of genomic locations of prime editing in cells.

FIG. 23C: Indel rates for tag insertion pegRNAs with PE2 at top 5 potential off-target sites identified by in vitro PE-tag for VEGFA locus pegRNA in HEK293T cells. Indel frequencies were quantified by deep sequencing from PCR amplicons spanning each locus. Results were obtained from three independent experiments and presented as mean±SD. **P<0.01 and ***P<0.001 by unpaired, two-tailed Student's t-test. To adjust for multiple comparisons, p-values were adjusted using the Benjamini-Hochberg (BH) method.

FIG. 24 presents exemplary data of off target site validation in HEK293T cells.

FIG. 24A: Editing efficiency with PE2 at HEK4 OT-10 in HEK293T cells. Frequencies of editing were quantified by deep sequencing of PCR amplicons spanning the locus. CRISPResso output shown for sequencing data. Expected tag sequence is boxed in red and the homology arm is boxed in blue. RTT is boxed in black.

FIG. 24B: Editing outcomes at HEK4 OT-10 with PE2 complexed with different pegRNAs. For HEK4 peg20-7, most editing events are tag/RTT insertion with a modest number of genomic deletions. For HEK4 pegRNA encoding the G-T base substitution, 1-bp deletion, and 1-bp insertion, most editing events at OT-10 are genomic deletions with a small number of RTT region insertions. A frequently occurring 22 bp deletion (0.46% in G-T-7 pegRNA) in many of the treatment groups may be driven by local microhomologies (GCGGCTGGAG repeats). Results were obtained from three independent experiments and representative results are shown.

FIG. 25 presents a representative analysis of editing outcomes at three different target sites and related off-target sites.

FIG. 25A: The relative frequency of indels and tag insertion on 3 different target sites (HEK4, VEGFA and Pcsk9) and related highly active off-target sites with different pegRNAs by targeted amplicon deep sequencing. Results were obtained from three independent experiments and representative results are shown.

FIGS. 25B-C: Editing outcomes with PE2 and different pegRNAs at VEGFA in HEK293T cells (B) and Pcsk9 in Hepa1-6 cells (C). Frequencies of editing were quantified by deep sequencing of PCR amplicons spanning each locus. CRISPResso output shown for sequencing data.

FIG. 25D: The relative frequency of full length tag insertion and partial length tag insertion at 3 different target sites (HEK4, VEGFA and Pcsk9) and highly active related off-target sites with different pegRNAs by targeted amplicon deep sequencing. Results were obtained from three independent experiments and representative results are shown.

FIG. 26 presents a representative analysis of editing outcomes at HEK4.

FIG. 26A: Editing efficiency for nucleotide substitution, and targeted 1-bp deletion with PE2 or Cas9 H₈₄₀A nickase for HEK 4 locus pegRNA in HEK293T cells. Results were obtained from three independent experiments and presented as mean±SD.

FIG. 26B: Editing outcomes with PE2 and different pegRNAs at HEK4 in HEK293T cells. Frequencies of editing were quantified by deep sequencing of PCR amplicons spanning each locus. CRISPResso output shown for sequencing data.

FIG. 27 presents exemplary data comparing PE-tag in HEK293T and U2OS cells.

FIGS. 27A-B: A subset of potential off-target sites (UMI>1) discovered by PE-tag in both HEK293T cells and U2OS cells or only in HEK293T cells for pegRNA and epegRNA at HEK4 site. HEK293T cells and U2OS cells were electroporated with plasmids expressing PE2 and 20-7 tagging pegRNA or epegRNA. Bar chart shows the number of potential off-target sites falling into different groups based on a range of recovered UMI counts.

FIG. 27C: Tag insertion efficiency for 20-bp tag by PE2 at HEK4 site in HEK293T cells and U2OS cells. Frequencies of precise editing were quantified by deep sequencing from PCR amplicons spanning the HEK4 locus. Results were obtained from three independent experiments and representative results are shown.

FIG. 28 presents exemplary PE-tag data with PEmax.

FIG. 28A: A Venn diagram of overlap of potential off-target sites (UMI>1) discovered by in vitro PE-tag (RNP) and PE-tag in cells (plasmid) using expression plasmids at HEK4 site for PE2 and PEmax.

FIG. 28B: A subset of the potential off-target (OT) sites identified by PE-tag for PE2 or PEmax at HEK4 (Supplementary Data 1). Mismatches in the PBS and HA region of the RTT for OT sites relative to the target site (On) are shown in red and blue, respectively. UMI counts for each site are shown.

FIG. 29 illustrates a PE with a representative amplification tag.

FIG. 29B: Editing efficiency with PE2 and pegRNA containing an amplification tag insertion at HEK4 in HEK293T cells. Frequencies of editing were quantified by deep sequencing of PCR amplicons spanning the locus. CRISPResso output shown for sequencing data.

FIG. 29B: An IGV image of read coverage for PE-tag in cells with an amplification tag sequence with a library constructed using both forward and reverse primers (Tag primer_F and R) that reveals sequencing reads originating within the non-target and target strands, respectively, at the HEK4 site. Top trace in the plot is raw reads mapped to the locus. Bottom trace is strand-specific reads.

FIG. 29C: A Venn diagram of overlap of potential off-target sites (UMI>1) discovered by in vitro PE-tag and PE-tag in cells comparing two different amplification tags at the HEK4 site.

FIG. 30 presents exemplary data showing prime editing outcomes in cells at on target and off-target sites. Indel rates for nucleotide substitution, targeted 1-bp deletion, and 1-bp insertion with PE2 at six (6) potential off-target sites identified by in vitro PE-tag but not PE-tag in cells for the HEK4 locus pegRNA in HEK293T cells. pegRNA sequence composition and type of sequence modification encoded is indicated in the legend, where the terminal numbers indicate the different HA lengths within the RTT. Results were obtained from three independent experiments and presented as mean±SD. P-values are calculated by unpaired, two-tailed Student's t-test and none of the p-values are significant.

FIG. 31 presents exemplary data of prime editing at a subset of off-target sites for three pathogenic correcting pegRNAs.

FIG. 31A: A CRISPResso analysis of editing outcomes with PE2 and the therapeutic pegRNAs or SpCas9 with a corresponding sgRNA and OTs when delivered by expression plasmids at a CFTR site.

FIG. 31B: A CRISPResso analysis of editing outcomes with PE2 and the therapeutic pegRNAs or SpCas9 with a corresponding sgRNA and OTs when delivered by expression plasmids at a HEXA site.

FIG. 31C: A CRISPResso analysis of editing outcomes with PE2 and the therapeutic pegRNAs or SpCas9 with a corresponding sgRNA and OTs when delivered by expression plasmids at a MECP2 site.

Arrows indicate sequences from PE2 editing that contain the desired modification at the target site. Frequencies of precise editing were quantified by deep sequencing of PCR amplicons spanning the locus.

### DETAILED DESCRIPTION OF THE INVENTION

The present invention is related to the field of gene and cell therapy. In particular, to the clinical application of CRISPR-mediated gene and cell therapy. Herein, compositions and methods providing the ability to identify and quantitate on-target and off-target prime editor recognition sites are disclosed. These compositions include prime editors working in tandem with pegRNAs associated with an amplification tag element that is inserted at a genomic prime editor recognition site. The method contemplates a polymerase chain reaction amplification of these genomic target sites that are then subjected to sequencing to determine the location within the genome of functional prime editor recognition sites (on-target and off-target sites) and the relative activity at each of these target sites based on the number of distinct UMI sequences that are present at each locus.

Direct or indirect double strand break (DSB) detection is central to the existing nuclease-based genome-wide off-target analysis methods. Consequently, efforts to determine genome-wide off-target sites for prime editors systems have focused on either: 1) computational predictions of near-cognate sequences to the spacer sequence that is encoded by the pegRNA or 2) genome-wide off-target analysis for a nuclease or nickase programmed with an sgRNA recognizing the same target site as the pegRNA. Anzalone, A. V. et al. Nature 576, 149-157 (2019); Chen, P. J. et al. Cell 184, 5635-5652.e29 (2021); Nelson, J. W. et al. Nat Biotechnol 1-9 (2021); and Kim, D. Y., et. al. Nucleic Acids Res 48, 10576-10589 (2020).

Since these methods are not utilizing prime editing activity to determine off-target analysis, they are likely to be incomplete with regards to surveying potential off-target sites for prime editing systems. In particular, comparison of off-target editing for Cas9 nickases versus nucleases suggests that the nickase will have distinct off-target sites. Kim, D. Y., et. al. Nucleic Acids Res 48, 10576-10589 (2020).

Since the prime editing systems employ a Cas9 nickase, they are likely to have unique off-target profiles from the Cas9 nuclease with regards to potential active sites. In addition, the activity of prime editors is a function of the complementarity of the target primer binding site (PBS) and reverse transcriptase template (RTT) regions of the pegRNA to the genomic sequence—since a template mediated reaction is required for incorporation of new sequences into the genome, which cannot be mimicked by nuclease- or nickase-based off-target detection methods.

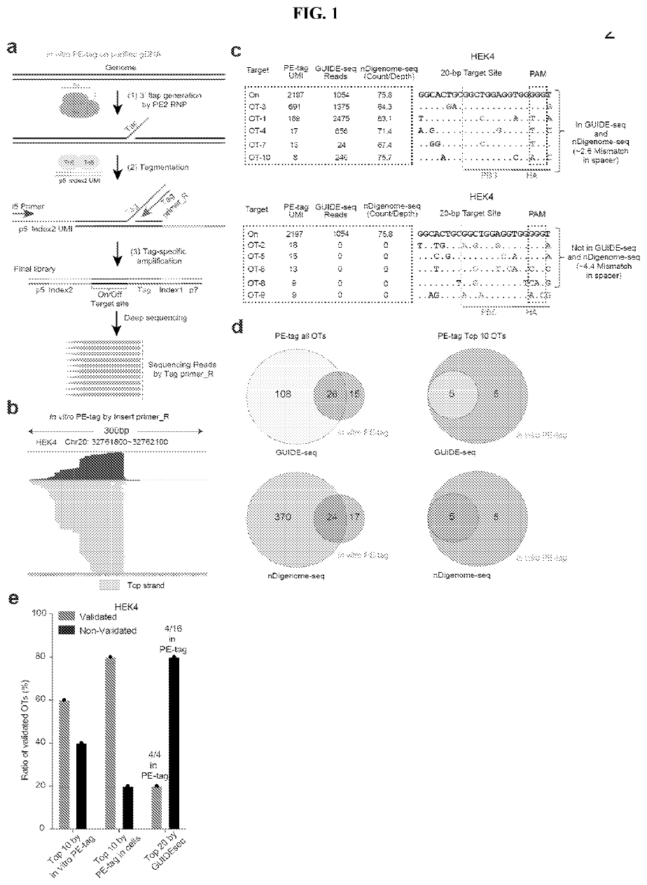

To address this unmet need for off-target assessment of prime editing systems, the present invention contemplates a PE-tag genome-wide off-target detection system. See, FIG. 1. The PE-tag technique comprises a purified prime editor protein and a pegRNA targeting the site of interest to introduce an amplification tag element into the genome that can be used to selectively amplify the genomic region containing the target site for an unbiased identification of each genomic locus that could be modified by a prime editor. A PE-tag method is most efficiently performed in vitro on purified genomic DNA, however, it can also be performed in cell culture or in vivo when delivered with suitable reagents.

The data presented herein demonstrates that PE-tag can identify off-target sites for prime editors for a variety of target sites including those with therapeutic application, which demonstrates the utility of this method at target sites suitable for clinical development. Further, there is a growing need for off-target analysis methods as studies move toward clinical trials. The FDA is becoming more demanding of extensive off-target analysis prior to allowing clinical trials to launch. See, SeQure Dx; sequre-dx.com. This is one example of a biotech start-up that is focused on providing off-target analysis methods for gene editing systems preparing for clinical trials.

PE-tag provides a readily accessible method for evaluating prime editor function genome-wide either in vitro or in cells. The present analysis of off target sites identified by only in vitro PE-tag (not by PE-tag in cells) suggests that most are inefficient sites for genome editing and consequently they have negligible indel rates when characterized. A higher false positive rate for in vitro PE-tag may be due to the use of a long reaction time (12 hrs) that preferentially increases the tag incorporation at inefficient editing sites in the genome. Although it is not necessary to understand the mechanism of an invention, it is believes that the pegRNA can be split into a separate sgRNA and RTT-PBS template, where an in trans template is used for off-target analysis to further simplify the performance of this approach. Two recent papers described alternate prime editor tag-based off-target detection methods in cells: i) PEAC-seq (Yu et al., “PEAC-seq adopts Prime Editor to detect CRISPR off-target and DNA translocation” Nat Commun 13:7545 (2022)), which uses a Cas9 nuclease-RT fusion to define off-target sites for nuclease-based editing systems; and ii) TAPE-seq (Kwon et al., “TAPE-seq is a cell-based method for predicting genome-wide off-target effects of prime editor” Nat Commun 13:7975 (2022)), which uses standard prime editing systems for off-target analysis, but does not incorporate Tn5 tagmentation that simplifies library construction.

Because PE-tag is a robust method for off-target site detection, it can solve problems regarding the generation of validation data for gene and cell therapy preclinical studies and clinical trials for CRIPSR systems that use a reverse transcriptase or polymerase to rewrite genomic sequence in a template-directed manner.

### I. Conventional Cas9 Off-Target Assessments

One of the primary concerns with the therapeutic application of genome editing systems for somatic cell genome editing is the potential for creating unwanted edits within the genome. These off-target effects can potentially change gene expression programs or lead to genomic rearrangements that can be destabilizing to the genome. Currently, there are proposed approaches for determining the off-target sites for nuclease-based genome editing systems. Bao et al., Nat Protoc 16, 10-26 (2021). For prime editor (PE) systems, however, these nuclease based approaches for determining the off-target sites are not directly applicable, since prime editing systems do not generate double-strand breaks (DSBs) in the genome. Direct or indirect DSB detection is central to conventional nuclease-based genome-wide off-target analysis methods.

Consequently, efforts to determine genome-wide off-target sites for PE systems have focused on either: 1) computational predictions of near-cognate sequences to the spacer sequence that is encoded by the pegRNA; or 2) genome-wide off-target analysis for a nuclease or nickase programmed with an sgRNA recognizing the same target site as the pegRNA. Anzalone, A. V. et al. Nature 576, 149-157 (2019); Chen, P. J. et al. Cell 184, 5635-5652.e29 (2021); Nelson, J. W. et al. Nat Biotechnol 1-9 (2021); and Kim, D. Y., et. al. Nucleic Acids Res 48, 10576-10589 (2020).

Since these nuclease and nickase methods are not utilizing PE platforms for off-target analysis, they are likely to be incomplete with regards to surveying potential off-target sites for prime editing systems. In particular, comparison of off-target editing for Cas9 nickases versus Cas9 nucleases suggests that Cas9 nickase have distinct off-target sites. Kim, D. Y., et. al., Nucleic Acids Res 48, 10576-10589 (2020). Since PE systems employ a Cas9 nickase, they are also likely to have unique off-target site profiles as compared to a catalytically active Cas9 nuclease.

### II. Prime Editor Off-Target Site Assessments

To address the above unmet need for off-target site assessment of PE systems, one embodiment of the present invention contemplates a PE-tag genome-wide off-target site detection system. See, FIG. 1. For example, a purified PE protein and a pegRNA targets a site of interest and appends or introduces an amplification tag element (e.g., tag) into the genome. This amplification tag element then hybridizes to an amplification primer to selectively amplify a genomic region that may comprise a prime editor recognition sequence to facilitate an unbiased identification of each genomic locus within that region which could be edited by a prime editor.

PE-tag can be efficiently performed either in vitro on purified genomic DNA or in cell culture. The data presented herein demonstrates that a PE-tag system identifies off-target sites for PE proteins at a variety of target sites. Many of these target sites are associated with therapeutic applications, which demonstrates the utility of this method at sites suitable for clinical development.

In one embodiment, the present invention contemplates a method for performing a PE-tag analysis to provide a preclinical development of prime editing tools for discovering on-target and functional off-target prime editor recognition sites associated with the correction of a pathogenic mutation. By assessment of the number of distinct UMI sequences that are incorporated at each site (on-target and off-target) the relative activity of the off-target sites can be ranked. Subsequently, off-target editing rates at any active off-target site can be ameliorated by engineering an appropriate prime editor protein and/or pegRNA. Although it is not necessary to understand the mechanism of an invention, it is believed that PE-tag is a robust system for off-target detection of PE activity.

### A. Overview

Prime editor fusion proteins generally comprise a Cas9 nickase protein and a reverse transcriptase (RT) protein which generates a nick in a non-target strand of the pegRNA-directed target site. Consequently, the 3′ end of the nicked DNA strand anneals to a primer binding site (PBS) sequence encoded at the 3′ end of the pegRNA. Once annealed, the 3′ end of the nicked DNA strand is extended using the reverse transcriptase protein derived from a template sequence encoded in a RT template (RTT) region of the pegRNA. Typically, the RTT region comprises a region that is homologous to the genome. This region of homology allows an efficient incorporation of the RT generated sequence within the genome via cellular DNA repair pathways. Anzalone, A. V. et al. Nature 576, 149-157 (2019).

Using this basic methodology, prime editor systems insert, delete or change DNA sequences within a genome. In one embodiment, the present invention contemplates a PE fusion protein complexed with a target-specific pegRNA comprising an a PBS and amplification tag element encoded within the 3′ portion of the pegRNA, and a spacer sequence. In one embodiment, the amplification tag element is appended to one strand of the genome. In one embodiment, the amplification tag element incorporates into a genome. In one embodiment, the incorporated or appended amplification tag element hybridizes to an amplification primer to amplify the target site and the neighboring genomic region. In one embodiment, the incorporated or appended amplification tag element hybridizes to an amplification primer to amplify other genomic sites with prime editor activity and their neighboring genomic regions. In one embodiment, the other genomic sites are a genomic regions where the prime editor has off-target editing activity. In one embodiment, the editing activity of the target sites are on-target editing activity. In one embodiment, the editing activity of the other genomic sites are off-target editing activity.

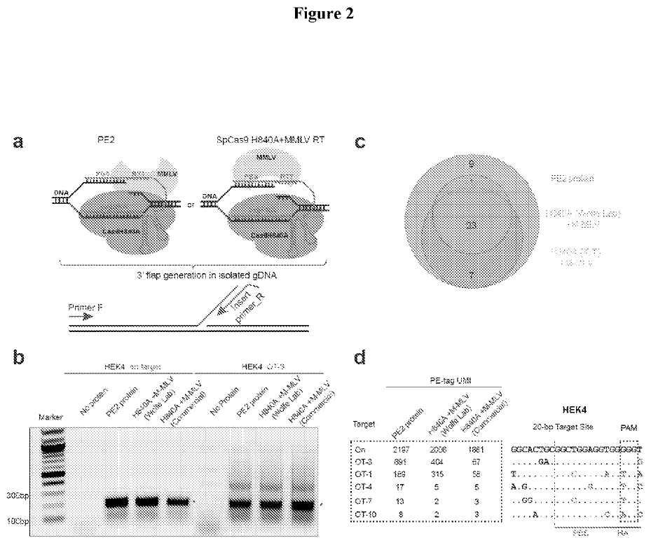

Consequently, a PE-tag system as contemplated herein can be run in vitro in a completely biochemical reaction or in cell culture to identify both on-target and off-target editing in the cellular environment. In addition, the PE-tag protocol can be run as a modular system wherein a Cas9 H840A nickase and an MMTV RT are separate proteins. Three different protein combinations were able to incorporate the amplification tag element into a target site demonstrating that the MMLV RT can function in trans to the Cas9 nickase for in vitro reactions. See, FIG. 2.

### B. Protocol

In one embodiment, the present invention contemplates a method providing a PE-tag system comprising an amplification tag element and inserting the amplification tag element at, or near, a pegRNA-targeted genomic region to amplify, sequence and identify on-target sites and/or off-target sites. Although it is not necessary to understand the mechanism of an invention it is believed that the method can be described in reference to three stages; i) amplification tag element incorporation; ii) universal adaptor incorporation; and iii) polymerase chain reaction amplification.

### 1. Amplification Tag Element Incorporation

Briefly, purified genomic DNA from a species of interest is treated with a purified prime editor protein (e.g., PE2) and a pegRNA for targeting a genomic sequence of interest and contains a target-specific PBS, an RTT region encoding an amplification tag element.

An exemplary amplification tag element has a length of twenty (20) bases (e.g., GTTGTCCGCTGTCACGACTC; (SEQ ID NO:1). Factors for selecting an amplification tag element sequence include, but are not limited to: 1) the absence of near-cognate sequences in the isolated gDNA; 2) a sequence (e.g., 20 nucleotide bases) comprising a primer annealing site with, for example, an ~50% GC content having an ~60 degree Tₘ for the amplification of the target site and its neighboring genomic regions where the amplification tag element has been appended or inserted. It should be apparent to one of skill in the art that many sequences can serve as the amplification tag element and does not have to be 20 bp in length. As long as the amplification tag element comprises a priming annealing site, it should be functional.

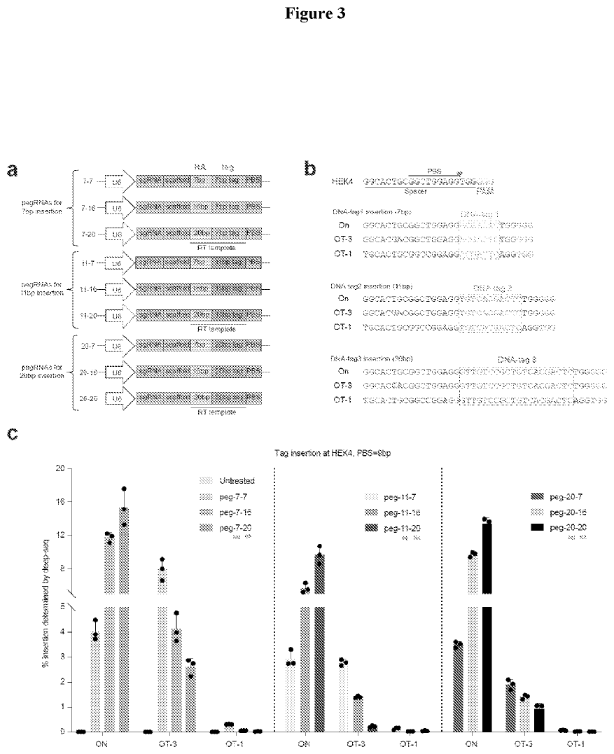

The data herein presents a variety of structural modifications to the pegRNA PBS-RT on their effect on amplification tag element insertion rates in cells (e.g., in HEK293T cells). See, FIG. 3. For example, nine (9) different pegRNAs were generated with amplification tag elements having differing lengths of homology arms (i.e., 7 bp, 11 bp or 20 bp). See, FIG. 3A. HEK site 4 (HEK4 locus) was selected as a testing target because it was previously reported with defined off-targets for prime editing systems. Anzalone, A. V. et al. Nature 576, 149-157 (2019). On-target and off-target editing efficiencies were measured by deep amplicon sequencing after co-transfecting pegRNA paired with PE2 plasmid DNA. See, FIG. 3B.

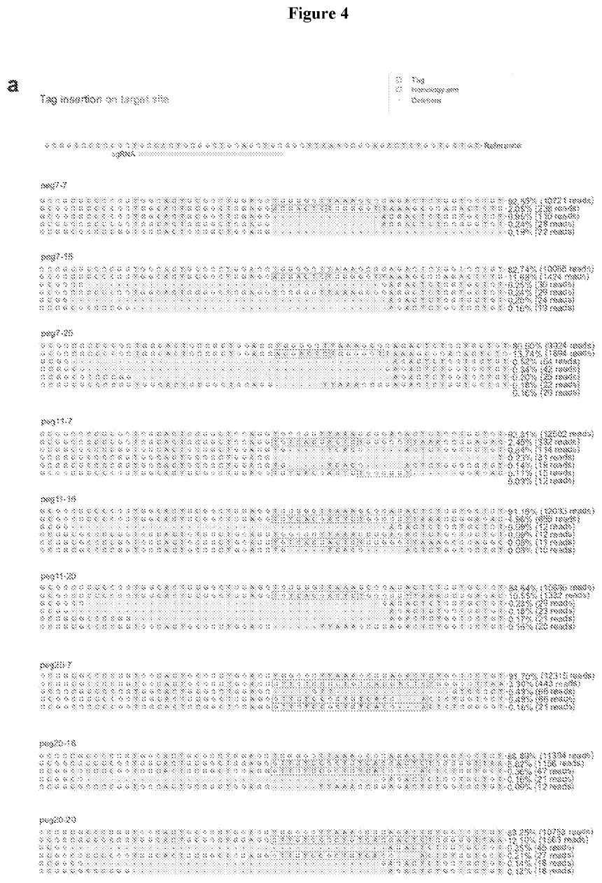

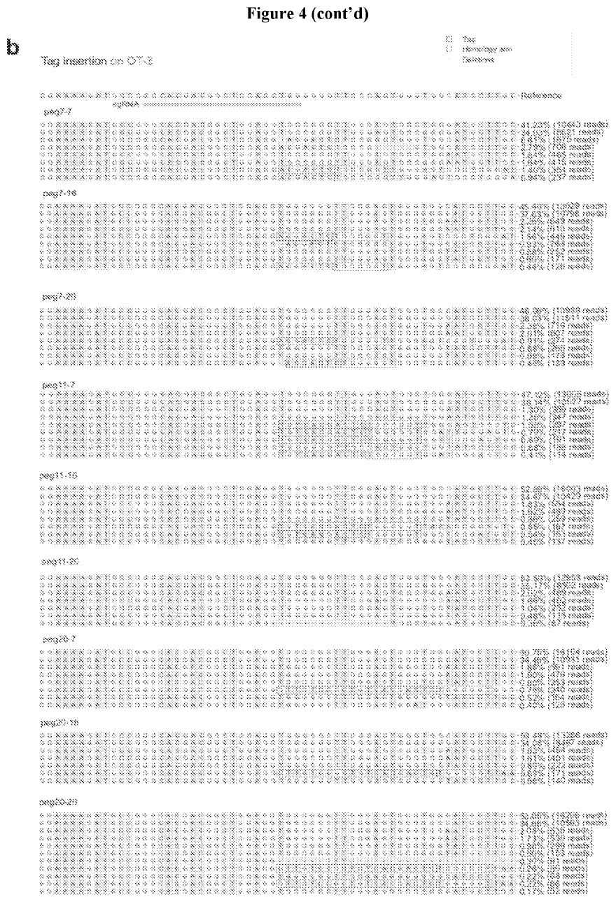

Amplification tag element insertions were observed with all tested pegRNAs in the on-target site and two active off-target site. It was observed that increasing the length of the homology arm in the RT template from 7 bp to 20 bp dramatically enhanced the insertion efficiency (average 4-fold) on a target site whereas the off-target insertion efficiency was decreased (average 2.5-fold). Also observed was that the editing efficiencies of prime editing on the off-target (OT)-1 site were much lower than OT-3 site, which is probably due to more mismatches in the spacer sequence and a mismatch located in the target-specific PBS region. See, FIG. 3B, C and FIG. 4A, B.

Collectively, these results indicate that a PE-mediated amplification tag element insertion at on-target sites and at off-target sites perform with an efficiency that is a function of the pegRNA length. A pegRNA comprising a twenty (20) nucleotide amplification tag element and a seven (7) nucleotide homology arm (i.e., a pegRNA 20-7) within an RTT region appeared to incorporate the amplification tag element with the best efficiency for both insertion and amplification of the target sites. See, FIG. 1A and FIG. 3.

### 2. Universal Adaptor Incorporation

A Tn5 transposase is used to tagment the purified genomic DNA. The tagmentation process incorporates sequencing adaptors (i.e., for example, an Illumina sequencing adaptor) throughout the genome in the form of a universal adaptor, wherein the universal adaptor encodes: i) a unique molecular identifier (UMI); ii) a pooling index; and iii) a i5 primer site into the genomic DNA which also contains a prime editor sequencing tags at active genomic sites. Although it is not necessary to understand the mechanism of an invention, it is believed that the Tn5 transposase allows much more efficient incorporation of the UMI-containing adaptors relative to traditional end repair approaches after shearing of the DNA. See, FIG. 1A. Giannoukos, G. et al. UDiTaS™, a genome editing detection method for indels and genome rearrangements. BMC genomics 19, 212 (2018).

### 3. Polymerase Chain Reaction Amplification

PE-tag amplification tag element integration sites in genomic DNA are captured using unbiased polymerase chain reaction (PCR) amplification between a Tn5-universal adaptor i5 primer binding site and an inserted amplification tag element. These amplicons are then sequenced by next-generation sequencing and mapped to the genome to identify the locations of prime editor activity. Unique prime editor events were counted individually using the UMI barcode information encoded in the amplified sequence. See, FIG. 1A. In one embodiment, a 5′ flanking region, relative to an on-target or off-target edit, is captured by unbiased PCR amplification.

### C. Summary: Proof of Principle

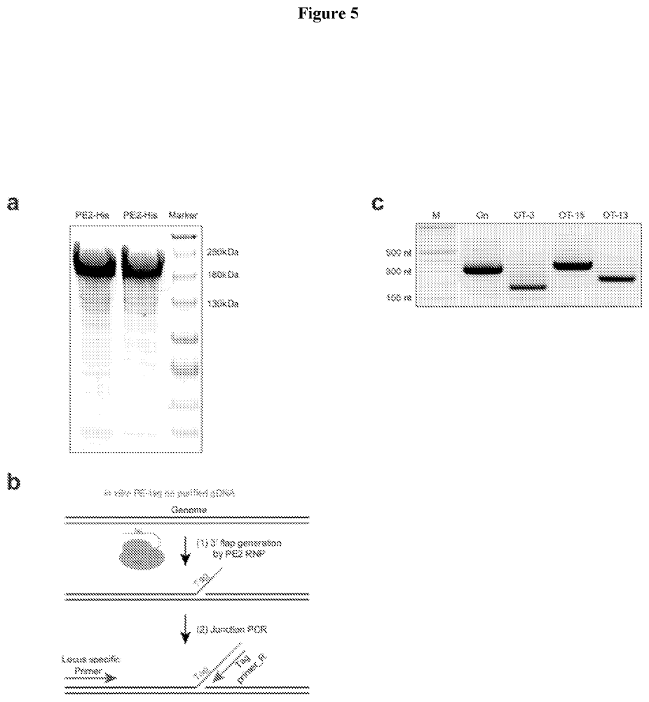

In one embodiment, a fusion PE protein and a pegRNA 20-7 incorporates an amplification tag element into a target site (e.g., a HEK4 locus). To validate the amplification tag element insertion, junction PCR was performed with site-specific forward (F) primers complementary to neighboring genomic regions for one on-target site and three off-target sites and a reverse (R) primer that hybridizes to the inserted amplification tag element. Successful amplification of these genomic regions was demonstrated to show that a PE-tag method can direct an amplification tag element at both on-target sites and off-target sites. See, FIG. 5A, 5B. These data suggest that PE-tag can be used to determine an unbiased genome-wide on-target site and off-target site analysis of active prime editor recognition sites.

### D. PE-tag Analysis of Prime Editor Gene Editing Activity

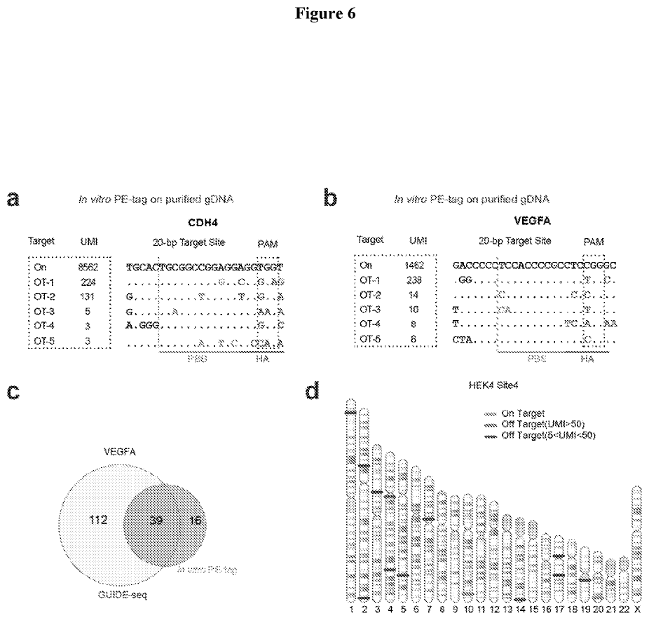

An in vitro PE-tag analysis was performed with a PE2 fusion protein and three different pegRNAs, each targeted to a different endogenous gene in isolated gDNA from HEK293T cells. An analysis of the amplification tag element integration sites identified precise genomic locations of prime editor gene editing activity induced by each of the PE2/pegRNA complexes. See, FIGS. 1B-1D and FIG. 6. For a majority of these edited genomic sites, an overlapping target sequence was identified that was either a fully-cognate on-target site or a near-cognate off-target site. See, FIGS. 1C & 1D and FIG. 6A-6B.

Substantial differences were observed between the total number of potential off-target sites detected by in vitro PE-tag at the HEK4 target site and the number of potential off-target sites identified by GUIDE-seq for SpCas9 or nDigenome-seq for SpCas9 H840A. Tsai, S. Q. et al. GUIDE-seq enables genome-wide profiling of off-target cleavage by CRISPR-Cas nucleases. Nat Biotechnol 33, 187-197 (2015). Kim, D. Y., Moon, S. B., Ko, J. H., Kim, Y. S. & Kim, D. Unbiased investigation of specificities of prime editing systems in human cells. Nucleic Acids Res 48, 10576-10589 (2020). This is presumably due to the difference that PBS annealing of the nicked non-target strand for RT extension by the prime editor is captured by PE-tag but not by GUIDE-seq. This suggests that genome-wide off-target site analysis for a Cas9 nuclease programmed with an sgRNA does not accurately reflect or predict the off-target sites induced by prime editors. See, FIG. 1D.

Though prime editor off-target sites were found dispersed throughout the genome (e.g., in exons, introns and noncoding intergenic regions), a few off-target sites were identified by PE-tag that contain an average UMI number that is larger than fifty (e.g., VEGFA, HEK4 or CDH4). These UMI numbers indicate a high likelihood of being an active off-target site. See, FIGS. 1C & 1D; and FIG. 6A-6B, 6D.

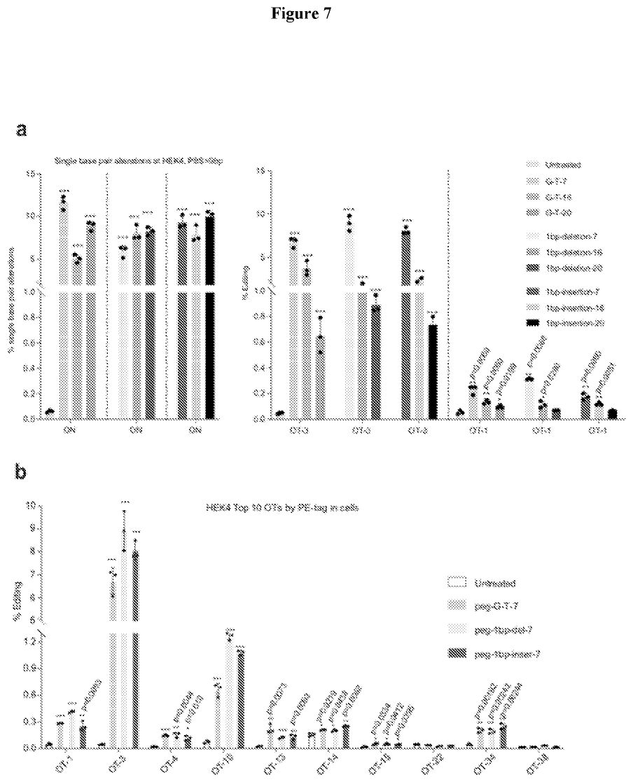

Prime editing activity other than insertions such as, nucleotide conversion or deletions, were also measured at these potential off-target sites. For example, nine different pegRNAs were generated to detect either nucleotide conversion, 1 bp-insertion or 1 bp deletion with different lengths of homology arms (e.g., 7 bp, 16 bp and 20 bp). The efficiency of editing by these PE-tag fusion protein complexes were determined by targeted amplicon deep sequencing in HEK293T cells after co-transfecting pegRNA paired with PE2 plasmid DNA. The data demonstrates prime editor targeted nucleotide conversion, insertions and deletions at both on-target sites and off-target sites. Editing was detected at 60% of the top 10 off-target sites captured by in vitro PE-tag. Where examined, the editing rates at off-target sites are reduced as the length of the HA increases. It was also observed that the pegRNA RTT sequence was inserted into many off-target sites at a low rate with variable efficiency. For example, among the editing outcomes at HEK4 OT-10, the tag insertion rate is higher than deletion rate when a pegRNA 20-7 was used, while the deletion rate is much higher than the pegRNA templated insertion when a pegRNA designed for single nucleotide alteration was used. See, FIGS. 7A &7B. Together, these data suggest that PE-tag enables genome-wide quantification of prime editor gene editing activity at both on-target and off-target sites in vitro in isolated gDNA.

### E. Genome-Wide PE-tag Analysis

Because in vitro PE-tag cannot incorporate a 3′ sequence extension into the gDNA, only the PAM distal region (relative to non-target strand cleavage site) can be captured by a single unidirectional PCR amplification. In cells, DNA repair via 5′ flap excision and 3′ flap ligation provides the opportunity to capture both genomic regions flanking the position of amplification tag element incorporation by two separate unidirectional PCR amplifications.

In one embodiment, the present invention contemplates an unbiased PE-tag genomic analysis in vitro comprising: a) appending an amplification tag element at a PE-introduced gDNA single strand break (SSB or nick); b) incorporating a universal adaptor comprising a UMI, a pooling barcode, and a i5 primer site with a Tn5 transposase into the gDNA; c) performing one unbiased PCR amplification using an amplification tag element-specific reverse primer with a sequence adaptor forward i5 primer (i5_F+Tag_R) to capture a gDNA amplicon comprising the amplification tag element with its neighboring genomic loci; d) performing a second round of PCR amplifications to incorporate the required i5 and i7 adaptor sequence; and e) performing next-generation sequencing on the gDNA amplicon. See, FIG. 1A.

A PE-tag analysis was performed with gDNA from HEK293T cells transfected with PE2-ribonucleoproteins (RNPs) and three different pegRNAs targeted to different endogenous genes. The precise genomic locations of prime editing recognition sites were also identified. See, FIGS. 1B-1C and FIG. 6.

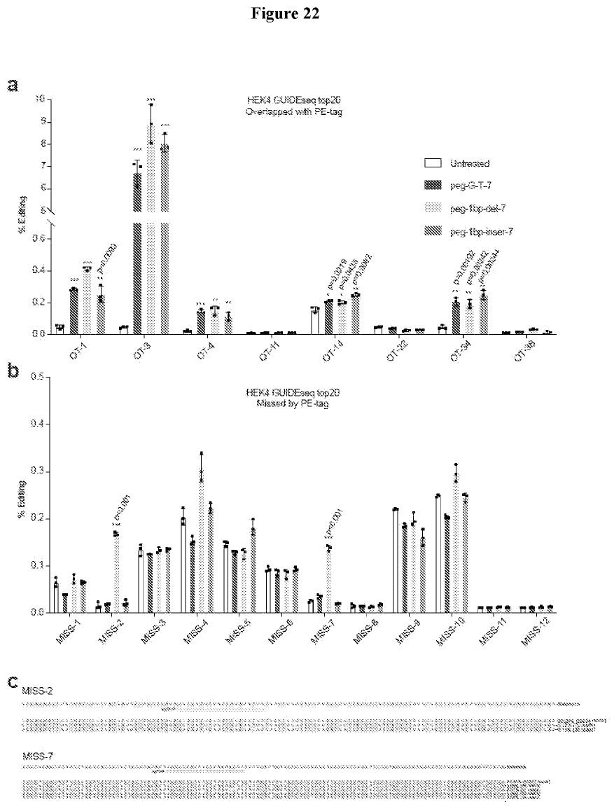

To evaluate the false negative rates for PE-tag, PE2 editing rates were assessed on the top 20 OTs from GUIDE-seq at the HEK4 locus. Only 4 OTs from the GUIDE-seq top 20 displayed consistent editing across the set of three HEK4 pegRNAs that were tested. These are all present in the top 10 OTs from in vitro PE-tag. FIG. 1E & FIG. 22A. For the twelve GUIDE-seq OTs that were not captured by PE-tag, significant editing was detected at 2 of 12 sites only when a pegRNA encoding a 1 bp deletion was employed. No significant editing was detected at these 12 OTs when a pegRNA encoding a G-T substitution or 1 bp-insertion was used. FIGS. 22B & 22C. Together, these data suggest that PE-tag has a low false positive and false negative detection rate.

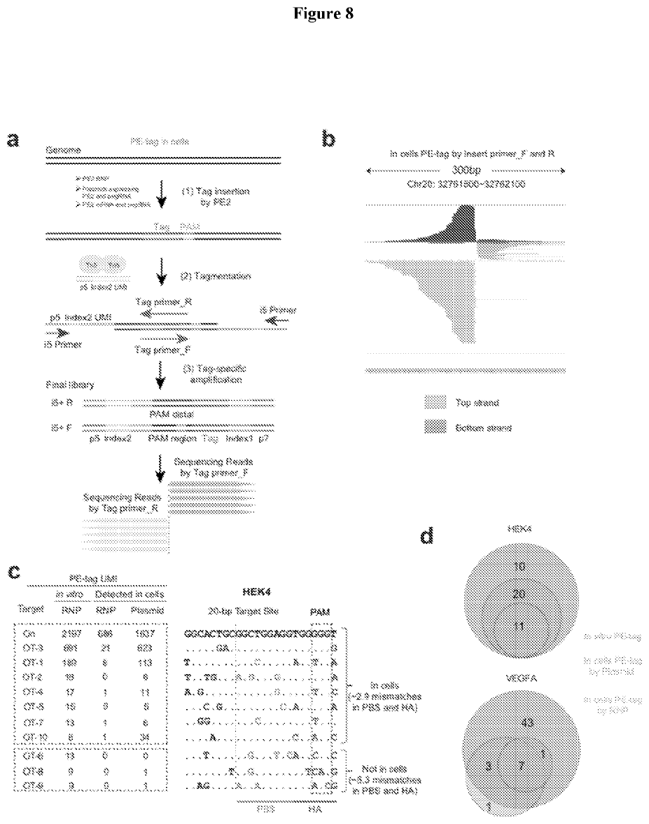

In one embodiment, the present invention contemplates an unbiased PE-tag genomic analysis in cells comprising: a) incorporating an amplification tag element at a PE-introduced gDNA single strand break (SSB or nick); b) ligating a 3′ flap that contains the amplification tag element into a gDNA; c) incorporating a universal adaptor comprising a UMI, a pooling barcode, and a i5 primer site with a Tn5 transposase into the ligated gDNA; d) performing two unbiased PCR amplifications using an amplification tag-specific forward primer or an amplification tag element-specific reverse primer with the sequencing adaptor-specific forward i5 primer (e.g., i5-_F+Tag_F, i5_F+Tag_R) on the ligated gDNA to capture a gDNA amplicon comprising the amplification tag element with its neighboring genomic loci; e) performing a second round of PCR amplifications to incorporate the required i5 and i7 adaptor sequence; and f) performing next-generation sequencing on the gDNA amplicon. See, FIG. 8A.

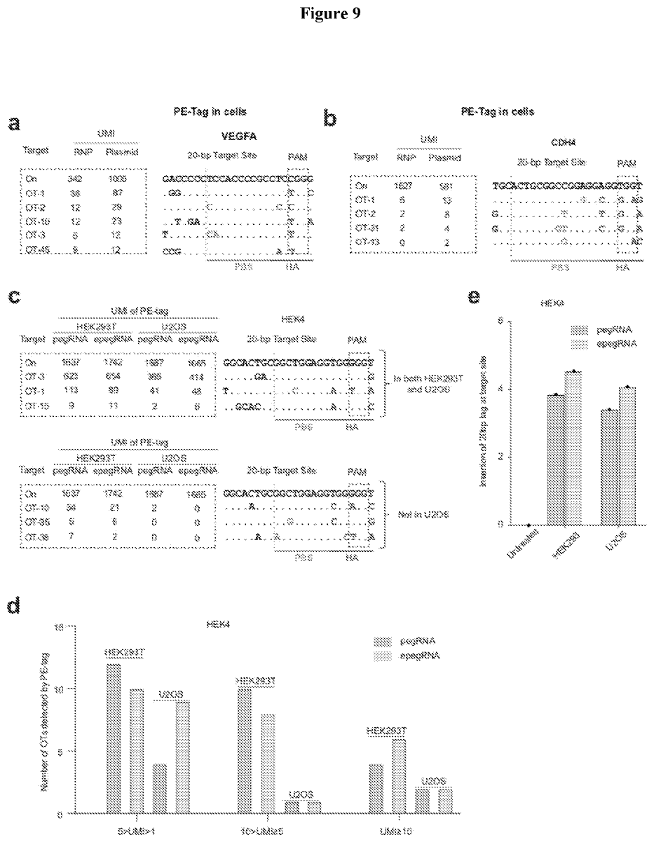

A PE-tag analysis was performed with HEK293T cells transfected with PE2-ribonucleoproteins (RNPs) or a PE-tag fusion protein encoded within a plasmid that express PE2 and three different pegRNAs targeted to different endogenous genes. Consistent with PE-tag in vitro on isolated gDNA (supra), the precise genomic locations of prime editor recognition sites in cells were also identified. See, FIGS. 8B-C and FIGS. 9A & 9B.

The total number of off-target sites detected by PE-tag in HEK293T cells was lower than the number of off-target sites identified by PE-tag in vitro in isolated gDNA. PE-tag in HEK293T cells by PE RNP administration resulted in identifying the lowest number of off-target sites, which is probably due to the short lifespan of PE RNP in cells. Putative off-target sites identified by PE RNP in cells include: i) HEK4; 11 of 41; and ii) VEGFA; 7 of 54 of the potential off-target sites captured by PE-tag in vitro in isolated gDNA. See, FIG. 8D.

A recent study demonstrated that mismatch repair deficiency may enhance prime editing. Chen, P. J. et al. Cell 184, 5635-5652.e29 (2021). It is noted that HEK293T cells are partially MMR deficient due to hypermethylation of the MLH1 promoter which may permit a higher number of off-target editing events when PE-tag is performed. Consequently, a PE-tag analysis was performed in MMR-competent U2OS cells with the same pegRNA and engineered pegRNA (epegRNA) used above for HEK 4 locus in HEK293T cells. Although the total number of detected off-targets (OTs) was reduced for both pegRNA and epegRNA, the off-targets identified in U2OS cells by PE-tag are overlapped with the OTs detected in HEK293T cells. See, FIGS. 9C, 9D & 9E. Interestingly, a similar number of potential OTs was observed between epegRNA and pegRNA in both HEK293T and U2OS cells. Together, these data suggest that PE-tag enables a genome-wide detection of prime editing in living cells.

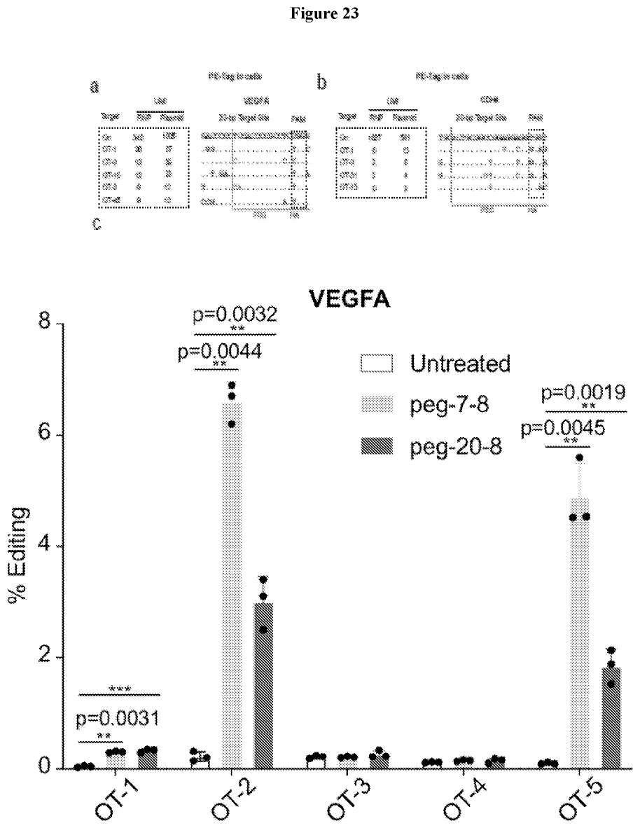

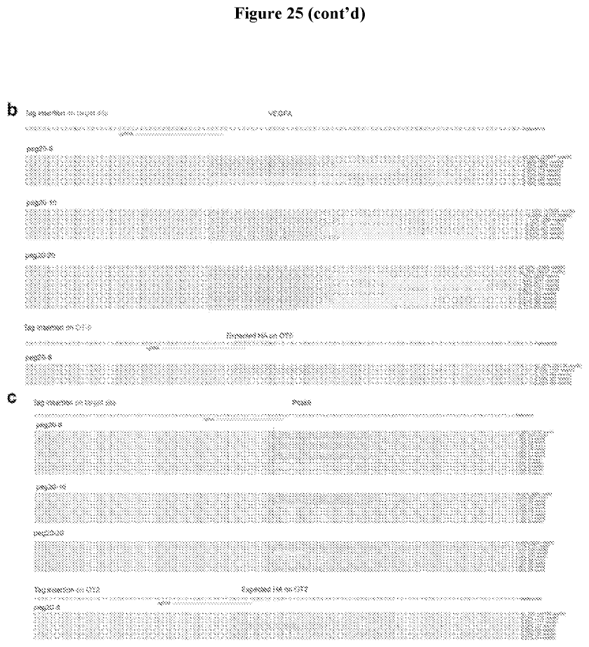

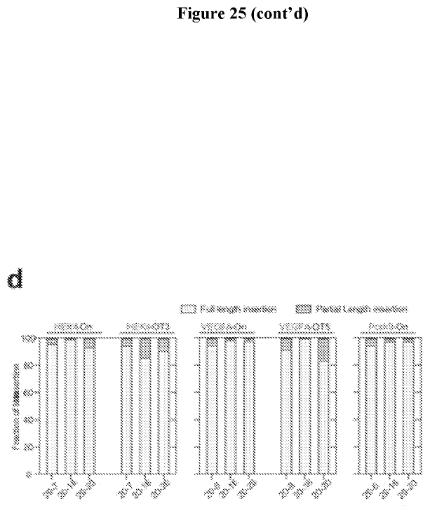

Performing PE-tag in cells provides the opportunity to capture both genomic regions flanking the position of tag incorporation by two separate unidirectional PCR amplifications. The efficiency of PE-tag in HEK293T cells was evaluated using a 20-7 tagging pegRNA at three different target sites (HEK4, VEGFA & CDH4) where the prime editing components were delivered by electroporation as RNPs or expression plasmids. The relative frequency of tag insertion versus indels at these target sites and two prominent off-target sites was determined by targeted amplicon deep sequencing. More than 60% of edits are tag insertions across these sites with more than 80% of the tag insertions being full length. FIGS. 25A-D. Like in vitro PE-tag on isolated gDNA, genomic locations of prime editing in cells were identified. FIGS. 23A & 23B. Notably, 7 of the top 10 potential OTs identified by in vitro PE-tag were also recovered by PE-tag in cells at HEK4. These potential off-target sites contain an average of 2.9 mismatches within the PBS and HA region of the pegRNA relative to the target site. Notably, the three potential OTs PE-tag in cells failed to capture contain an average 5.3 mismatches in their PBS and HA region of the pegRNA. Indels introduced by prime editing were validated at 3 of 5 tested VEGFA OT sites with indel rates ranging from 0.3% to 6.3%. FIG. 23C. For the HEK4 target site, PE2 induced indels (0.2% to 0.4%). H840A nickase also induced indels that ranged from 0.1% to 0.4%.

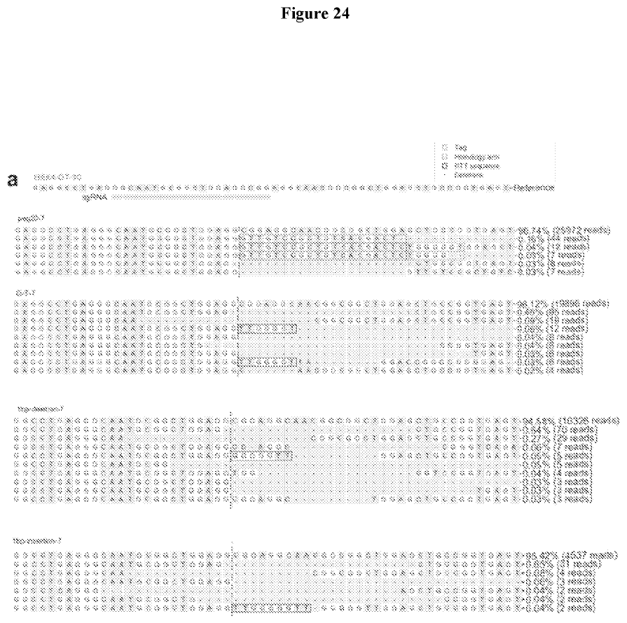

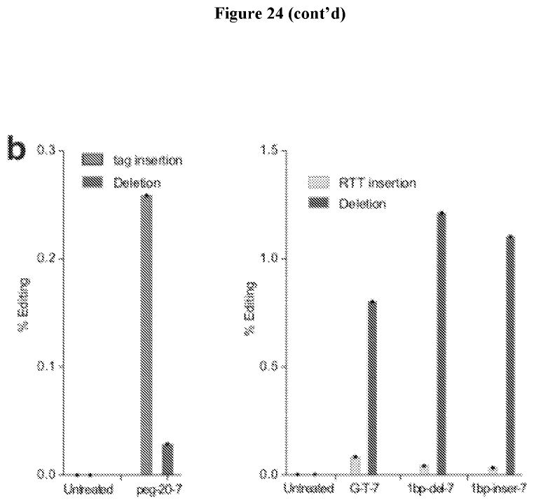

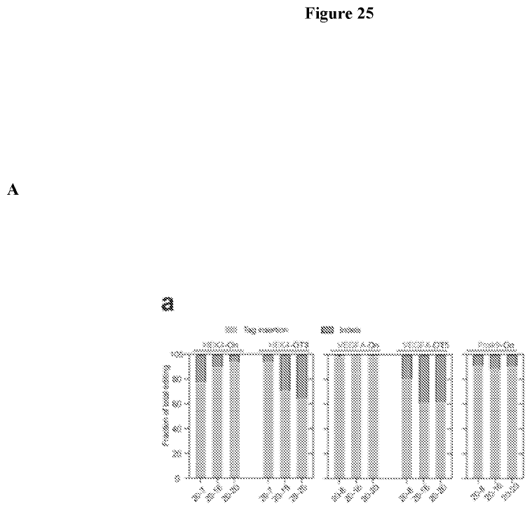

The pegRNA RTT sequence was inserted into many off-target sites at a low rate with variable efficiency. For example, among the editing outcomes at HEK4 OT-10, the tag insertion rate is higher than deletion rate when a pegRNA 20-7 was used, while the deletion rate is much higher than the pegRNA templated insertion when a pegRNA designed for single nucleotide alteration was used. FIGS. 24A & 24B.

These results are consistent with prior studies demonstrating the impact of multiple mismatches in either the PBS or HA region of the RTT on prime editing efficiency. The total number of off-target sites detected by PE-tag in HEK293T cells was lower than the number of off-target sites identified by PE-tag in vitro in isolated gDNA. PE-tag in HEK293T cells using prime editing components delivered as RNP produced the lowest number of recovered potential off-target sites, which is probably due to the lower editing activity of prime editing RNPs and their short lifespan. Putative off-target sites identified by PE RNP in cells include 11 of 41 (for HEK4) or 8 of 54 (for VEGFA) of the off-target sites captured by PE-tag in vitro on isolated gDNA.

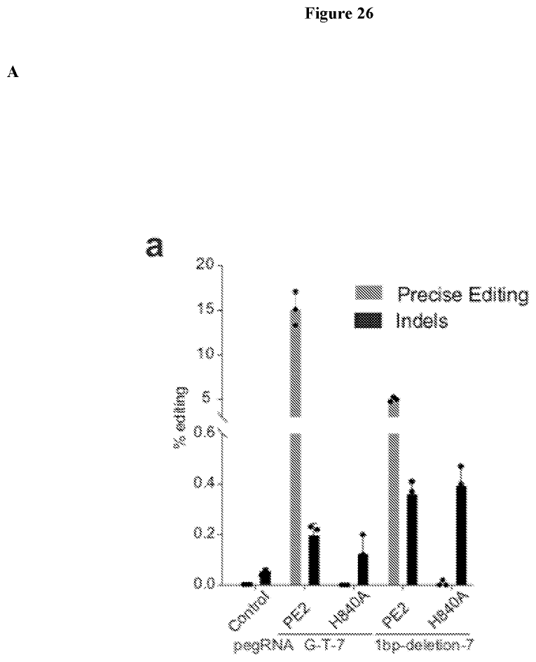

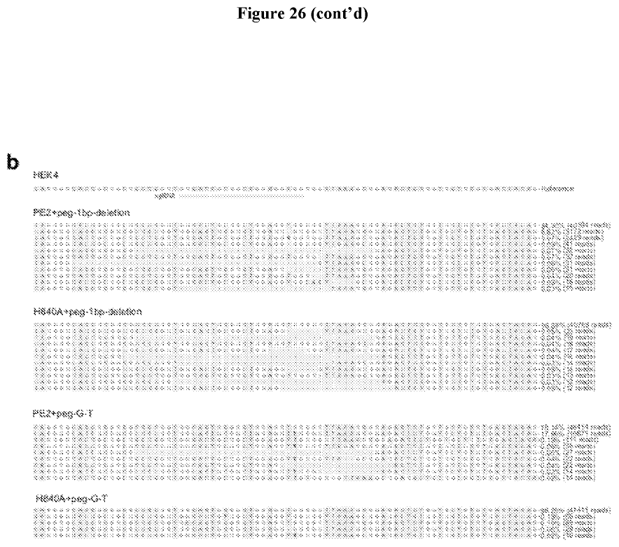

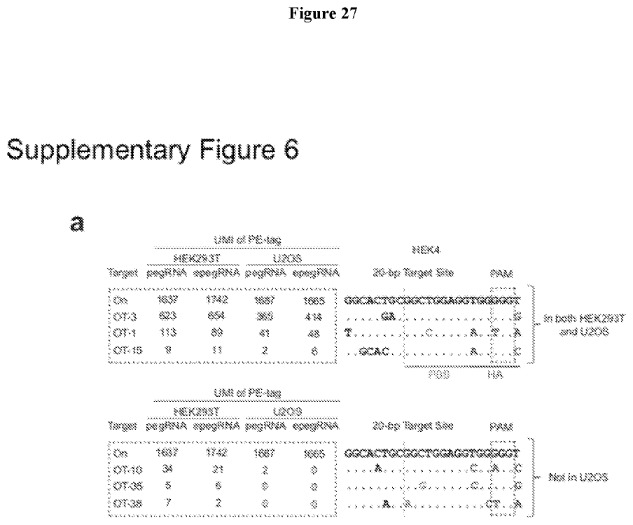

For the HEK4 target site, PE2 induced indels ranging between 0.2% to 0.4%. H₈₄₀A nickase also induced indels that ranged from 0.1% to 0.4%. FIGS. 26A & 26B.

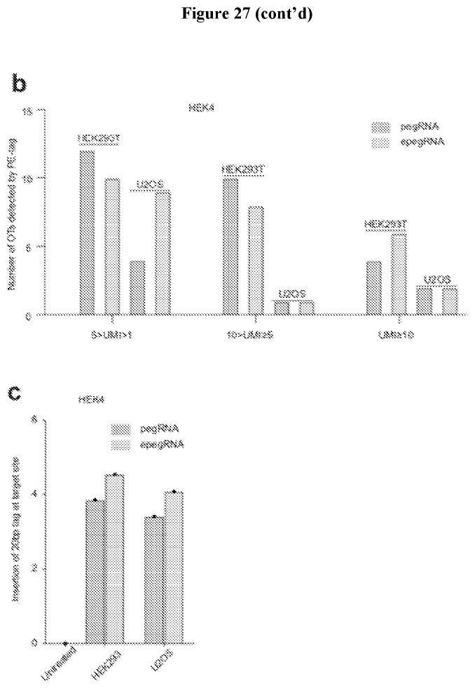

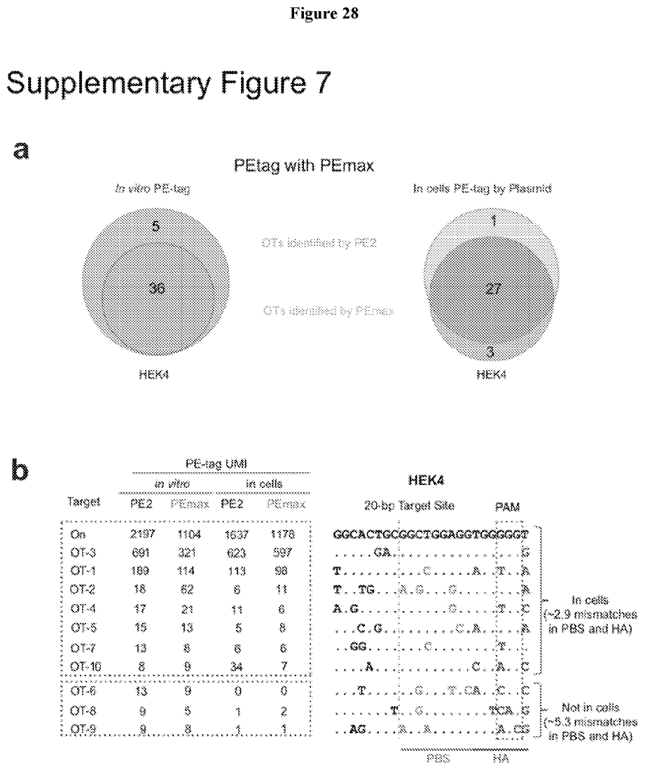

Mismatch repair (MMR) deficiency is known to enhance prime editing rates and HEK293T cells are partially MMR deficient. Consequently, to probe the influence of MMR on off-target prime editing, PE-tag was performed in MMR competent U2OS cells. The impact of the improved engineered pegRNA (epegRNA) design on PE-tag in HEK293T and U20S cells was evaluated. Although the total number of detected potential OTs was lower for both HEK4 pegRNA and epegRNA in U2OS cells, the OTs identified by PE-tag overlapped with the OTs detected in HEK293T cells. FIGS. 27A & 27B. Interestingly, a similar number of potential OTs for the epegRNA and pegRNA in both HEK293T and U2OS cells was also observed. FIG. 27C. In addition, off-target editing outcomes were examined for the PEmax system by PE-tag, which produced similar genome-wide editing profiles to PE2. FIGS. 28A & FIG. 28B.

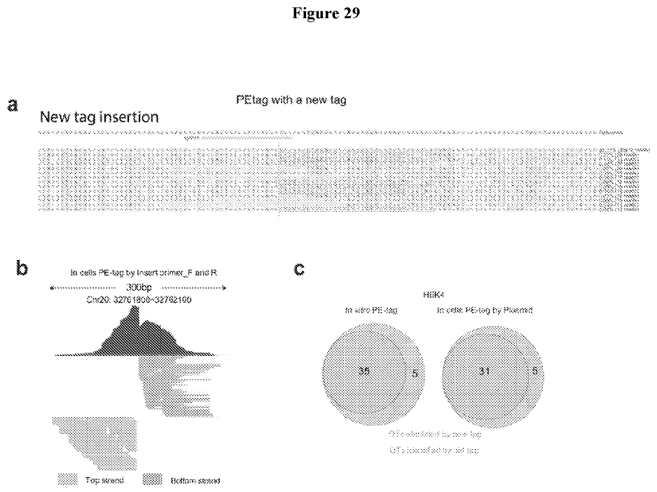

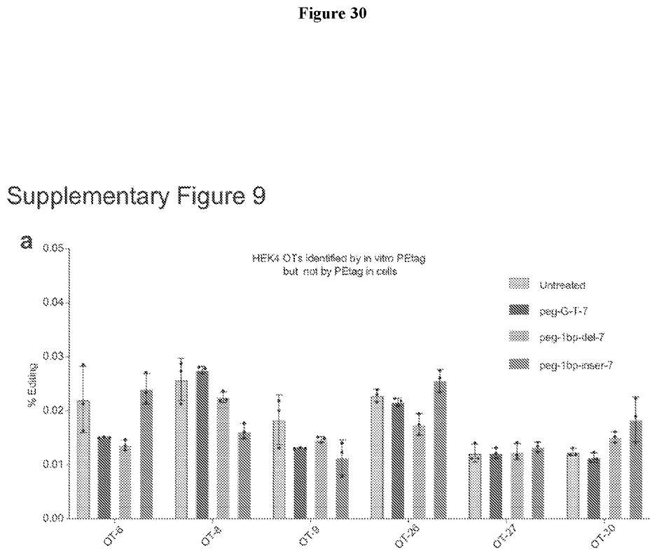

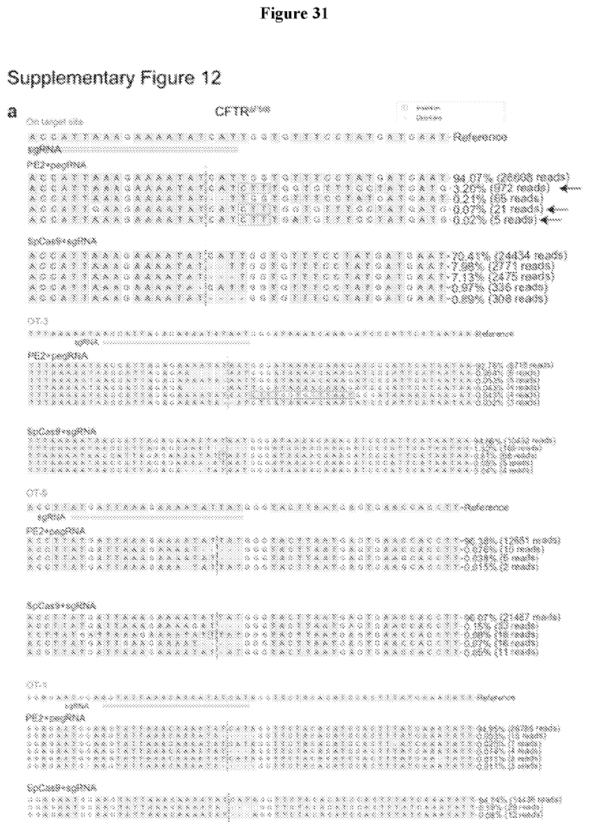

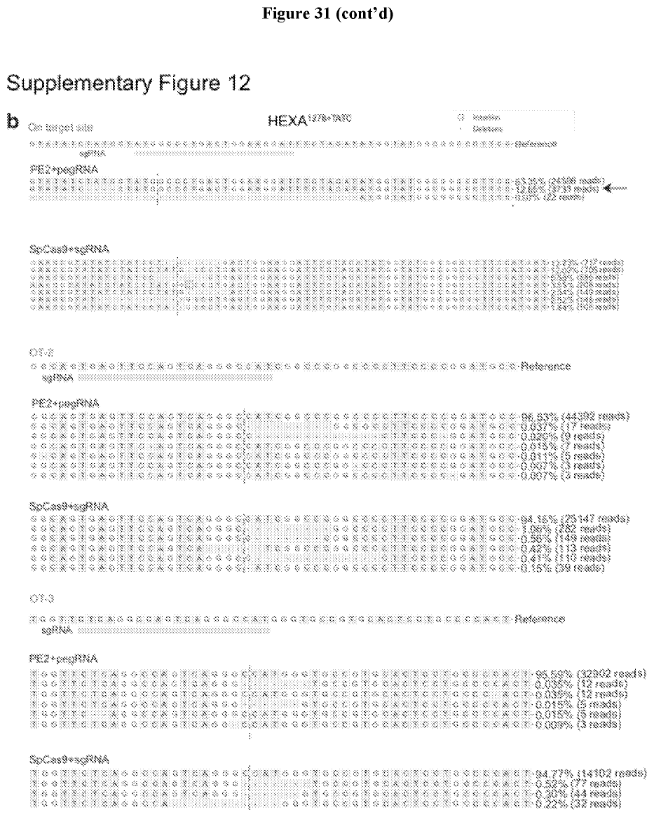

To test if a different tag sequence affects the insertion efficiency and off-target readout, in vitro PE-tag and PE-tag was performed in cells with a 27 nt tag using a pegRNA targeting the HEK4 locus. Notably, OTs were identified by in vitro PE-tag and PE-tag in cells using an amplification tag sequence that completely subsumes OTs captured with a different tag. FIGS. 29A, 29B & 29C. Together, these data demonstrate that PE-tag enables genome-wide detection of prime editing activity for a variety of different prime editing systems in a variety of different cell types.

### F. In Vivo Mouse Liver PE-tag Analysis

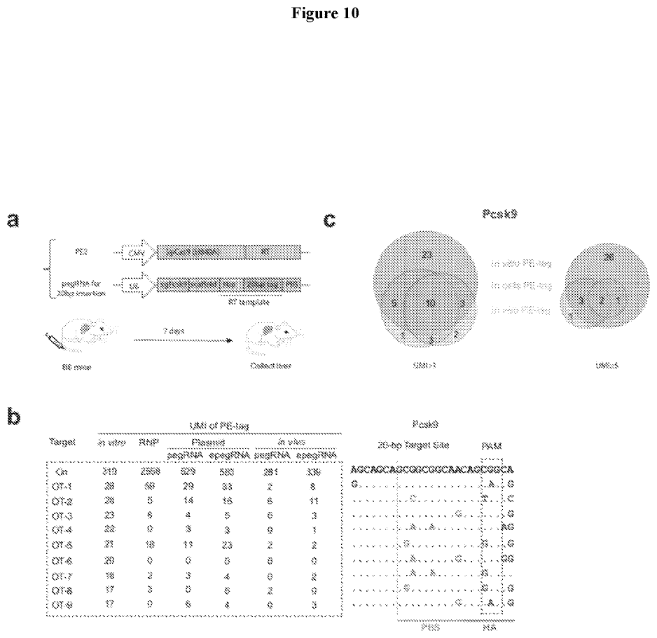

An in vivo PE-tag analysis was evaluated in mouse liver at the Pcsk9 genomic site. For this target site, expression vectors for PE2 and a pegRNA were co-delivered by hydrodynamic tail vein injection (HTVI) and expressed to insert a 20 bp sequencing tag. PE-tag was then performed on the liver of these mice sacrificed at day 7 post injection. See, FIG. 10A. To directly compare in vitro PE-tag with in vivo PE-tag at the same target site, PE-tag was performed in multiple formats in Hepa1-6 cells: 1) PE2 RNPs and 2) PE2 expression plasmids with a pegRNA or epegRNA. In vitro PE-tag was performed on isolated gDNA from Hepa1-6 cells. See, FIG. 10B. PE-tag amplicon libraries were prepared from different genomic DNA and sequenced. Using a conservative UMI threshold (e.g., ≥5), three (3) off-target sites were identified with in vivo delivery, six (6) off-target sites were identified with plasmid delivery and thirty-two (32) off-target sites were identified with isolated gDNA. See, FIG. 10C.

It was observed that in vitro PE-tag in isolated gDNA or PE-tag in cells results in more discovered off-target sites than the PE-tag in vivo in mouse liver based on UMI counts. See, FIGS. 10B, C. These differences are probably due to a lower prime editing efficiency in vivo. Overall, these data demonstrate that PE-tag provides a straightforward approach to discover prime editor recognitions sites and/or detect genome-wide prime editing in vivo.

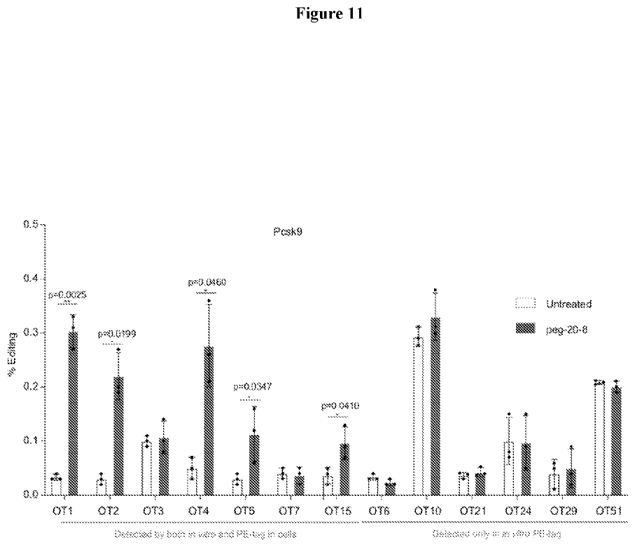

Prime editing activity was validated at five OT sites in Hepa1-6 cells with indel rates ranging from 0.1% to 0.3%. Editing was undetectable at six examined OTs captured by in vitro PE-tag but not captured by PE-tag in cells. See, FIG. 11. Validated prime editing activity was determined at five OT sites in Hepa1-6 cells with indel rates ranging from 0.1% to 0.3%. FIG. 10D. Editing at six examined OTs captured by in vitro PE-tag but not captured by PE-tag in cells was undetectable. FIG. 10D.

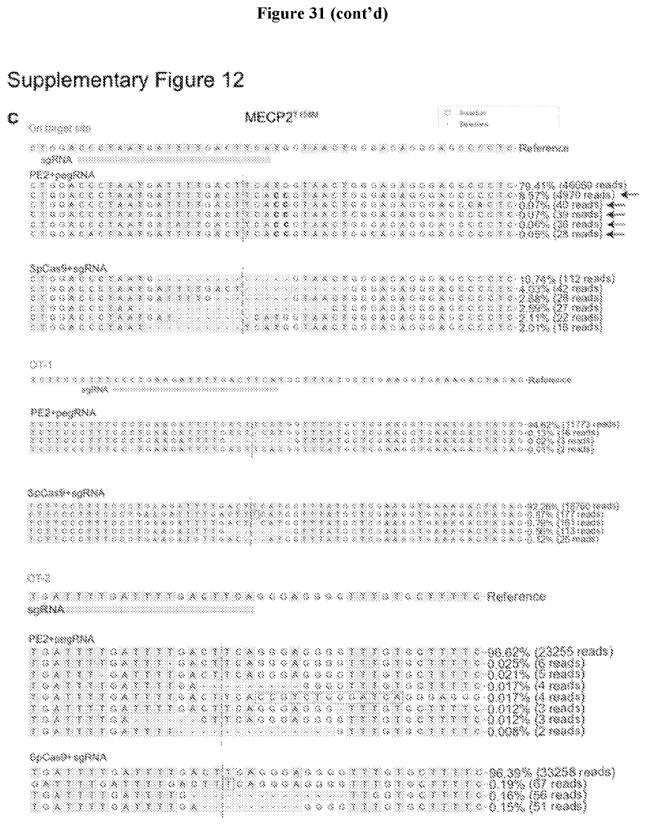

Editing was also not detected at six potential OTs for the HEK4 pegRNA that were only captured by in vitro PE-tag. FIG. 30. The differences in the number of captured potential OTs for each PE-tag method likely stem from the lower prime editing efficiency in vivo and the requirement for substantial HA homology for tag incorporation for PE-tag performed in cells or in vivo. Many of the indels at these OT sites are small deletions around the putative cleavage site, suggesting that they may be products of the PE nickase activity, as opposed to template-dependent changes based on the sequence encoded within the RTT segment of the pegRNA. FIGS. 31A-C.

### G. Pathogenic Mutations

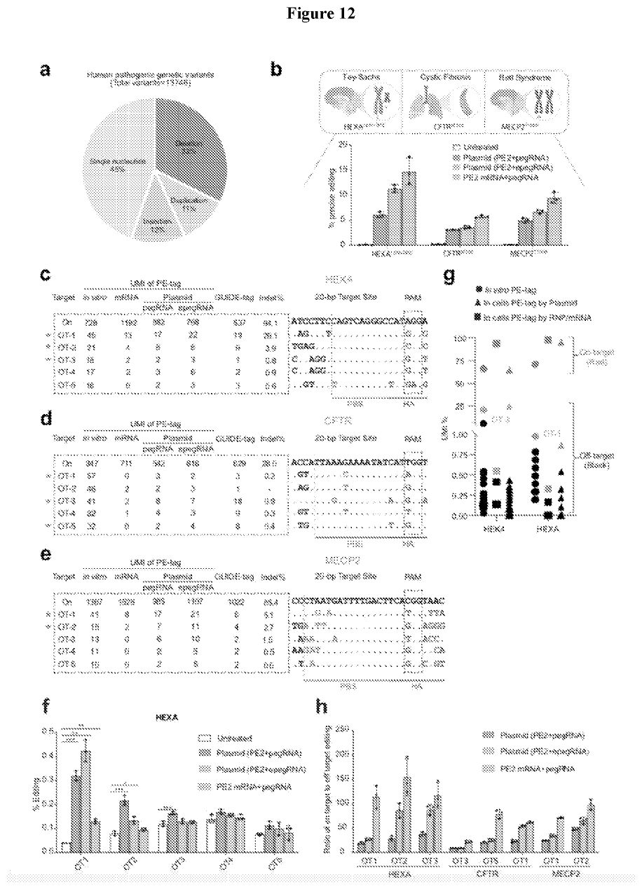

In one embodiment, the present invention contemplates a method to detect off-target prime editor recognition sites associated with genomic regions comprising pathogenic alleles. See, FIG. 12A. In one embodiment, PE-tag detects genome-wide off-target sites induced by pegRNAs used for correction of genetic variants associated with human diseases (e.g., a therapeutic pegRNA).

The data presented herein demonstrates the ability of PE-tag to identify potential off-target sites that are associated with therapeutic pegRNAs to correct common mutations for three different diseases: Tay-Sachs, Rett Syndrome and Cystic Fibrosis are evaluated.

The most common mutation that causes Tay-Sachs disease is believed to be a 4-bp insertion in HEXA (HEXA^{1278+TATC}). Deletion of phenylalanine 508 of the cystic fibrosis transmembrane conductance regulator (CFTR^{ΔF508}) is a known cause of cystic fibrosis. Rett syndrome is believed to be caused by mutations in methyl CpG binding protein 2, where one common point mutation is T158M (MeCP2^{T158}M). See, FIG. 12B. Prime editors with therapeutic pegRNAs have been shown to be able to correct two of the above mutations. Anzalone, A. V. et al. Nature 576, 149-157 (2019); and Geurts, M. H. et al. Life Sci Alliance 4 (2021).

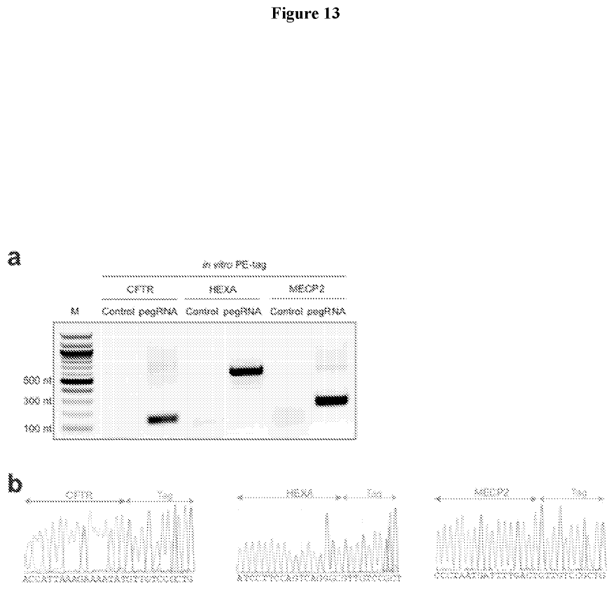

Therapeutic pegRNAs were designed for correction of the MeCP2^{T158}M mutation, the HEXA^{1278+TATC} mutation and the CFTR^{ΔF508} mutation. A PE-tag analysis was performed with both an amplification tag element pegRNA (to identify off-target sites) and these therapeutic pegRNAs to correct above three pathogenic mutations was tested: i) in vitro with isolated gDNA; ii) in cells with plasmid delivery; and iii) in cells with mRNA delivery. See, FIGS. 12C-E. Amplification tag element insertions at a target loci were confirmed by junction PCR and Sanger sequencing. See, FIGS. 13A, B. As a benchmark for a PE-tag off-target analysis approach, potential off-target sites for SpCas9 RNP were determined with an sgRNA containing the same spacer sequence as each pegRNA and were compared using GUIDE-tag analysis.

Using a relaxed UMI threshold (e.g., ≥1) for in vitro PE-tag, sixty-two (62) CFTR off-target sites; forty-three (43) HEXA off-target sites; and sixty-five (65) MECP2 off-target sites were identified. Using a conservative UMI threshold (e.g., ≥5) in cells using in vitro, plasmid or mRNA delivery, three (3) CFTR off-target sites; four (4) HEXA off-target sites; and three (3) MECP2 off target sites were identified.

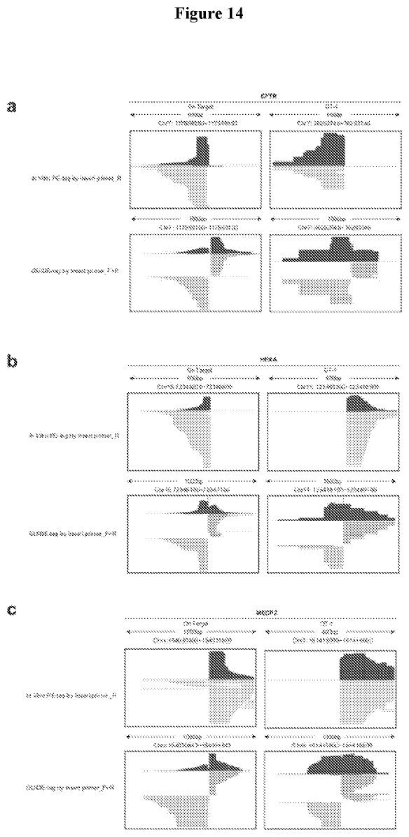

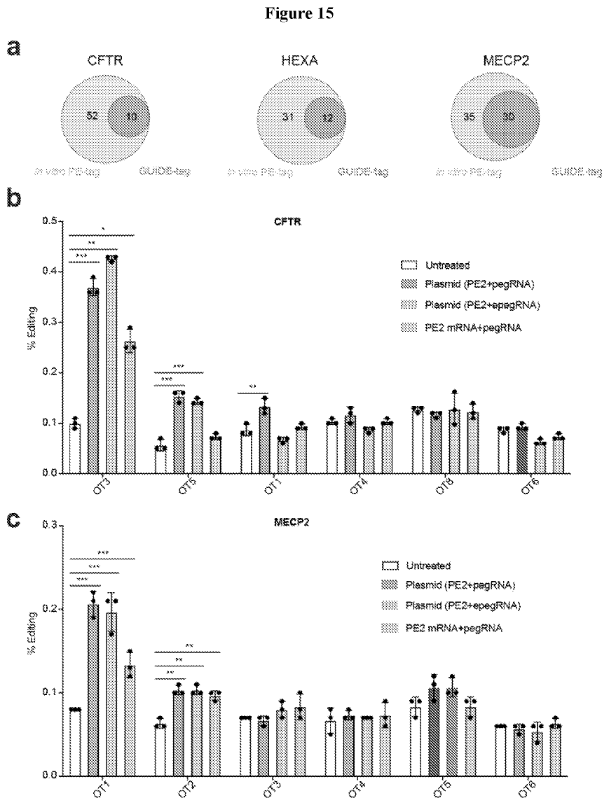

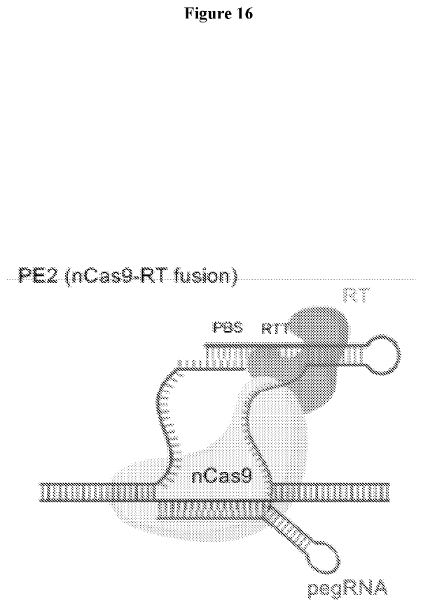

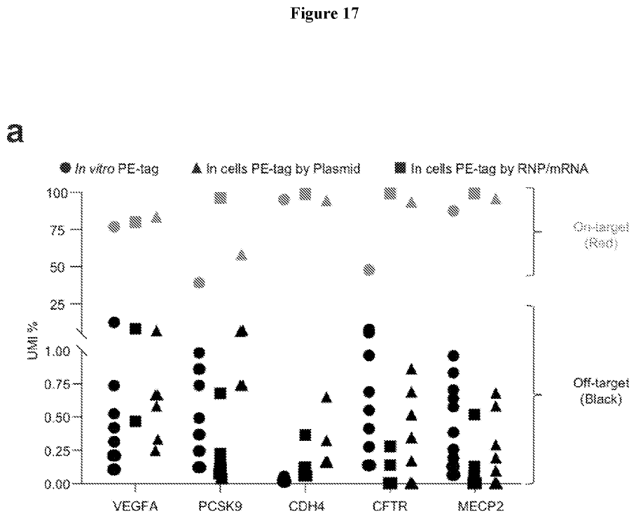

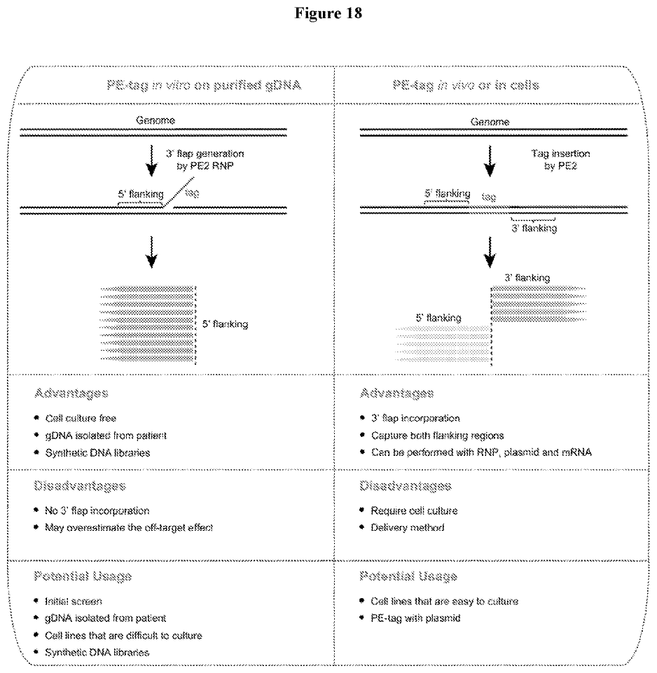

Notably, these off-target sites were also detected by GUIDE-tag analysis of SpCas9 nuclease activity delivered via RNP for CFTR and HEXA, but not MECP2. See, FIGS. 12C-E and FIG. 14A. Seventeen (17) off-target sites (e.g., six (6) CFTR sites; five (5) HEXA sites and six (6) MECP2 sites) were analyzed by amplicon deep sequencing to verify the presence of precise editing or indels. Editing was validated at eight (8) of the seventeen (17) off-target sites with an editing efficiency more than 0.1%. See, FIGS. 12F & 15B-C.

The percentage of UMI counts associated with the target site for PE-tag at almost all tested loci accounted for more than 50.0% of the total UMIs. The percentage of target site UMI counts reached 99.3% when PE-tag components were delivered as RNP or mRNA, which is probably due to the shorter half life of the prime editor. The highest percentage of UMIs at any off-target site is 24.7% at HEK4. The percentage of UMIs at most of the potential off-target sites ranges from 0.1% to 1% See, FIG. 12G.

Together, these data suggest that PE-tag enables detection of off-target gene editing induced by PE with therapeutic pegRNAs used for correction of genetic variants associated with human diseases.

### H. Utility of PE-Tag for Off-Target Analysis

The use of an in vitro PE-tag analysis enables genome-wide quantification of active prime editor recognition sites (on-target and off-target sites) in purified gDNA, which offers good sensitivity and reproducibility while avoiding the disadvantages of cell culture. For example, an in vitro PE-tag analysis can be used as an initial screen to discover potential off-target sites for a particular prime editor programmed with a specific pegRNA. These sites can then be evaluated by targeted deep sequencing with different pegRNAs to define the pegRNA sequence composition that minimizes off-target editing. This analysis provides required data for providing a foundation for clinical research.

In principle, this approach can be used on gDNA samples from perspective patients to detect potential off-target site that are associated with sequence variation in the human genome. It should also be possible to adapt this approach to fully synthetic template systems, such as those described in ONE-seq that allow the consideration of a large degree of sequence human variation. biorxiv.org/content/10.1101/2021.04.05.438458v1.

Although it is not necessary to understand the mechanism of an invention, it is believed that a prime editor protein, itself, is not required for an in vitro off-target analysis. Instead, a Cas9 H840A nickase and a MMLV reverse transcriptase can be provided as separate proteins, where the MMLV reverse transcriptase would act in trans to introduce the amplification tag element into the genome. Likewise, the pegRNA can potentially be split into an sgRNA and PBS-RTT region where the latter is used as an in trans template for off-target analysis.

PE-tag technology also provides a method to assess the alterations to a prime editing platform on a genome-wide scale. PE-tag may also play a role in improvement of specificity of prime editors. For example, as presented herein, PE-tag was used to identify potential off-target sites that are induced by epegRNAs. PE-tag may also be adapted to evaluate genome-wide specificities of prime editing when dual pegRNAs or nicking sgRNAs are included. In addition, PE-tag may also be used to assess the specificities of alternative prime editors, such as PE4, PE5, PEmax and previously reported SaPE2* or SaKKHPE2*. Liu, P. et al. Nat Commun 12, 2121 (2021). It is also believed that a PE-tag off-target analysis approach can be adapted to other types of genome editing systems that use a template in the context of a reverse transcriptase or a polymerase to introduce sequence modifications into the genome.

### I. PE-Tag Optimization Conditions

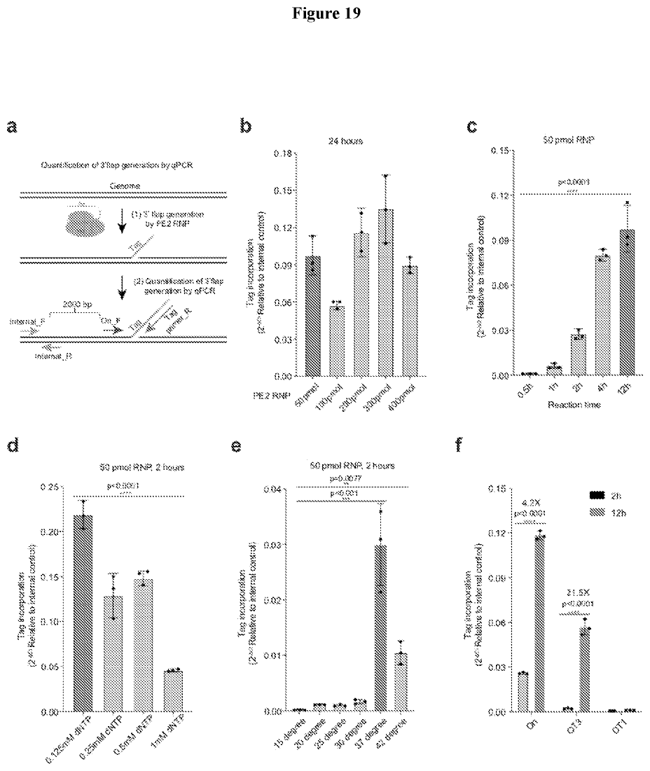

To characterize the optimal biochemical conditions for in vitro PE-tag, the efficiency of 3′ flap generation was tested under different reaction conditions quantified by qRT-PCR. These experiments defined efficient conditions for performing PE-tag in vitro on isolated gDNA. To define the optimal biochemical conditions for in vitro PE-tag, the efficiency of 3′ flap generation was tested under different conditions (PE2 RNP and dNTP concentration, reaction time and temperature. Tag incorporation rates were quantified using qRT-PCR as described above. FIG. 19.

### J. Primer Binding Site Mismatches

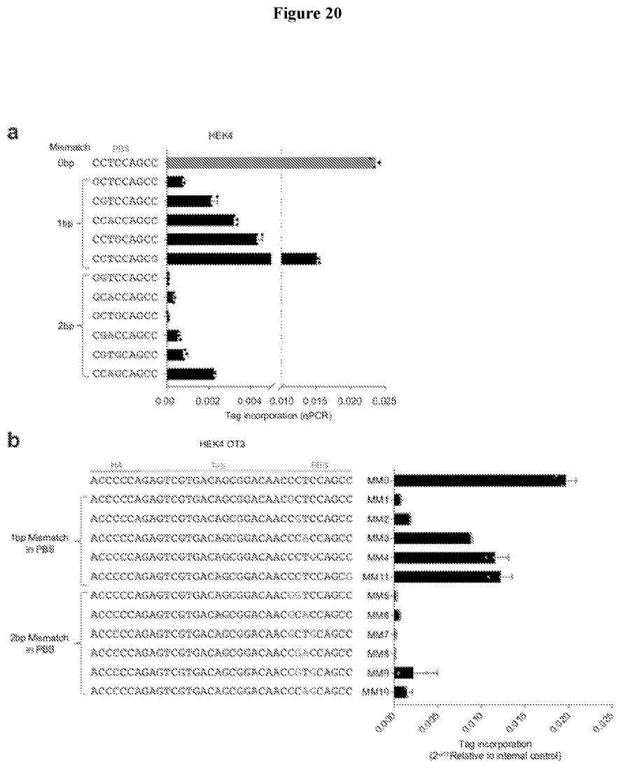

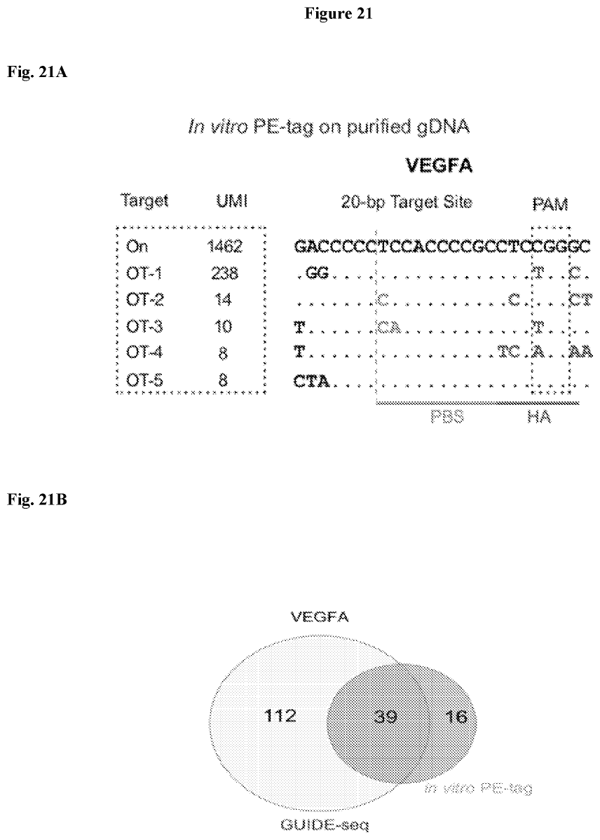

To characterize the sensitivity of PE-tag to sites containing near cognate PBS sequences, the efficiency of 3′ flap generation was tested with a series of pegRNAs which contain mismatches in the PBS region. Single mismatches in the 5′ end of PBS dramatically decreased the efficiency of 3′ flap generation, whereas mismatches in the 3′ end of PBS region only modestly affected 3′ flap generation at the target site or an off-target site. FIG. 20A and FIG. 20B. Two mismatches in the 5′ end of PBS region substantially decreased the efficiency of 3′ flap generation. Overall, these data demonstrate that PE-tag can capture PE activity at off-target sequences with substantial mismatches to the PBS sequence.

### III. Kits

In another embodiment, the present invention contemplates kits for the practice of the methods of this invention. The kits preferably include one or more containers containing: 1) an nCas9 protein and a reverse transcriptase; 2) pegRNA comprising an amplification tag element; 3) universal adaptors comprising UMIs, barcodes and amplification primer binding sites; 4) Tn5 transposase enzymes; 5) amplification primer sets; 6) a first solution comprising a buffer (10×) and deoxynucleotide triphosphates; and 7) a second solution comprising a Tn5 tagmentation buffer (10×). The kit can optionally include a control pegRNA for a known genomic target site with a defined off-target spectrum. The kit can optionally include a primer set to amplify a control genomic region residing between the amplification tag element and the associated PAM distal side of a target site. The kit can optionally include enzymes capable of performing PCR (i.e., for example, DNA polymerase, Taq polymerase and/or restriction enzymes). The kit can optionally include a pharmaceutically acceptable excipient and/or a delivery vehicle (e.g., a liposome). The reagents may be provided suspended in the excipient and/or delivery vehicle or may be provided as a separate component which can be later combined with the excipient and/or delivery vehicle. The kit may optionally contain additional therapeutics. The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for: 1) the use of the reagents in the performance of PE-tag and the generation of sequencing libraries; 2) design parameters for custom pegRNA construction for tagging on-target and off-target sites for any desired locus; 3) design parameters to construct locus-specific primers to evaluate tagging efficiency; and 4) the utilization of PE-tag sequence analysis software. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXPERIMENTAL

### Example I

### Plasmid Generation

To generate pegRNA expression plasmids, gblocks or PCR products including spacer sequences, scaffold sequences and 3′ extension sequences were amplified with indicated primers. Table 1.

TABLE 1
Sequences of primers used for locus specific
PCR amplification and Illumina sequencing.

| Description | Sequence |
|---|---|
| 4908_HEK4_ON_5p | ctacacgacgctcttccgatctCGAGGCAGAGGGTCCAAAGC |
| 4909_HEK4_ON_3p | agacgtgtgctcttccgatctCTTTCAACCCGAACGGAGACAC |
| HEK4-OT1-DS-5P | ctacacgacgctcttccgatctGGGCTCACTGCTCTCCAGAGTG |
| HEK4-OT1-DS-3P | agacgtgtgctcttccgatctGATGATTCTCCTACTTCCTCCTCGG |
| HEK4-OT4-DS-5P | ctacacgacgctcttccgatctAGTCTCACATTTTTGTCCCAGAACTTATT |
| HEK4-OT4-DS-3P | agacgtgtgctcttccgatctTTTTGTATTTTTAGTAGGGACGGGGT |
| HEK4-OT3-DS-5P | ctacacgacgctcttccgatctGAAAAGAGAAAAGCCAACGGGTTC |
| HEK4-OT3-DS-3P | agacgtgtgctcttccgatctCTGAAGGAGCACTTGGAAGATGGT |
| HEK4-OT15-DS-5P | ctacacgacgctcttccgatctGAAAGCAGAGGCTCAGAGGAAACC |
| HEK4-OT15-DS-3P | agacgtgtgctcttccgatctGCAGAGCAAACTCAAACCAGAAAATG |
| HEK4-OT10-DS-5P | ctacacgacgctcttccgatctCGCCATTTTCAAACTGCTCTAGC |
| HEK4-OT10-DS-3P | agacgtgtgctcttccgatctTGTTGACGGGAAGGGGGTGT |
| HEK4-OT13-DS-5P | ctacacgacgctcttccgatctGAGATTATATCCCGCACATGGCT |
| HEK4-OT13-DS-3P | agacgtgtgctcttccgatctGGTGGAGTCTACAGAGGCAGTCAG |
| HEK4-OT34-DS-5P | ctacacgacgctcttccgatctTCCACGGGAGATGGCTTATGTC |
| HEK4-OT34-DS-3P | agacgtgtgctcttccgatctCTGGGTTCTTAAACACTAGGCTACTCC |
| HEK4-OT7-DS-5P | ctacacgacgctcttccgatctACTTGAACCTCACGCTGTGGGC |
| HEK4-OT7-DS-3P | agacgtgtgctcttccgatctTGCTCCCCAAGGGCGTCC |
| HEK4-PCR-5P | GGCGGCTTCTCCCTCAGTCA |
| HEK4-PCR-3P | CTTTCAACCCGAACGGAGACAC |
| HEK4-ON-PCR-gib-5P | tcgacggtatcgataagcttgatGGCGGCTTCTCCCTCAGTCA |
| HEK4-OT3-PCR-gib-5P | tcgacggtatcgataagcttgatCCCACCCTCTACCCTCCCTGAT |
| HEK4-OT1-PCR-5P | CAGGACAGGCAGCCACAAGG |
| HEK4-OT1-PCR-3P | GATGATTCTCCTACTTCCTCCTCGG |
| 4938_i5_primer | AATGATACGGCGACCACCGAGATCTACAC |
| Tag primer_F | GTGACTGGAGTTCAGACGTGTGCTCTTCCGATCTGTTGTCCGCTGTCACGA |
| | CTC |
| Tag primer_R | GTGACTGGAGTTCAGACGTGTGCTCTTCCGATCTGAGTCGTGACAGCGGAC |
| | AAC |
| CFTR508-DS-5p | ctacacgacgctcttccgatctGATTATGGGAGAACTGGAGCCT |
| CFTR508-DS-3p | agacgtgtgctcttccgatctTATATGTAGACTAACCGATTGAATATGG |
| HEXA-DS-5p | ctacacgacgctcttccgatctGGCTCCGTAACTTTCTATCCGTCC |
| HEXA-DS-3p | agacgtgtgctcttccgatctGAGACAAGCAGACAGACGGGTGAG |
| MECP2T158M-PCR-5p | ACCACCATCCGCTCTGCCCTAT |
| MECP2T158M-PCR-3p | CGTGGTGCCGCTCCCTTTG |
| CFTR508-DS-5p | ctacacgacgctcttccgatctGATTATGGGAGAACTGGAGCCT |
| CFTR508-DS-3p | agacgtgtgctcttccgatctTATATGTAGACTAACCGATTGAATATGG |
| MECP2T158M-nest-5p | TGACATTGCTATGGAGAGCCTCTA |
| MECP2T158M-DS-5p | CTACACGACGCTCTTCCGATCTGCTCTAAAGTGGAGTTGATTGCG |
| MECP2T158M-DS-3p | AGACGTGTGCTCTTCCGATCTCCCCCTGGCGAAGTTTGA |
| MECP2T158M-pegRNA-5p | GTGGAAAGGACGAAACACCGCCCTAATGATTTTGACTTCAGTTTTAGAGCT |
| | AGAAATAGC |
| MECP2T158M- | CGATAAGCTTGATATCGAATTCAAAAAATAATGATTTTGACTTCACCGTAA |
| correction_pegRNA-3p | CTGGGAGGACCGACTCGGTCCCACTTTTTC |
| MECP2T158M-20 bp- | GATAAGCTTGATATCGAATTCAAAAAATAATGATTTTGACTGTTGTCCGCT |
| tag-pegRNA-3p | GTCACGACTCTCATGGTAACTGGGAGGACCGACTCGGTCCCACTTTT |
| pegCFTR_F | TGTGGAAAGGACGAAACACCGaccattaaagaaaatatcatgttttagagc |
| | tagaaatagc |
| pegCFTR_correction_R | CGATAAGCTTGATATCGAATTCAAAAAAAttaaagaaaatatCATCTTTGG |
| | TGTTTggaccgactcggtcccactt |
| pegCFTR_tag_R | CGATAAGCTTGATATCGAATTCAAAAAAAttaaagaaaatatGTTGTCCGC |
| | TGTCACGACTCcatcGGTggaccgactcggtcccactt |
| pegHEXA F | TGTGGAAAGGACGAAACACCGATCCTTCCAGTCAGGGCCATgttttagagc |
| | tagaaatagc |
| pegHEXA_correction_R | CGATAAGCTTGATATCGAATTCAAAAAAAcAGTCAGGGCCATAGGATATAC |
| | GGTTCAGGTggaccgactcggtcccactt |
| pegHEXA_Tag_R | CGATAAGCTTGATATCGAATTCAAAAAAAcAGTCAGGGCGTTGTCCGCTGT |
| | CACGACTCCATAGgaggaccgactcggtcccactt |
| CFTR-OT1-DS-5p | ctacacgacgctcttccgatctAGCCTTCAACCTGTATGCCACTG |
| CFTR-OT1-DS-3p | agacgtgtgctcttccgatctGCACTCTCGCTCAGATTCTTCCC |
| CFTR-OT2-DS-5p | ctacacgacgctcttccgatctAGTGTTTACTTGTCCTATGGCTCTCT |
| CFTR-OT2-DS-3p | agacgtgtgctcttccgatctAATTGACTTTCAGGTTGTCATTTCC |
| CFTR-OT3-DS-5p | ctacacgacgctcttccgatctAGGAGTCGCCTAACCCTAGAGAAG |
| CFTR-OT3-DS-3p | agacgtgtgctcttccgatctCTCCCCAGGTCCCCTCAAA |
| CFTR-OT4-DS-5p | ctacacgacgctcttccgatctATCCTTTGCTTGTCAGTGATACTAC |
| CFTR-OT4-DS-3p | agacgtgtgctcttccgatctTGTCTCTTCAACATACTTGTTTTCTC |
| CFTR-OT5-DS-5p | ctacacgacgctcttccgatctTATAGCATAGCAGGATAGCATTCTA |
| CFTR-OT5-DS-3p | agacgtgtgctcttccgatctTGATATAATGTCAATTTAGAGTATAGGACT |
| CFTR-OT6-DS-5p | ctacacgacgctcttccgatctGTTTTCAGAGAATATGATTGTTGG |
| CFTR-OT6-DS-3p | agacgtgtgctcttccgatctCTTCACTTTACCACCACATTTCTA |
| HEXA-OT1-DS-5p | ctacacgacgctcttccgatctTTGCTCTCTCTTCCTGTCTCCCT |
| HEXA-OT1-DS-3p | agacgtgtgctcttccgatctCCATCAGGCATCAAGAACAAGG |
| HEXA-OT2-DS-5p | ctacacgacgctcttccgatctCGGCACCCAAAAGGAGGAT |
| HEXA-OT2-DS-3p | agacgtgtgctcttccgatctCAGGGTTCAGGGGCTTCAGAG |
| HEXA-OT3-DS-5p | ctacacgacgctcttccgatctGCCCACAGCCAAGACAGAACA |
| HEXA-OT3-DS-3p | agacgtgtgctcttccgatctCACCTTGTAATGTGCTCTCAGGAAAC |
| HEXA-OT4-DS-5p | ctacacgacgctcttccgatctATGCTCGCCAGCCTGCCAC |
| HEXA-OT4-DS-3p | agacgtgtgctcttccgatctCCTGTCTCCTCTCATCCCTGTTCC |
| HEXA-OT5-DS-5p | ctacacgacgctcttccgatctGAGGTGTGAGATGCTCGCCAG |
| HEXA-OT5-DS-3p | agacgtgtgctcttccgatctGCAAGCCAGGTGACCAGCAT |
| MECP2T158M-OT1-DS-5p | ctacacgacgctcttccgatctGCTACAACTGACCTCTGTGCCC |
| MECP2T158M-OT1-DS-3p | agacgtgtgctcttccgatctCTGGTTGGAATGCTGAAGTTGTC |
| MECP2T158M-OT2-DS-5p | ctacacgacgctcttccgatctCCCAACCCTGGAGTTTCTGATTC |
| MECP2T158M-OT2-DS-3p | agacgtgtgctcttccgatctCCACACTGAGAAGGAGGTGATGAGA |
| MECP2T158M-OT3-DS-5p | ctacacgacgctcttccgatctCCAACTGCCTTGATTTGTGCC |
| MECP2T158M-OT3-DS-3p | agacgtgtgctcttccgatctTGCTTGATTCTCCTTTATAGAATTGTTACA |
| MECP2T158M-OT4-DS-5p | ctacacgacgctcttccgatctCACATTTATTTAGGTGATGCTGCT |
| MECP2T158M-OT4-DS-3p | agacgtgtgctcttccgatctAGAAAAATGTGGGATGATTGTAAAG |
| MECP2T158M-OT5-DS-5p | ctacacgacgctcttccgatctTTAGAAGATAGGAGGGCTGGAACG |
| MECP2T158M-OT5-DS-3p | agacgtgtgctcttccgatctGGCAAGGGCTTTTTCCACCA |
| MECP2T158M-OT6-DS-5p | ctacacgacgctcttccgatctGCATGGCAAAATGGCAATACAT |
| MECP2T158M-OT6-DS-3p | agacgtgtgctcttccgatctTGATGGGTGTGCTGGGATGT |

Adapter oligo sequences used to anneal and complexing with the Tn5 (Integrated DNA Technologies (IDT)).
Blue: i5 Barcode sequence
Red: hyperactive mosaic end for Tn5 transposase recognition

| | | i5 | i5 Barcode |
|---|---|---|---|
| Oligo | | Barcode | READ (Nx10 for |
| Name | Sequence 5′-3′ | Sequence | UMI bases) |
| Tn5-A | [Phos]CTGTCTCTTATACA[ddC] | | |
| bottom | | | |
| i5_N501_ | AATGATACGGCGACCACCGAGATCTACACTAGA | TAGATCGC | TAGATCGCNNNNNNNNNN |
| UMI | TCGCNNNNNNNNNNTCGTCGGCAGCGTC [symbol] | | |
| i5_N502_ | AATGATACGGCGACCACCGAGATCTACACCTCT | CTCTCTAT | CTCTCTATNNNNNNNNNN |
| UMI | CTATNNNNNNNNNNTCGTCGGCAGCGTC [symbol] | | |
| i5_N503_ | AATGATACGGCGACCACCGAGATCTACACTATC | TATCCTCT | TATCCTCTNNNNNNNNNN |
| UMI | CTCTNNNNNNNNNNTCGTCGGCAGCGTC [symbol] | | |
| i5_N504_ | AATGATACGGCGACCACCGAGATCTACACAGAG | AGAGTAGA | AGAGTAGANNNNNNNNNN |
| UMI | TAGANNNNNNNNNNTCGTCGGCAGCGTC [symbol] | | |
| i5_N505_ | AATGATACGGCGACCACCGAGATCTACACGTAA | GTAAGGAG | GTAAGGAGNNNNNNNNNN |
| UMI | GGAGNNNNNNNNNNTCGTCGGCAGCGTC [symbol] | | |
| i5_N506_ | AATGATACGGCGACCACCGAGATCTACACACTG | ACTGCATA | ACTGCATANNNNNNNNNN |
| UMI | CATANNNNNNNNNNTCGTCGGCAGCGTC [symbol] | | |
| i5_N507_ | AATGATACGGCGACCACCGAGATCTACACAAGG | AAGGAGTA | AAGGAGTANNNNNNNNNN |
| UMI | AGTANNNNNNNNNNTCGTCGGCAGCGTC [symbol] | | |
| i5_N508_ | AATGATACGGCGACCACCGAGATCTACACCTAA | CTAAGCCT | CTAAGCCTNNNNNNNNNN |
| UMI | GCCTNNNNNNNNNNTCGTCGGCAGCGTC [symbol] | | |

[symbol]

indicates data missing or illegible when filed

i5 oligo sequence for Round 1 and 2 PCR:

| Oligo Name | Sequence 5′-3′ |
|---|---|
| i5 | AATGATACGGCGACCACCGAGATCTACAC |

i7 Barcoded oligo sequences for Round 2 PCR:

| Oligo | | i7 Barcode | i7 BC |
|---|---|---|---|
| Name | Sequence (5′-3′) | Sequence | READ |
| i7_N701 | CAAGCAGAAGACGGCATACGAGATAGCGGAATGTGACTGGAGTTCAGACGTGT | AGCGGAAT | ATTCCGCT |
| i7_N702 | CAAGCAGAAGACGGCATACGAGATGATCATGCGTGACTGGAGTTCAGACGTGT | GATCATGC | GCATGATG |
| i7_N703 | CAAGCAGAAGACGGCATACGAGATAAGACGGAGTGACTGGAGTTCAGACGTGT | AAGACGGA | TCCGTCTT |
| i7_N704 | CAAGCAGAAGACGGCATACGAGATCGAGTCCTGTGACTGGAGTTCAGACGTGT | CGAGTCCT | AGGACTCG |
| i7_N705 | CAAGCAGAAGACGGCATACGAGATTCCTCAGGGTGACTGGAGTTCAGACGTGT | TCCTCAGG | CCTGAGGA |
| i7_N706 | CAAGCAGAAGACGGCATACGAGATGTACGGATGTGACTGGAGTTCAGACGTGT | GTACGGAT | ATCCGTAC |
| i7_N707 | CAAGCAGAAGACGGCATACGAGATCATCTCTCGTGACTGGAGTTCAGACGTGT | CATCTCTC | GAGAGATG |
| i7_N710 | CAAGCAGAAGACGGCATACGAGATGTCGGAGCGTGACTGGAGTTCAGACGTGT | GTCGGAGC | GCTCCGAC |
| i7_N711 | CAAGCAGAAGACGGCATACGAGATACGGAGAAGTGACTGGAGTTCAGACGTGT | ACGGAGAA | TTCTCCGT |
| i7_N712 | CAAGCAGAAGACGGCATACGAGATAGGAGATGGTGACTGGAGTTCAGACGTGT | AGGAGATG | CATCTCCT |
| i7_N714 | CAAGCAGAAGACGGCATACGAGATAGTACTCGGTGACTGGAGTTCAGACGTGT | AGTACTCG | CGAGTACT |
| i7_N715 | CAAGCAGAAGACGGCATACGAGATGGACTCTAGTGACTGGAGTTCAGACGTGT | GGACTCTA | TAGAGTCC |

Using Phusion master mix (ThermoFisher Scientifc), which were subsequently cloned into a custom vector, BfuAI and EcoR I digested) by the Gibson assembly method (NEB). To generate sgRNA expression plasmids, annealed oligos were cloned into BfuAI-digested vector. See, SEQ ID NO: 2; and Table 2.

SEQ ID NO: 2: Sequence of backbone plasmid used for pegRNA and
nicking sgRNA cloning Backbone of pegRNA used for PE2: U6
promoter + SpCas9-sgRNA scaffold
GTGGCACTTTTCGGGGAAATGTGCGCGGAACCCCTATTTGTTTATTTTTCTAAATACATTCAAA
TATGTATCCGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCCTGTTT
TTGCTCACCCAGAAACGCTGGTGAAAGTAAAAGATGCTGAAGATCAGTTGGGTGCACGAGTGGG
TTACATCGAACTGGATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCGTATTGACGCCGGGC
AAGAGCAACTCGGTCGCCGCATACACTATTCTCAGAATGACTTGGTTGAGTACTCACCAGTCAC
AGAAAAGCATCTTACGGATGGCATGACAGTAAGAGAATTATGCAGTGCTGCCATAACCATGAGT
GATAACACTGCGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCGCAAACTATTA
ACTGGCGAACTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAAATCTGGAGC
CGGTGAGCGTGGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAGATCGCTGAGA
TAGGTGCCTCACTGATTAAGCATTGGTAACTGTCAGACCAAGTTTACTCATATATACTTTAGAT
TGATTTAAAACTTCATTTTTAATTTAAAAGGATCTAGGTGAAGATCCTTTTTGATAATCTCATG
ACCAAAATCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTAACTGGCTTC
AGCAGAGCGCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCC
TTTGAGTGAGCTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTCATTAATGCAG
CTGGCACGACAGGTTTCCCGACTGGAAAGCGGGCAGTGAGCGCAACGCAATTAATGTGAGTTAG
CTCACTCATTAGGCACCCCAGGCTTTACACTTTATGCTTCCGGCTCGTATGTTGTGTGGAATTG
TGAGCGGATAACAATTTCACACAGGAAACAGCTATGACCATGATTACGCCAAGCGCGCAATTAA
CCCTCACTAAAGGGAACAAAAGCTGGAGCTCCACCGCGGTGGCGGCCGCCCCCTTCACCGAGGG
CCTATTTCCCATGATTCCTTCATATTTGCATATACGATACAAGGCTGTTAGAGAGATAATTGGA
ATTAATTTGACTGTAAACACAAAGATATTAGTACAAAATACGTGACGTAGAAAGTAATAATTTC
TTGGGTAGTTTGCAGTTTTAAAATTATGTTTTAAAATGGACTATCATATGCTTACCGTAACTTG
AAAGTATTTCGATTTCTTGGCTTTATATATCTTGTGGAAAGGACGAAACACCGGTGTGCAGGTG
AGTGATCCAAACGCCCGGCGGCAACCGAGCGTTCTGAACAAATCCAGATGGAGTTCTGAGGTCA
TTACTGGATCTATCAACAGGAGTCCAAGCGAGCTCTCGAACCCCAGAGTCCCGCTCAGAAGAAC
TCGTCAAGAAGGCGATAGAAGGCGATGCGCTGCGAATCGGGAGCGGCGATACCGTAAAGCACGA
GGAAGCGGTCAGCCCATTCGCCGCCAAGCTCTTCAGCAATATCACGGGTAGCCAACGCTATGTC
CTGATAGCGGTCCGCCACACCCAGCCGGCCACAGTCGATGAATCCAGAAAAGCGGCCATTTTCC
ACCATGATATTCGGCAAGCAGGCATCGCCATGGGTCACGACGAGATCCTCGCCGTCGGGCATGC
GCGCCTTGAGCCTGGCGAACAGTTCGGCTGGCGCGAGCCCCTGATGCTCTTCGTCCAGATCATC
CTGATCGACAAGACCGGCTTCCATCCGAGTACGTGCTCGCTCGATGCGATGTTTCGCTTGGTGG
TCGAATGGGCAGGTAGCCGGATCAAGCGTATGCAGCCGCCGCATTGCATCAGCCATGATGGATA
CTTTCTCGGCAGGAGCAAGGTGAGATGACAGGAGATCCTGCCCCGGCACTTCGCCCAATAGCAG
CCAGTCCCTTCCCGCTTCAGTGACAACGTCGAGCACAGCTGCGCAAGGAACGCCCGTCGTGGCC
AGCCACGATAGCCGCGCTGCCTCGTCCTGCAGTTCATTCAGGGCACCGGACAGGTCGGTCTTGA
CAAAAAGAACCGGGCGCCCCTGCGCTGACAGCCGGAACACGGCGGCATCAGAGCAGCCGATTGT
CTGTTGTGCCCAGTCATAGCCGAATAGCCTCTCCACCCAAGCGGCCGGAGAACCTGCGTGCAAT
CCATCTTGTTCAATCATGCGAAACGATCCTCATCCTGTCTCTTGATCAGATCTTGATCCCCTGC
GCCATCAGATCCTTGGCGGCAAGAAAGCCATCCAGTTTACTTTGCAGGGCTTCCCAACCTTACC
AGAGGGCGCCCCAGCTGGCAATTCCGACGGATCAAGAACCTGCTGACGTTTTAGAGCTAGAAATAG
CAAGTTAAAATAAGGCTAGTCCGTTATCAACTTGAAAAAGTGGGACCGAGTCGGTCCTTTTTTTGAA
TTCGATATCAAGCTTATCGATACCGTCGACCTCGAGGGGGGGCCCGGTACCCAATTCGCCCTATAG
TGAGTCGTATTACGCGCGCTCACTGGCCGTCGTTTTACAACGTCGTGACTGGGAAAACCCTGGC
GTTACCCAACTTAATCGCCTTGCAGCACATCCCCCTTTCGCCAGCTGGCGTAATAGCGAAGAGG
CCCGCACCGATCGCCCTTCCCAACAGTTGCGCAGCCTGAATGGCGAATGGGACGCGCCCTGTAG
CGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGACCCCAA
AAAACTTGATTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACC
CTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAG
pU6-HEK4_pegRNA_20-7: U6 promoter + Spacer + sgRNA scaffold +
RTT + PBS
GAGGGCCTATTTCCCATGATTCCTTCATATTTGCATATACGATACAAGGCTGTTAGA
GAGATAATTGGAATTAATTTGACTGTAAACACAAAGATATTAGTACAAAATACGTGA
CGTAGAAAGTAATAATTTCTTGGGTAGTTTGCAGTTTTAAAATTATGTTTTAAAATGG
ACTATCATATGCTTACCGTAACTTGAAAGTATTTCGATTTCTTGGCTTTATATATCTT
GTGGAAAGGACGAAACACCGGCACTGCGGCTGGAGGTGGgttttagagctagaaatag
caagttaaaataaggctagtccgttatcaacttgaaaaagtgggaccgagtcggtccA
CCCCCAGAGTCGTGACAGCGGACAACCCTCCAGCCTTTTTTT
sgRNA scaffold sequence-spacer and 3′ extensions are appended
to this core sequence:
GTTTTAGAGCTAGAAATAGCAAGTTAAAATAAGGCTAGTCCGTTATCAACTTGAAAA
AGTGGGACCGAGTCGGTCC

TABLE 2
Representative sequences of pegRNAs and sgRNAs shown in 5′ to 3′ orientation.

| | spacer sequence | |
|---|---|---|
| pegRNA | (5′-3′) | 3′ extension |
| HEK4 7-7 | GGCACTGCGGCTGG | ACCCCCAGAGTCGTCCTCCAGCC |
| | AGGTGG | |
| HEK4 7-16 | GGCACTGCGGCTGG | TCCGCTTTAACCCCCAGAGTCGTCCTCCAGCC |
| | AGGTGG | |
| HEK4 7-20 | GGCACTGCGGCTGG | AGTCTCCGCTTTAACCCCCAGAGTCGTCCTCCAGCC |
| | AGGTGG | |
| HEK4 11-7 | GGCACTGCGGCTGG | ACCCCCAGAGTCGTGACACCTCCAGCC |
| | AGGTGG | |
| HEK4 11-16 | GGCACTGCGGCTGG | TCCGCTTTAACCCCCAGAGTCGTGACACCTCCAGCC |
| | AGGTGG | |
| HEK4 11-20 | GGCACTGCGGCTGG | AGTCTCCGCTTTAACCCCCAGAGTCGTGACACCTCCAGCC |
| | AGGTGG | |
| HEK4 20-7 | GGCACTGCGGCTGG | ACCCCCAGAGTCGTGACAGCGGACAACCCTCCAGCC |
| | AGGTGG | |
| HEK4 20-16 | GGCACTGCGGCTGG | TCCGCTTTAACCCCCAGAGTCGTGACAGCGGACAACCCTCCAGCC |
| | AGGTGG | |
| HEK4 20-20 | GGCACTGCGGCTGG | AGTCTCCGCTTTAACCCCCAGAGTCGTGACAGCGGACAACCCTCCAGCC |
| | AGGTGG | |
| HEK4 20-7 | GGCACTGCGGCTGG | ACCCCCAGAGTCGTGACAGCGGACAACCCTCCAGCC |
| | AGGTGG | |
| VEGFA | GACCCCCTCCACCC | GCCCGGAGGAGTCGTGACAGCGGACAACGCGGGGTGGA |
| 20-8 | CGCCTC | |
| HEK4 peg | GGCACTGCGGCTGG | ACCCCCAGAGTCGTGACAGCGGACAACCCTCCAGCC |
| 20-7 | AGGTGG | |
| HEK4 epeg | GGCACTGCGGCTGG | ACCCCCAGAGTCGTGACAGCGGACAACCCTCCAGCCAATCTATACGCGGTTCTATCTAGTTACGCG |
| 20-7 | AGGTGG | TTAAACCAACTAGAA |
| PCSK9 peg | GAGCAGCAGCGGCG | TGCCGCTGGAGTCGTGACAGCGGACAACTTGCCGCCGC |
| 20-8 | GCAACAG | |
| PCSK9 epeg | GAGCAGCAGCGGCG | TGCCGCTGGAGTCGTGACAGCGGACAACTTGCCGCCGCTTATATACCGCGGTTCTATCTAGTTACG |
| 20-8 | GCAACAG | CGTTAAACCAACTAGAA |
| CFTR peg | Gaccattaaagaaa | AAACACCAAAGATGatattttctttaa |
| Correction | atatcat | |
| CFTR epeg | Gaccattaaagaaa | AAACACCAAAGATGatattttctttaaACACTCTGCGCGGTTCTATCTAGTTACGCGTTAAACCAA |
| Correction | atatcat | CTAGAA |
| HEXA peg | GATCCTTCCAGTCA | ACCTGAACCGTATATCCTATGGCCCTGACTG |
| Correction | GGGCCAT | |
| HEXA epeg | GATCCTTCCAGTCA | ACCTGAACCGTATATCCTATGGCCCTGACTGTCCCTATACGCGGTTCTATCTAGTTACGCGTTAAA |
| Correction | GGGCCAT | CCAACTAGAA |
| MECP2 peg | GCCCTAATGATTTT | TCCCAGTTACggTGAAGTCAAAATCATTA |
| Correction | GACTTCA | |
| MECP2 epeg | GCCCTAATGATTTT | TCCCAGTTACggTGAAGTCAAAATCATTAATCAGTTACGCGGTTCTATCTAGTTACGCGTTAAACC |
| Correction | GACTTCA | AACTAGAA |
| CFTR peg | Gaccattaaagaaa | ACCgatgGAGTCGTGACAGCGGACAACatattttctttaa |
| 20-7 | atatcat | |
| CFTR epeg | Gaccattaaagaaa | ACCgatgGAGTCGTGACAGCGGACAACatattttctttaaCCCAAACCCGCGGTTCTATCTAGTTA |
| 20-7 | atatcat | CGCGTTAAACCAACTAGAA |
| HEXA peg | GATCCTTCCAGTCA | TCCTATGGAGTCGTGACAGCGGACAACGCCCTGACTG |
| 20-7 | GGGCCAT | |
| HEXA epeg | GATCCTTCCAGTCA | TCCTATGGAGTCGTGACAGCGGACAACGCCCTGACTGCCTACAAGCGCGGTTCTATCTAGTTACGC |
| 20-7 | GGGCCAT | GTTAAACCAACTAGAA |
| MECP2 peg | GCCCTAATGATTTT | TCCCAGTTACCATGAGAGTCGTGACAGCGGACAACAGTCAAAATCATTA |
| 20-10 | GACTTCA | |
| MECP2 epeg | GCCCTAATGATTTT | TCCCAGTTACCATGAGAGTCGTGACAGCGGACAACAGTCAAAATCATTATTATATCCCGCGGTTCT |
| 20-10 | GACTTCA | ATCTAGTTACGCGTTAAACCAACTAGAA |
| HEK4 G-T-7 | GGCACTGCGGCTGG | TAACCCCAACCTCCAGCC |
| | AGGTGG | |
| HEK4 G-T-16 | GGCACTGCGGCTGG | TCTCCGCTTTAACCCCAACCTCCAGCC |
| | AGGTGG | |
| HEK4 G-T-20 | GGCACTGCGGCTGG | AGAGTCTCCGCTTTAACCCCAACCTCCAGCC |
| | AGGTGG | |
| HEK4 1-Del-7 | GGCACTGCGGCTGG | AACCCCCCCTCCAGCC |
| | AGGTGG | |
| HEK4 1-Del-16 | GGCACTGCGGCTGG | TCTCCGCTTTAACCCCCCCTCCAGCC |
| | AGGTGG | |
| HEK4 1-Del-20 | GGCACTGCGGCTGG | AGAGTCTCCGCTTTAACCCCCCCTCCAGCC |
| | AGGTGG | |
| HEK4 1-Inser- | GGCACTGCGGCTGG | AACCCCCAACCTCCAGCC |
| 7 | AGGTGG | |
| HEK4 1-Inser- | GGCACTGCGGCTGG | TCTCCGCTTTAACCCCCAACCTCCAGCC |
| 16 | AGGTGG | |
| HEK4 1-Inser- | GGCACTGCGGCTGG | AGAGTCTCCGCTTTAACCCCCAACCTCCAGCC |
| 20 | AGGTGG | |
| CDH4 20-10 | GAGTCCGAGCAGAA | TCCTCCACCTGAGTCGTGACAGCGGACAACCCTCCGGCCGCA |
| | GAAGAA | |
| CFTR-sgRNA | GACCATTAAAGAAA | |
| | ATATCAT | |
| HEXA-sgRNA | GATCCTTCCAGTCA | |
| | GGGCCAT | |
| MECP2T158M- | GCCCTAATGATTTT | |
| sgRNA | GACTTCA | |

Sequences of Synthetic pegRNAs from IDT

| pegRNA | spacer sequence (5′-3′) |
|---|---|
| CDH4-PE-tag | /AlTR1/rUrGrCrArCrUrGrCrGrGrCrCrGrGrArGrGrArGrGrGrUrUrUrUrArGrArGrCrUrArGrArArArUr |
| | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrUrUrArCrUrUrUrCrCrArUrCrCrUrCrCrArCrCrUrGrArGr |
| | UrCrGrUrGrArCrArGrCrGrGrArCrArArCrCrCrUrCrCrGrGrCrCrGrCrA/AlTR2/ |
| HEK4-PE-tag | /AlTR1/rGrGrCrArCrUrGrCrGrGrCrUrGrGrArGrGrUrGrGrGrUrUrUrUrArGrArGrCrUrArGrArArArUr |
| | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrArGrUrCrUrCrCrGrCrUrUrUrArArCrCrCrCrCrArGrArGr |
| | UrCrGrUrGrArCrArGrCrGrGrArCrArArCrCrCrUrCrCrArGrCrC/AlTR2/ |
| PCSK9-PE-tag | /AlTR1/rArGrCrArGrCrArGrCrGrGrCrGrGrCrArArCrArGrGrUrUrUrUrArGrArGrCrUrArGrArArArUr |
| | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrGrGrUrGrGrCrCrGrCrUrGrUrUrGrCrCrGrCrUrGrGrArGr |
| | UrCrGrUrGrArCrArGrCrGrGrArCrArArCrUrUrGrCrCrGrCrCrGrC/AlTR2/ |
| VEGFA-PE-tag- | /AlTR1/rGrArCrCrCrCrCrUrCrCrArCrCrCrCrGrCrCrUrCrGrUrUrUrUrArGrArGrCrUrArGrArArArUr |
| pegRNA | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrGrCrCrCrGrGrArGrGrArGrUrCrGrUrGrArCrArGrCrGrGr |
| | ArCrArArCrGrCrGrGrGrGrUrGrGrA/AlTR2/ |
| HEXA-20-Tag- | /AlTR1/rArUrCrCrUrUrCrCrArGrUrCrArGrGrGrCrCrArUrGrUrUrUrUrArGrArGrCrUrArGrArArArUr |
| pegRNA | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrUrCrCrUrArUrGrGrArGrUrCrGrUrGrArCrArGrCrGrGrAr |
| | CrArArCrGrCrCrCrUrGrArCrUrG/AlTR2/ |
| HEXA-pegRNA- | /AlTR1/rGrArUrCrCrUrUrCrCrArGrUrCrArGrGrGrCrCrArUrGrUrUrUrUrArGrArGrCrUrArGrArArAr |
| correction | UrArGrCrArArGrUrUrArArArArUrArArGrGrCrUrArGrUrCrCrGrUrUrArUrCrArArCrUrUrGrArArArAr |
| | ArGrUrGrGrGrArCrCrGrArGrUrCrGrGrUrCrCrArCrCrUrGrArArCrCrGrUrArUrArUrCrCrUrArUrGrGr |
| | CrCrCrUrGrArCrUrG/AlTR2/ |
| Rett T158M_20- | /AlTR1/rCrCrCrUrArArUrGrArUrUrUrUrGrArCrUrUrCrArGrUrUrUrUrArGrArGrCrUrArGrArArArUr |
| tag_pegRNA | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrUrCrCrCrArGrUrUrArCrCrArUrGrArGrArGrUrCrGrUrGr |
| | ArCrArGrCrGrGrArCrArArCrArGrUrCrArArArArUrCrArUrUrA/AlTR2/ |
| Rett | /AlTR1/rCrCrCrUrArArUrGrArUrUrUrUrGrArCrUrUrCrArGrUrUrUrUrArGrArGrCrUrArGrArArArUr |
| T158M_correct_ | ArGrCrArArGrUrUrArArArArUrArArGrGrCrUrArGrUrCrCrGrUrUrArUrCrArArCrUrUrGrArArArArAr |
| pegRNA | GrUrGrGrGrArCrCrGrArGrUrCrGrGrUrCrCrUrCrCrCrArGrUrUrArCrGrGrUrGrArArGrUrCrArArArAr |
| | UrCrArUrUrA/AlTR2/ |
| CFTR-20-Tag- | /AlTR1/rArCrCrArUrUrArArArGrArArArArUrArUrCrArUrGrUrUrUrUrArGrArGrCrUrArGrArArArUr |
| pegRNA | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrArCrCrGrArUrGrGrArGrUrCrGrUrGrArCrArGrCrGrGrAr |
| | CrArArCrArUrArUrUrUrUrCrUrUrUrArA/AlTR2/ |
| CFTR-pegRNA- | /AlTR1/rArCrCrArUrUrArArArGrArArArArUrArUrCrArUrGrUrUrUrUrArGrArGrCrUrArGrArArArUr |
| correction | ArGrCrArArGrUrUrArArArArUrArArGrGrCrUrArGrUrCrCrGrUrUrArUrCrArArCrUrUrGrArArArArAr |
| | GrUrGrGrGrArCrCrGrArGrUrCrGrGrUrCrCrArArArCrArCrCrArArArGrArUrGrArUrArUrUrUrUrCrUr |
| | UrUrArA/AlTR2/ |
| HEXA-sgRNA | mA*mU*mC*rCrUrUrCrCrArGrUrCrArGrGrGrCrCrArUrGrUrUrUrUrArGrArGrCrUrArGrArArArUrArGr |
| | CrArArGrUrUrArArArArUrArArGrGrCrUrArGrUrCrCrGrUrUrArUrCrArArCrUrUrGrArArArArArGrUr |
| | GrGrCrArCrCrGrArGrUrCrGrGrUrGrCmU*mU*mU*rU |
| Rett_T158M_ | mC*mC*mC*rUrArArUrGrArUrUrUrUrGrArCrUrUrCrArGrUrUrUrUrArGrArGrCrUrArGrArArArUrArGr |
| sgRNA | CrArArGrUrUrArArArArUrArArGrGrCrUrArGrUrCrCrGrUrUrArUrCrArArCrUrUrGrArArArArArGrUr |
| | GrGrCrArCrCrGrArGrUrCrGrGrUrGrCmU*mU*mU*rU |
| CFTR_sgRNA | mA*mC*mC*rArUrUrArArArGrArArArArUrArUrCrArUrGrUrUrUrUrArGrArGrCrUrArGrArArArUrArGr |
| | CrArArGrUrUrArArArArUrArArGrGrCrUrArGrUrCrCrGrUrUrArUrCrArArCrUrUrGrArArArArArGrUr |
| | GrGrCrArCrCrGrArGrUrCrGrGrUrGrCmU*mU*mU*rU |

All plasmids used for in vitro and in vivo experiments were purified using Midiprep kit including endotoxin removal step (Qiagen). pCMV-PE2 was a gift from David Liu (Addgene plasmid #132775). To generate pET-21a-PE2-His, a 6×His-tag was added to the 5′ of the PE2 ORF which were then cloned into the bacterial expression plasmid pET-21a. To generate pET-21a-MMTV-RT-His, MMTV-RT ORF was amplified from PE2 ORF which were then cloned into the bacterial expression plasmid pET-21a.

### Example II

### Cell Culture And Transfection

HEK293T cells, HEPA1-6 cells and U2OS cells were purchased from ATCC. To construct a HEK293T based cell line that is homozygous for the HEXA 4 bp duplication, we designed a donor template to knock-in the HEXA 4 bp duplication into the Sec61b locus in HEK293T cells. 20 pmols of SpyCas9 and 25 pmols of sgRNA targeting the sec61b locus were complexed to form RNPs at room temperature in a final volume of 12 ul. 200,000 cells per reaction were resuspended in 10 ul of RNP-buffer R mix and electroporated into HEK293 Ts along with 30 pmols of donor template using the ThermoFisher Neon transfection system (1150V 20 ms 2 pulses). Cells were plated into 24 well plates with pre-equilibrated 500 ul of antibiotic free culture media and grown in a humidified incubator at 37 C and 5% CO₂ for 3 days. 100 ul of electroporated cells were harvested for indel analysis and rest were further propagated to establish clones. The remaining cells were counted and appropriate amounts were plated into 96 wells such that each well contained at least one cell via serial dilutions. After 20 days of culturing, wells containing individual clones were further propagated into 24 well plates. 100 ul of the clonal population was harvested for indel analysis and rest were subjected to further propagation. Cells homozygous for the HEXA 4 bp duplication were frozen down and used in subsequent experiments. HEK293T cell with MECP2^{T158M} is a gift from Sontheimer lab. CFF-16HBEge CFTR^{Δ508} were obtained from the Cystic Fibrosis Foundation's Therapeutic Lab (Lexington, MA) and were cultured in Modified Eagle's Medium supplemented with 10% FBS at 37° and 5% CO₂. All other cells were maintained in Dulbecco's Modified Eagle's Medium supplemented with 10% FBS at 37° and 5% CO₂.

For transfection-based editing experiments, HEK293T and Hepa1-6 cells were plated 100,000 cells per well in a 48-well plate. 24 hours later, the cells were co-transfected with 540 ng of prime editor plasmid, 270 ng of pegRNA plasmid. Lipofectamine 3000 (Invitrogen) was used for the transfection according to the manufacturer's instructions. To determine editing rates at endogenous genomic loci, HEK293T cells were cultured for 3 days after transfection, and genomic DNA was isolated using QIAamp DNA mini kit (QIAGEN) according to the manufacturer's instructions.

For electropration-based editing experiments in Hepa1-6, U2OS or HEK293T cells, 25k cells were electroporated with 5 μmol of indicated PE2 RNP or 200 ng PE mRNA and 100 pmol pegRNA. 400 pmol synthetic pegRNA was incubated with 200 pmol PE protein in 15 ul PBS for 20 min at room temperature to form PE2 RNP. gDNA were isolated 3 days after electropration from each group and saved at −80 for PE-tag library preparation.

### Example III

### Animals

All animal experiments were authorized by the Institutional Animal Care and Use Committee (IACUC) at UMASS medical school. All DNA vectors were prepared by EndoFreeMaxi kit (Qiagen) and were delivered through hydrodynamic tail-vein injection. For in vivo Pcsk9 gene editing, C57BL/6J (Strain #000664) mice were purchased from Jackson Laboratories. 6 Eight-week-old mice were randomally allocated into two groups. Mice were injected with 2-2.5 ml 0.9% saline containing (i) 30 μg PE2 expression vector, or (ii) 30 μg PE2 and 15 ug 20-7 Pcsk9 pegRNA expression vectors. Animals were sacrificed at the end of each experiment (7 days for Pcsk9 editing). gDNA were isolated from fresh livers for library preparation. No animals were excluded from the analyses. No sample size calculation was performed and each group consisted of at least three mice for statistical analysis.

### Example IV

### Protein Purification

### Tn5

Tn5 purification utilized a modified protocol that includes the addition of PEI and (NH4)₂SO₄ precipitations. Pengpeng Liu et al. Nucleic Acids Res. 2019 May 7; 47(8):4169-4180. pET-45b(+)-Tn5 (for Tn5 protein, a gift from Frank Pugh-Addgene plasmid #112112) were introduced into E. coli Rosetta2(DE3)pLysS cells (EMD Millipore) for protein overexpression. Cells were grown at 37° C. to an OD600 of ~0.2, then shifted to 18° C. and, at OD600 of ~0.4, induced for 16 hours with IPTG (0.7 mM final concentration). Following induction, cells were pelleted by centrifugation and then resuspended with Nickel-NTA buffer (20 mM TRIS+1 M NaCl+20 mM imidazole+1 mM TCEP, pH 7.5) supplemented with HALT Protease Inhibitor Cocktail, EDTA-Free (100×) [ThermoFisher] and lysed with LM-20 Microfluidizer (Microfluidics) following the manufacturer's instructions. The nucleic acids were removed by precipitation with 0.25% w/v PEI and centrifuged @10,000×g for 10 min. The PEI was removed by precipitating the protein with 70% (NH4)₂SO₄ at 4° C. and centrifuged @12,000×g for 15 min. The protein pellet was purified with Ni-NTA resin and eluted with elution buffer (20 mM TRIS, 500 mM NaCl, 250 mM Imidazole, 10% w/v glycerol, pH 7.5). Tn5 protein was dialyzed overnight at 4° C. in 20 mM HEPES, 500 mM NaCl, 1 mM EDTA, 10% w/v (8% v/v) glycerol, pH 7.5. Subsequently, Tn5 protein was step dialyzed from 500 mM NaCl to 200 mM NaCl (Final dialysis buffer: 20 mM HEPES, 200 mM NaCl, 1 mM EDTA, 10% w/v glycerol, pH 7.5). Next, the Tn5 protein was purified by cation exchange chromatography (Column=5 ml HiTrap-S, Buffer A=20 mM HEPES pH 7.5+1 mM TCEP, Buffer B=20 mM HEPES pH 7.5+1 M NaCl+1 mM TCEP, Flow rate=5 ml/min, CV=column volume=5 ml). The primary protein peak from the CEC was dialyzed to 2×Tn5 buffer (100 mM HEPES-KOH at pH 7.2, 0.2M NaCl, 0.2 mM EDTA, 2 mM DTT, 0.2% Triton X-100, 20% glycerol) and concentrated in an Ultra-15 Centrifugal Filters Ultracel-30K (Amicon) to a concentration of 63.5 μM. Finally, 0.827 volumes of 100% glycerol was added for a final concentration of 55% glycerol and then the Tn5 is stored at −20° C. until needed for transposome assembly.

### PE2

PE2 Protein purification followed a previously described protocols for Cas9-based proteins. Wu, Y. et al. Nat Med 25, 776-783 (2019). pET-21a-PE2-His was introduced into E. coli Rosetta2(DE3)pLysS cells (EMD Millipore) for protein overexpression. Cells were grown at 37° C. to an OD600 of ~0.2, then shifted to 18° C. and, at OD600 of ~0.4, induced for 16 hours with IPTG (0.7 mM final concentration). Following induction, cells were pelleted by centrifugation and then resuspended with Nickel-NTA buffer (20 mM TRIS+1 M NaCl+20 mM imidazole+1 mM TCEP, pH 7.5) supplemented with HALT Protease Inhibitor Cocktail, EDTA-Free (100×) [ThermoFisher] and lysed with LM-20 Microfluidizer (Microfluidics) following the manufacturer's instructions. The protein pellet was then purified with Ni-NTA resin and eluted with elution buffer (20 mM TRIS, 500 mM NaCl, 250 mM Imidazole, 10% w/v glycerol, pH 7.5). The PE2 protein was dialyzed overnight at 4° C. in 20 mM HEPES, 500 mM NaCl, 1 mM EDTA, 10% w/v (8% v/v) glycerol, pH 7.5. Subsequently, The PE2 protein was step dialyzed from 500 mM NaCl to 200 mM NaCl (Final dialysis buffer: 20 mM HEPES, 200 mM NaCl, 1 mM EDTA, 10% w/v glycerol, pH 7.5). Next, the PE2 protein was purified by cation exchange chromatography (Column=5 ml HiTrap-S, Buffer A=20 mM HEPES pH 7.5+1 mM TCEP, Buffer B=20 mM HEPES pH 7.5+1 M NaCl+1 mM TCEP, Flow rate=5 ml/min, CV=column volume=5 ml). The primary protein peak was dialyzed into a buffer of 20 mM HEPES and 300 mM NaCl, pH 7.4 and then concentrated to ~40 mM.

### Example V

### In Vitro PE-Tag

400 pmol synthetic pegRNA was incubated with 200 pmol PE protein in 15 ul PBS for 20 min at room temperature to form PE2 RNP. Following complex formation, the PE2 RNP was mixed with the reaction buffer (10 mM dNTP, 5% glycerol, 100 mM KCl, 10 mM Hepes pH 7.5, 0.2 mM EDTA, 3 mM MgCl₂, 5 mM DTT final concentration) and 2 ug purified gDNA in a total volume of 30 ul for 8 hours at 37° C. Then the mixture was treated with 10 ul RNase A (50 ug/ml) to remove the pegRNA and the PE2 RNP treated gDNA was purified using a DNeasy Blood & Tissue Kit (Qiagen). The purified gDNA was then used for Tn5 tagmentation and library preparation.

### Example VI

### Tn5 Tagmentation and Library Preparation for PE-Tag

Adaptor oligonucleotides for assembly were synthesized by IDT. Tn5 transposome assembly was done by incubating 158 μg Tn5 with 1.4 nmol annealed oligo (contains the full-length Illumina forward (i5) adapter, a sample barcode, and unique molecule identifier (UMI)) at room temperature for 60 mins. Giannoukos, G. et al. BMC genomics 19, 212 (2018).

For genome tagmentation, 200 ng of genomic DNA was incubated with 2 ul of assembled transposome at 55° C. for 7 mins, and the DNA product was purified (20 ul) with a Zymo column (Zymo Research, #D4013). Tagmented DNA was used for the 1st PCR amplification (initial denaturation at 98° C. for 2 min; 25 cycles of denaturation at 98° C. for 10 s, annealing at 58° C. for 10 s, and elongation at 72° C. for 20 s. The final cycle was followed by extension at 72° C. for 5 min) using Platinum™ SuperFi DNA polymerase (Thermo) with i5 primer and one or more tag primers. See, Table 1.

Depending on the type of PE-tag being employed. For in vitro PE-tag, one Illumina library per sample was prepared from tagmented gDNA with the i5+tag primer_R primer in the 1^{st} PCR amplification. For PE-tag in cells or in vivo PE-tag, two different Illumina libraries were prepared from tagmented gDNA using either the i5+Tag primer_F or i5+Tag primer_R primer in the 1^{st} PCR amplification. The i7 index was added in the 2nd PCR amplification (initial denaturation at 98° C. for 2 min; 30 cycles of denaturation at 98° C. for 10 s, annealing at 65° C. for 10 s, and elongation at 72° C. for 30 s. The final cycle was followed by extension at 72° C. for 5 min) using Platinum™ SuperFi DNA polymerase (Thermo) with i5 primer and i7 primers. See, Table 1. The PCR product was cleaned up with Ampure XP SPRI beads (Agencourt, 0.9× reaction volume). Completed libraries were quantified by Tapestation and Qubit (Agilent), pooled with at equal molar ratio and sequenced with 150 bp paired-end reads on an Illumina MiniSeq instrument.

### Example VII

### PE-tag Data Analysis

The PE-tag analysis pipeline was built using python code. Code is available at rdrr.io/github/LihuaJulieZhu/GUIDEseq/man/PEtagAnalysis.html. Briefly, it consists of the following steps:
i. Demultiplexing and UMI extraction. Raw BCL files were converted and demultiplexed using the appropriate i5 and i7 sequencing barcodes, allowing up to one mismatch in each barcode. Unique molecular identifiers (UMIs) for each read were extracted for further downstream analysis.
ii. Raw reads were processed with fastqc (Version 0.11.9) and trim galore (Version 0.6.5) (https://www.bioinformatics.babraham.ac.uk/projects/) to remove reads with low quality and trim adapters.
iii. Create a reference sequence based on the donor map. Build index files for the reference using bowtie2-index, version 2.4.0.
iv. Alignment analysis. Paired reads were then globally aligned (end-to-end mode) to the donor genome (mm 10/hg38) and all the reference amplicons using bowtie2's very sensitive parameter. Finally, Samtools (version 0.1.19) was used to create an index-sorted bam file. For off-targeting sites, we used these parameters: the maximum mismatch is 6 base pairs, DNA/RNA bulge is 1 base pair and NNG/NGN for PAM filtering.

### Example VII

### PE-tag Open-Source Analysis Software

To enable the broad use of PE-tag for genome-wide detection of prime editor off-target sites, we developed a freely available, open-source R package for the analysis of PE-tag deep sequencing data. The package performs full end-to-end analysis of PE-tag sequencing data with a single command and returns a data frame, containing all input peaks with potential gRNA binding sites, mismatch number and positions, alignment to the input gRNA, predicted cleavage score, PBS (primer binding sequence), and HA (homology arm sequence). Source code and running instructions are freely available online. See, rdrr.io/github/LihuaJulieZhu/GUIDEseq/man/PEtagAnalysis.html.

### Example IX

### Targeted Amplicon Deep Sequencing

Genomic DNA was isolated for prime editing analysis from prime editor treated cells or the frozen liver of mice injected with pegRNA+PE2. Genomic loci spanning the target and off-target sites were PCR amplified with locus-specific primers carrying tails complementary to the Truseq adapters.

For all pegRNAs, gene editing was validated for the top 5 to 10 potential off-target sites identified by PE-tag. Targeted amplicon deep sequencing libraries were prepared by two PCR steps. 200 ng of genomic DNA was used for the 1^{st} PCR using Phusion master mix (Thermo) with locus specific primers that contain tails. PCR products from the 1^{st} PCR were used for the 2^{nd} PCR with i5 primers and i7 primers to complete the adaptors and include the i5 and i7 indices. See, Table 3.

TABLE 3
Sequences of primers used for pegRNA cloning

| pegRNA | Sequence (5′-3′) |
|---|---|
| HEK4_F | GTGGAAAGGACGAAACACCGGCACTGCGGCTGGAGGTGGgttttagagctagaaatag |
| HEK4 7-7 | CGGTATCGATAAGCTTGATATCGAATTCAAAAAAGGCTGGAGGACGACTCTGGGGGTGGACCGACTCGGTCCCAC |
| HEK4 7-16 | GGTATCGATAAGCTTGATATCGAATTCAAAAAAGGCTGGAGGACGACTCTGGGGGTTAAAGCGGAGGACCGACTCGG |
| | TCCCAC |
| HEK4 7-20 | GGTATCGATAAGCTTGATATCGAATTCAAAAAAGGCTGGAGGACGACTCTGGGGGTTAAAGCGGAGACTGGACCGAC |
| | TCGGTCCCAC |
| HEK4 11-7 | CGGTATCGATAAGCTTGATATCGAATTCAAAAAAGGCTGGAGGTGTCACGACTCTGGGGGTGGACCGACTCGGTCCC |
| | AC |
| HEK4 11-16 | GGTATCGATAAGCTTGATATCGAATTCAAAAAAGGCTGGAGGTGTCACGACTCTGGGGGTTAAAGCGGAGGACCGAC |
| | TCGGTCCCAC |
| HEK4 11-20 | GGTATCGATAAGCTTGATATCGAATTCAAAAAAGGCTGGAGGTGTCACGACTCTGGGGGTTAAAGCGGAGACTGGAC |
| | CGACTCGGTCCCAC |
| HEK4 20-7 | TCGATAAGCTTGATATCGAATTCAAAAAAGGCTGGAGGGTTGTCCGCTGTCACGACTCTGGGGGTGGACCGACTCGG |
| | TCCCAC |
| HEK4 20-16 | ATCGATAAGCTTGATATCGAATTCAAAAAAGGCTGGAGGGTTGTCCGCTGTCACGACTCTGGGGGTTAAAGCGGAGG |
| | ACCGACTCGGTCCCAC |
| HEK4 20-20 | ATCGATAAGCTTGATATCGAATTCAAAAAAGGCTGGAGGGTTGTCCGCTGTCACGACTCTGGGGGTTAAAGCGGAGA |
| | CTGGACCGACTCGGTCCCAC |
| VEGFA 20-8_F | TGGAAAGGACGAAACACCGACCCCCTCCACCCCGCCTCGTTTTAGAGCTAGAAATAG |
| VEGFA 20-8_R | TCGATAAGCTTGATATCGAATTCAAAAAATCCACCCCGCGTTGTCCGCTGTCACGACTCCTCCGGGCGGACCGACTC |
| | GGTCCCACT |
| PCSK9 20-8_F | GTGGAAAGGACGAAACACCGAGCAGCAGCGGCGGCAACAGGTTTTAGAGCTAGAAATAGC |
| PCSK9 20-8_R | TCGATAAGCTTGATATCGAATTCAAAAAAGCGGCGGCAAGTTGTCCGCTGTCACGACTCCAGCGGCAGGACCGACTC |
| | GGTCCCACT |
| HEK4 G-T-7 | TCGATAAGCTTGATATCGAATTCAAAAAATCCACCCCGCCGCCGGGCGCGGACCGACTCGGTCCCAC |
| HEK4 G-T-16 | TCGATAAGCTTGATATCGAATTCAAAAAATCCACCCCGCCGCCGGGCGCGGGCTCCGGACCGACTCGGTCCCAC |
| HEK4 G-T-20 | TCGATAAGCTTGATATCGAATTCAAAAAATCCACCCCGCCGCCGGGCGCGGGCTCCGGCCCGGACCGACTCGGTCCC |
| | AC |
| HEK4 1-Del-7 | TCGATAAGCTTGATATCGAATTCAAAAAATCCACCCCGCCCCGGGCGCGGACCGACTCGGTCCCACT |
| HEK4 1-Del-16 | ATCGATAAGCTTGATATCGAATTCAAAAAATCCACCCCGCCCCGGGCGCGGGCTCCGGACCGACTCGGTCCCACT |
| HEK4 1-Del-20 | ATCGATAAGCTTGATATCGAATTCAAAAAATCCACCCCGCCCCGGGCGCGGGCTCCGGCCCGGACCGACTCGGTCCC |
| | ACT |
| HEK4 1-Inser-7 | TCGATAAGCTTGATATCGAATTCAAAAAATCCACCCCGCCGTCCGGGCGGACCGACTCGGTCCCACT |
| HEK4 1-Inser-16 | ATCGATAAGCTTGATATCGAATTCAAAAAATCCACCCCGCCGTCCGGGCGCGGGCTCCGGACCGACTCGGTCCCAC |
| HEK4 1-Inser-20 | ATCGATAAGCTTGATATCGAATTCAAAAAATCCACCCCGCCGTCCGGGCGCGGGCTCCGGCCGGACCGACTCGGTCC |
| | CACTT |
| CDH4 20-10_F | GTGGAAAGGACGAAACACCGTGCACTGCGGCCGGAGGAGGGTTTTAGAGCTAGAAATAG |
| CDH4 20-10_R | TCGATAAGCTTGATATCGAATTCAAAAAATGCGGCCGGAGGGTTGTCCGCTGTCACGACTCAGGTGGAGGAGGACCG |
| | ACTCGGTCCCAC |

PCR products were purified with Ampure beads (0.9× reaction volume) and eluted with 25 ul of TE buffer, and were quantified by Tapestation and Qubit. Equal mole of each amplicon was pooled and sequenced using Illumina Miniseq. Amplicon sequencing data was analyzed with CRISPResso. Clement, K. et al. Nat Biotechnol 37, 224-226 (2019).

### Example X

### Statistical Analysis

Statistical analyses for plotted data were performed using GraphPad Prism 8.4. Sample size was not pre-determined by statistical methods, but rather, based on preliminary data. Group allocation was performed randomly. In all studies, data represent biological replicates (n) and are depicted as mean±s.d. as indicated in the figure legends. Comparison of mean values was conducted with unpaired, two-tailed Student's t-test; one-way ANOVA; or two-way ANOVA with Tukey's multiple comparisons test, as indicated in the figure legends. In all analyses, P values <0.05 were considered statistically significant. P values were adjusted using the Benjamini & Hochberg (BH) method to correct for multiple inferences in each experiment.

### Example XI

### Data Availability

Illumina Sequencing data have been submitted to the Sequence Read Archive. These datasets are available under BioProject Accession number PRJNA811252. See, ncbi.nlm.nih.gov/bioproject/PRJNA811252. The authors declare that all other data supporting the findings of this study are available within the paper and its Supplementary Information files or upon reasonable request. Backbone plasmids used for pegRNA and sgRNA cloning are available from Addgene.

### Example XII

### Code Availability

The software used for data analysis is available at Github. See, rdrr.io/github/-LihuaJulieZhu/GUIDEseq/man/PEtagAnalysis.html.

1. A method, comprising:
a) providing:
i) an oligonucleotide sequence or a genomic sequence comprising a target sequence;
ii) a composition comprising a Cas9 nickase (nCas9) protein and a reverse transcriptase protein;
iii) a prime editor guide ribonucleic acid (pegRNA) comprising a spacer sequence that is at least partially complementary to said target sequence and an amplification tag element;
iv) a Tn5 transposase enzyme; and
v) a universal adaptor;
b) hybridizing said spacer sequence to said target sequence;
c) nicking said target sequence with said nCas9 protein to create a free 3′ DNA end;
d) incorporating said amplification tag element into said free 3′ DNA end with said reverse transcriptase;
e) incorporating said universal adaptor into said oligonucleotide sequence or said genomic sequence with said Tn5 transposase enzyme;
f) amplifying said oligonucleotide sequence or said genomic sequence between said amplification tag element and said universal adaptor to create a plurality of amplicons; and
g) sequencing said plurality of amplicons to identify at least one of the following:
i) an on-target prime editor recognition sequence when said spacer sequence is fully-cognate with said target sequence;
ii) an off-target prime editor recognition sequence when said spacer sequence is near-cognate with said target sequence; or
iii) a prime editor editing efficiency at the on-target or off-target prime editor recognition sites.

2-7. (canceled)

8. The method of claim 1, wherein said amplifying further comprises a first amplification primer complementary to said amplification tag element.

9-10. (canceled)

11. The method of claim 1, wherein said sequencing further comprises a second amplification primer complementary to said univeral adaptor.

12-13. (canceled)

14. The method of claim 1, wherein said universal adaptor comprises at least one unique molecular identifier (UMI) sequence.

15-17. (canceled)

18. The method of claim 1, wherein said target sequence is associated with a pathological medical condition.

19. A composition comprising a Cas9 nickase (nCas9) protein and a prime editor guide ribonucleic acid (pegRNA), said pegRNA encoding an amplification tag element.

20. The composition of claim 19, wherein said pegRNA further comprises a spacer sequence that is at least partially complementary to a prime editor recognition sequence.

21. The composition of claim 19, wherein said composition further comprises a reverse transcriptase protein.

22. The composition of claim 21, wherein said nCas9 protein and said reverse transcriptase protein are a fusion protein.

23. The composition of claim 19, wherein said pegRNA further comprises a reverse transcriptase template sequence.

24-33. (canceled)

26. The composition of claim 19, further comprising a kit, comprising:
a) a first container comprising said Cas9 nickase (nCas9) protein and said reverse transcriptase;
b) a second container comprising said prime editor guide ribonucleic acid (pegRNA);
c) a third container comprising a control pegRNA;
d) a fourth container comprising a first primer set configured to hybridize with a control genomic region; and
e) a fifth container comprising a second primer set configured to hybridize with said amplification tag element.

27-29. (canceled)

30. The composition of claim 26, wherein said kit further comprises instructional materials for selected from the group consisting of the use of reagents in the performance of PE-tag, the generation of sequencing libraries, the design parameters to construct a custom pegRNA, the design parameters to construct locus-specific primers and the use of PE-tag sequence analysis software.

31-33. (canceled)

34. A method, comprising:
a) providing:
i) a biological sample comprising an oligonucleotide sequence or a genomic sequence encoding a mutated target sequence derived from a patient exhibiting at least one symptom of a pathological medical condition;
ii) a composition comprising a Cas9 nickase (nCas9) protein and a reverse transcriptase protein;
iii) a pegRNA comprising a spacer sequence that is at least partially complementary to said mutated target sequence and an amplification tag element;
iv) a Tn5 transposase enzyme; and
v) a universal adaptor;
b) hybridizing said pegRNA spacer sequence to said mutated target sequence;
c) nicking said oligonucleotide sequence or genomic sequence with said nCas9 protein to create a free 3′ DNA end;
d) incorporating said amplification tag element into said free 3′ DNA end with said reverse transcriptase;
e) appending the universal adaptor to said oligonucleotide sequence or said genomic sequence with said Tn5 transposase enzyme;
f) amplifying said oligonucleotide sequence or said genomic sequence between said amplification tag element and said universal adaptor to create a plurality of amplicons; and
g) sequencing said plurality of amplicons to identify at least one of the following:
i) an on-target gene editing site when said spacer sequence is fully cognate with said mutated target sequence;
ii) an off-target gene editing site when said spacer sequence is near-cognate with said mutated target sequence; or
iii) a gene editing efficiency at said on-target site or said off-target site.

35-36. (canceled)

37. The method of claim 346, wherein said pathological medical condition is a genetic disease or disorder.

38. The method of claim 34, wherein said method further comprises in vitro editing of said on-target gene editing site or said off-target gene editing site with a prime editor protein complex comprising a pegRNA with a reverse transcriptase template encoding a wild type target site to identify a validated on-target site or a validated off-target site.

39-41. (canceled)

42. The method of claim 34, wherein said nCas9 nickase protein and reverse transcriptase protein are a fusion protein.

43. (canceled)

44. The method of claim 34, wherein said amplifying further comprises a first amplification primer complementary to said amplification tag element.

45-46. (canceled)

47. The method of claim 34, wherein said sequencing further comprises a second amplification primer complementary to said universal adaptor.

48-49. (canceled)

50. The method of claim 34, wherein said universal adaptor comprises at least one unique molecular identifier (UMI) sequence.

51-53. (canceled)

54. The method of claim 34, wherein said pathological medical condition is a genetic disease or disorder.
